(19) United States
(12) Patent Application Publication
(10) Pub. No.: US 2016/0158329 A1
(43) Pub. Date: Jun. 9, 2016

(54) VACCINES COMPRISING LEISHMANIA POLYPEPTIDES FOR THE TREATMENT AND DIAGNOSIS OF LEISHMANIASIS

(71) Applicant: INFECTIOUS DISEASE RESEARCH INSTITUTE, Seattle, WA (US)

(72) Inventors: Jeff GUDERIAN, Seattle, WA (US); Malcolm DUTHIE, Seattle, WA (US); Steven G. REED, Bellevue, WA (US)

(73) Assignee: Infectious Disease Research Institute, Seattle, WA (US)

(21) Appl. No.: 14/780,494

(22) PCT Filed: Mar. 28, 2014
(86) PCT No.: PCT/US2014/032273

§ 371 Date: Sep. 25, 2015

Related U.S. Application Data
(60) Provisional application No. 61/806,370, filed on Mar. 28, 2013.
(60) Provisional application No. 61/822,530, filed on May 13, 2013.

(51) Int. Cl. A61K 39/008; G01N 33/569

(52) U.S. Cl. CPC A61K 39/008; G01N 33/56905; G01N 2469/20

(57) ABSTRACT

Compositions and methods for preventing, treating and detecting leishmaniasis are disclosed. The compositions generally comprise polypeptides comprising one or more Leishmania antigens as well as polynucleotides encoding such polypeptides.

### CROSS-REFERENCE TO RELATED PATENT APPLICATIONS

This application claims the priority benefit of U.S. provisional application Ser. Nos. 61/806,370, filed Mar. 28, 2013, and 61/822,530, filed May 13, 2013, all of which are incorporated herein by reference in their entirety.

### BACKGROUND

1. Technical Field

The present invention relates generally to compositions and methods for preventing, treating and detecting leishmaniasis in patients. More particularly, the invention relates to compositions and methods comprising Leishmania antigens and fusion polypeptides, as well as polynucleotides encoding such antigens and fusion polypeptides.

2. Description of the Related Art

Leishmania organisms are obligate intracellular parasites that cause a large clinical spectrum of diseases named leishmaniasis. Leishmania organisms are intracellular protozoan parasites of the genus Leishmania. Leishmania organisms target host macrophages; thus causing a wide spectrum of clinical diseases in humans and domestic animals, primarily dogs. In some infections, the parasite may lie dormant for many years. In other cases, the host may develop one of a variety of forms of leishmaniasis. Leishmaniases are roughly classified into three types of diseases, cutaneous leishmaniasis (CL), mucosal leishmaniasis (ML) and visceral leishmaniasis (VL), according to the clinical manifestations.

Leishmaniasis is a serious problem in much of the world, including Brazil, China, East Africa, India and areas of the Middle East. The disease is also endemic in the Mediterranean region, including southern France, Italy, Greece, Spain, Portugal and North Africa. The number of cases of leishmaniasis has increased dramatically in the last 20 years, and millions of cases of this disease now exist worldwide. About 2 million new cases are diagnosed each year, 25% of which are visceral leishmaniasis.

Visceral leishmaniasis (VL) has been reported in 88 countries, but roughly 90% of VL cases occur in Brazil, India, Sudan, Bangladesh, and Nepal (Mendez et al. J Immunol 2001; 166(8): pp. 5122-8). The annual incidence is estimated to be approximately 500,000 cases of VL, and the population at risk is 350 million (Engwerda et al. Eur J Immunol 1998; 28(2): pp. 669-80; Squires et al. J Immunol 1989; 143(12): pp. 4244-9). Visceral leishmaniasis, generally caused by species of the L. donovani complex, i.e. L. donovani and L. infantum (chagasi). L. donovani is the causative agent of visceral leishmaniasis in Africa and Asia, L. infantum/chagasi in Mediterranean countries and in the New World (Piedrafita et al. J Immunol 1999; 163(3): pp. 1467-72). VL is a severe debilitating disease that evolves with visceral infection involving the spleen, liver and lymph nodes, which, untreated, is generally a fatal disease. Symptoms of acute visceral leishmaniasis include hepatosplenomegaly, fever, leukopenia, anemia and hypergammaglobulinemia. Active VL is generally fatal unless properly treated.

Leishmania parasites are transmitted by the bite of sandflies and the infecting promastigotes differentiate into and replicate as amastigotes within macrophages in the mammalian host. In common with other intracellular pathogens, cellular immune responses are critical for protection against leishmaniasis. Th1 immune responses play an important role in mediating protection against Leishmania, including roles for CD4+ and CD8+ T cells, IFN-γ, IL-12, TNF-a and NO, whereas inhibitory effects have been reported for IL-10 and TGF-B (Engwerda et al. Eur J Immunol 1998; 28(2): pp. 669-80; Murphy et al. Eur J Immunol. 2001; 31(10): pp. 2848-56; Murray et al. J Exp Med. 1999; 189(4): pp. 741-6; Murray et al. Infect Immun. 2000; 68(11): pp. 6289-93; Squires et al. J Immunol 1989; 143(12): pp. 4244-96; Taylor and Murray. J Exp Med. 1997; 185(7): pp. 1231-9; Kaye and Bancroft. Infect Immun. 1992; 60(10): pp. 4335-42; Stern et al. J Immunol. 1988; 140(11): pp. 3971-7; Wilson et al. J Immunol. 1998; 161(11): pp. 6148-55).

Immunization against leishmaniasis in animal models can be effected by delivery of antigen-encoding DNA vectors (Gurunathan et al. J Exp Med. 1997; 186(7): pp. 1137-47; Piedrafita et al. J Immunol. 1999; 163(3):1467-72; Mendez et al. J Immunol. 2001; 166(8): pp. 5122-8) or by administration of proteins formulated with Th1-inducing adjuvants including IL-12 (Afonso et al. Science. 1994; 263(5144): pp. 235-7; Stobie et al. Proc Natl Acad Sci USA. 2000; 97(15): pp. 8427-32; Kenney et al. J Immunol. 1999; 163(8): pp. 4481-8) or TLR ligands such as CpG oligonucleotides (Rhee et al. J Exp Med. 2002; 195(12): pp. 1565 [symbol] 73; Stacey and Blackwell. Infect Immun. 1999; 67(8): pp. 3719-26; Walker et al. Proc Nat/Acad Sci USA. 1999; 96(12): pp. 6970-5) and monophosphoryl lipid A (Coler et al. Infect Immun. 2002; 70(8): pp. 4215-25; Skeiky et al. Vaccine. 2002; 20(2728): pp. 3292-303).

In spite of some evidence that sub-unit vaccines may be effective in certain models of VL (Basu et al. J Immunol. 2005; 174(11): pp. 7160-71; Stager et al. J Immunol. 2000; 165(12): pp. 7064-71; Ghosh et al. Vaccine. 2001; 20(12): pp. 59-66; Wilson et al. Infect lmmun. 1995; 63(5): pp. 2062-9; Tewary et al. J Infect Dis. 2005; 191(12): pp. 2130-7; Aguilar-Be et al. Infect lmmun. 2005; 73(2): pp. 812-9. Rafati et al. Vaccine. 2006; 24(12):2169-75), progress toward defining antigen candidates effective against VL in vivo has been lacking.

Strategies employing vaccines consisting of whole organisms for preventing or treating leishmaniasis have not been effective in humans. In addition, more effective reagents are needed for accurately diagnosing leishmaniasis in patients. Accordingly, there remains a significant need for immunogenic compositions and vaccines that can effectively prevent, treat and/or diagnose leishmaniasis in humans and other mammals (e.g., canines). The present invention fulfills these needs and offers other related advantages

### BRIEF SUMMARY

The present invention provides compositions, kits and methods for preventing, treating and detecting leishmaniasis.

In one aspect, the invention provides an immunogenic portion of a Leishmania mitochondrial HSP 70 (mtHSP70) polypeptide, wherein the immunogenic portion comprises a carboxy terminal region sequence of the mtHSP70.

In another aspect, the invention provides a polypeptide comprising an immunogenic portion of a Leishmania mtHSP70 polypeptide, wherein the immunogenic portion comprises a carboxy terminal region sequence of the mtHSP70.

In some embodiments, the mtHSP70 polypeptide is a L. infantum, a L. donovani, a L. major, a L. mexicana, or a L. braziliensis mtHSP70 polypeptide. In some embodiments, the carboxy terminal region of the mtHSP70 comprises the amino acid sequence of SEQ ID NO: 21, 22, 23, or 24, or a sequence having at least 90% identity to SEQ ID NO: 21, 22, 23, or 24.

In some embodiments, the polypeptide is a fusion polypeptide. In some embodiments, the polypeptide further comprises a Leishmania putative carboxypeptidase (CxP) polypeptide. In some embodiments, the CxP polypeptide is a L. infantum, a L. donovani, L. major, L. mexicana, or L. braziliensis CxP polypeptide. In some embodiments, the CxP polypeptide comprises the amino acid sequence of SEQ ID NO: 25, 26, 27, 28, or 29, or a sequence having at least 90% identity to SEQ ID NO: 25, 26, 27, 28, or 29.

In some embodiments, the fusion polypeptide further comprises a Leishmania cysteine protease gene B (CpB) polypeptide, a Leishmania histone of H2BN (H2BN) polypeptide, a Leishmania A2 polypeptide, a p21 antigen (p21) polypeptide, a Leishmania thiol specific antioxidant (TSA) polypeptide, a Leishmania putative eukaryotic initiation factor4a (Leif) polypeptide, a Leishmania CxP polypeptide, a Malate Dehydrogenase polypeptide (MDH) and/or a Leishmania Alph Tubulin polypeptide (aT). In some embodiments, the fusion polypeptide further comprises one or more of the following polypeptides: a Leishmania cysteine protease gene B (CpB) polypeptide, a Leishmania histone of H2BN (H2BN) polypeptide, a Leishmania A2 polypeptide, a p21 antigen (p21) polypeptide, a Leishmania thiol specific antioxidant (TSA) polypeptide, a Leishmania putative eukaryotic initiation factor4a (Leif) polypeptide, a Leishmania CxP polypeptide, a Malate Dehydrogenase polypeptide (MDH) and a Leishmania Alph Tubulin polypeptide (aT). In some embodiments, the fusion polypeptide comprises a Leishmania mtHSP70 polypeptide, a Leishmania CxP polypeptide, and a Leishmania p21 polypeptide. In some embodiments, the fusion polypeptide comprises a Leishmania mtHSP70 polypeptide, a Leishmania CxP polypeptide, a Leishmania H2BN polypeptide, and a Leishmania p21 polypeptide. In some embodiments, the fusion polypeptide comprises a Leishmania mtHSP70 polypeptide, a Leishmania CxP polypeptide, a Leishmania A2 polypeptide, and a Leishmania p21 polypeptide. In some embodiments, the fusion polypeptide comprises a Leishmania mtHSP70 polypeptide, a Leishmania A2 polypeptide, and a Leishmania p21 polypeptide. In some embodiments, the fusion polypeptide comprises a Leishmania mtHSP70 polypeptide, a Leishmania A2 polypeptide, a Leishmania p21 polypeptide, and a Leishmania NH polypeptide. In some embodiments, the fusion polypeptide comprises a Leishmania mtHSP70 polypeptide, a Leishmania CxP polypeptide, a Leishmania H2BN polypeptide, and a Leishmania A2 polypeptide. In some embodiments, the fusion polypeptide comprises a Leishmania mtHSP70 polypeptide, a Leishmania H2BN polypeptide, and a Leishmania A2 polypeptide. In some embodiments, the fusion polypeptide comprises a Leishmania mtHSP70 polypeptide, a Leishmania H2BN polypeptide, a Leishmania A2 polypeptide, and a Leishmania NH polypeptide. In some embodiments, the fusion polypeptide comprises a Leishmania mtHSP70 polypeptide, a Leishmania CpB polypeptide, a Leishmania H2BN polypeptide, and a Leishmania A2 polypeptide. In some embodiments, the fusion polypeptide comprises a Leishmania mtHSP70 polypeptide, a Leishmania CpB polypeptide, and a Leishmania A2 polypeptide. In some embodiments, the fusion polypeptide comprises a Leishmania mtHSP70 polypeptide, a Leishmania CpB polypeptide, a Leishmania A2 polypeptide, and a Leishmania NH polypeptide. In some embodiments, the fusion polypeptide comprises a Leishmania mtHSP70 polypeptide, a Leishmania NH polypeptide, and a Leishmania CpB polypeptide. In some embodiments, the fusion polypeptide comprises a Leishmania mtHSP70 polypeptide, a Leishmania NH polypeptide, a Leishmania CpB polypeptide, and a Leishmania H2BN polypeptide. In some embodiments, the fusion polypeptide comprises a Leishmania mtHSP70 polypeptide, a Leishmania MDH polypeptide, a Leishmania CpB polypeptide, and a Leishmania H2BN polypeptide. In some embodiments, the fusion polypeptide comprises a Leishmania mtHSP70 polypeptide, a Leishmania MDH polypeptide, a Leishmania aT polypeptide, and a Leishmania H2BN polypeptide. In some embodiments, the fusion polypeptide comprises a Leishmania mtHSP70 polypeptide, a Leishmania aT polypeptide, a Leishmania CpB polypeptide, and a Leishmania H2BN polypeptide.

In some embodiments, the CpB polypeptide is a L. infantum, a L. donovani, L. major, L. Mexicana, or L. braziliensis CpB polypeptide. In some embodiments, the CpB polypeptide comprises the amino acid sequence of SEQ ID NO: 30 or a sequence having at least 90% (e.g., 91%, 92%, 93%, 94%, 95%, 96%, 97%, 98%, 99%) identity to SEQ ID NO: 30.

In some embodiments, the H2BN polypeptide is a L. infantum, a L. donovani, L. major, L. mexicana, or L. braziliensis H2BN polypeptide. In some embodiments, the H2BN comprises the amino acid sequence of SEQ ID NO: 31 or a sequence having at least 90% (e.g., 91%, 92%, 93%, 94%, 95%, 96%, 97%, 98%, 99%) identity to SEQ ID NO: 31.

In some embodiments, the A2 polypeptide is a L. infantum, a L. donovani, L. major, L. mexicana, or L. braziliensis A2 polypeptide. In some embodiments, the A2 polypeptide comprises the amino acid sequence of SEQ ID NO: 32 or 37, or a sequence having at least 90% (e.g., 91%, 92%, 93%, 94%, 95%, 96%, 97%, 98%, 99%) identity to SEQ ID NO: 32 or 37.

In some embodiments, the p21 polypeptide is a L. infantum, a L. donovani, L. major, L. mexicana, or L. braziliensis p21 polypeptide. In some embodiments, the p21 polypeptide comprises the amino acid sequence of SEQ ID NO: 33, or a sequence having at least 90% (e.g., 91%, 92%, 93%, 94%, 95%, 96%, 97%, 98%, 99%) identity to SEQ ID NO: 33.

In some embodiments, the NH polypeptide is a L. infantum, a L. donovani, L. major, L. mexicana, or L. braziliensis NH polypeptide. In some embodiments, the NH polypeptide comprises the amino acid sequence of SEQ ID NO: 36, or a sequence having at least 90% (e.g., 91%, 92%, 93%, 94%, 95%, 96%, 97%, 98%, 99%) identity to SEQ ID NO: 36.

In some embodiments, the CxP polypeptide is a L. infantum, a L. donovani, L. major, L. mexicana, or L. braziliensis CxP. In some embodiments, the CxP polypeptide comprises an immunogenic portion of a CxP polypeptide. In some embodiments, the CxP polypeptide comprises the amino acid sequence of SEQ ID NO: 25, 26, 27, 28, or 29 or a sequence having at least 90% (e.g., 91%, 92%, 93%, 94%, 95%, 96%, 97%, 98%, 99%) identity to SEQ ID NO: 25, 26, 27, 28, or 29.

In some embodiments, the aT polypeptide is a L. infantum, a L. donovani, L. major, L. mexicana, or L. braziliensis aT polypeptide. In some embodiments, the NH polypeptide comprises the amino acid sequence of SEQ ID NO: 38, or a sequence having at least 90% (e.g., 91%, 92%, 93%, 94%, 95%, 96%, 97%, 98%, 99%) identity to SEQ ID NO: 38.

In some embodiments, the MDH polypeptide is a L. infantum, a L. donovani, L. major, L. mexicana, or L. braziliensis MDH polypeptide. In some embodiments, the MDH polypeptide comprises the amino acid sequence of SEQ ID NO: 39, or a sequence having at least 90% (e.g., 91%, 92%, 93%, 94%, 95%, 96%, 97%, 98%, 99%) identity to SEQ ID NO: 39.

In some embodiments, the fusion polypeptide comprises sequences from at least two, at least three, or at least four different Leishmania strains.

In some embodiments, the fusion polypeptide comprises the amino acid sequence of SEQ ID NO: 2, 4, 6, 8, 14, 16, 18, 20, 41, 43, 45, 47, or 49 or an amino acid sequence having at least 90% (e.g., 91%, 92%, 93%, 94%, 95%, 96%, 97%, 98%, 99%) identity to SEQ ID NO: 2, 4, 6, 8, 14, 16, 18, 20, 41, 43, 45, 47, or 49.

In another aspect, the invention provides a fusion polypeptide comprising a Leishmania CxP polypeptide and a second Leishmania polypeptide.

In some embodiments, the CxP polypeptide is a L. infantum, a L. donovani, L. major, L. mexicana, or L. braziliensis CxP. In some embodiments, the CxP polypeptide comprises an immunogenic portion of a CxP polypeptide. In some embodiments, the CxP polypeptide comprises the amino acid sequence of SEQ ID NO: 25, 26, 27, 28, or 29 or a sequence having at least 90% (e.g., 91%, 92%, 93%, 94%, 95%, 96%, 97%, 98%, 99%) identity to SEQ ID NO: 25, 26, 27, 28, or 29.

In some embodiments, the second polypeptide comprises a Leishmania H2BN polypeptide and a NH polypeptide. In some embodiments, the H2BN polypeptide is a L. infantum, a L. donovani, L. major, L. mexicana, or L. braziliensis H2BN polypeptide. In some embodiments, the H2BN polypeptide comprises the amino acid sequence of SEQ ID NO: 31 or a sequence having at least 90% (e.g., 91%, 92%, 93%, 94%, 95%, 96%, 97%, 98%, 99%) identity to SEQ ID NO: 31. In some embodiments, the NH polypeptide is a L. infantum, a L. donovani, L. major, L. mexicana, or L. braziliensis NH polypeptide. In some embodiments, the NH polypeptide comprises the amino acid sequence of SEQ ID NO: 36 or a sequence having at least 90% (e.g., 91%, 92%, 93%, 94%, 95%, 96%, 97%, 98%, 99%) identity to SEQ ID NO: 36. In some embodiments, the fusion polypeptide comprises the amino acid sequence of SEQ ID NO: 10 or a sequence having at least 90% (e.g., 91%, 92%, 93%, 94%, 95%, 96%, 97%, 98%, 99%) identity to SEQ ID NO: 10.

In some embodiments, the second polypeptide comprises a Leishmania TSA polypeptide and a Leishmania Leif polypeptide. In some embodiments, the TSA polypeptide is a L. infantum, a L. donovani, L. major, L. mexicana, or L. braziliensis TSA polypeptide. In some embodiments, the TSA polypeptide comprises the amino acid sequence of SEQ ID NO: 34 or a sequence having at least 90% (e.g., 91%, 92%, 93%, 94%, 95%, 96%, 97%, 98%, 99%) identity to SEQ ID NO: 34. In some embodiments, the Leif polypeptide is a L. infantum, a L. donovani, L. major, L. mexicana, or L. braziliensis Leif polypeptide. In some embodiments, the Leif polypeptide comprises the amino acid sequence of SEQ ID NO: 35 or a sequence having at least 90% (e.g., 91%, 92%, 93%, 94%, 95%, 96%, 97%, 98%, 99%) identity to SEQ ID NO: 35. In some embodiments, the fusion polypeptide comprises the amino acid sequence of SEQ ID NO: 12 or a sequence having at least 90% (e.g., 91%, 92%, 93%, 94%, 95%, 96%, 97%, 98%, 99%) identity to SEQ ID NO: 12.

In another aspect, the invention provides an isolated polynucleotide encoding a polypeptide (including fusion polypeptides) as described herein, for example, the fusion polypeptide comprises the amino acid sequence of SEQ ID NO: 2, 4, 6, 8, 10, 12, 14, 16, 18, 20, 41, 43, 45, 47, or 49 or an amino acid sequence having at least 90% (e.g., 91%, 92%, 93%, 94%, 95%, 96%, 97%, 98%, 99%) identity to SEQ ID NO: 2, 4, 6, 8, 10, 12, 14, 16, 18, 20, 41, 43, 45, 47, or 49. In some embodiments, the polynucleotide comprises a sequence selected from the group consisting of SEQ ID NOS:1, 3, 5, 7, 9, 11, 13, 15, 17, 19, 40, 42, 44, 46 and 48, or a sequence having at least 90% (e.g., 91%, 92%, 93%, 94%, 95%, 96%, 97%, 98%, 99%) identity to a sequence selected from the group consisting of SEQ ID NOS:1, 3, 5, 7, 9, 11, 13, 15, 17, 19, 40, 42, 44, 46 and 48.

In another aspect, the invention provides a composition comprising a polypeptide (including fusion polypeptides) as described herein and/or a polynucleotide encoding a polypeptide as described herein, in combination with at least one immunostimulant. Many immunostimulants are known and can be used in the compositions herein, illustrative examples of which include, but are not limited to, a CpG-containing oligonucleotide, synthetic lipid A, MPLTM, 3D-MPLTM, saponins, saponin mimetics, AGPs, Toll-like receptor agonists, or a combination thereof. Other illustrative immunostimulants comprise, for example, aTLR4 agonist, a TLR7/8 agonist and/or a TLR9 agonist. Still other immunostimulants comprise, for example, imiquimod, gardiquimod and/or resiquimod.

In another aspect, the invention provides a method for stimulating an immune response against Leishmania in a mammal comprising administering to a mammal in need thereof a composition as described herein.

In another aspect, the invention provides a method for detecting Leishmania infection in a biological sample, comprising: (a) contacting a biological sample with a polypeptide (including a fusion polypeptide) as described herein; and (b) detecting in the biological sample the presence of antibodies that bind to the fusion polypeptide, thereby detecting Leishmania infection in a biological sample. Any suitable biological sample type may be analyzed by the method, illustrative examples of which may include, for example, sera, blood and saliva.

In certain embodiments of the disclosed diagnostic methods, the polypeptide (including a fusion polypeptide) is bound to a solid support. Accordingly, the present invention further provides diagnostic reagents comprising a polypeptide (including a fusion polypeptide) as described herein, immobilized on a solid support.

Diagnostic kits for detecting Leishmania infection in a biological sample are also provided, generally comprising a polypeptide (including a fusion polypeptide) as described herein and a detection reagent. It will be understood that the kit may employ a polypeptide (including a fusion polypeptide) of the invention in any of a variety of assay formats known in the art, including, for example, a lateral flow test strip assay, a dual path platform (DPP) assay and an ELISA assay. These kits and compositions of the invention can offer valuable point of care diagnostic information. Furthermore, the kits and compositions can also be advantageously used as test-of-cure kits for monitoring the status of infection in an infected individual over time and/or in response to treatment.

It is to be understood that one, some, or all of the properties of the various embodiments described herein may be combined to form other embodiments of the present invention. These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 shows a Western blot demonstrating expression of mtHSP70 (8E) and p21 in a lysate of Leishmania major amastigotes.

FIG. 2 shows immunogenicity and protection of mice immunized with the immunogenic carboxy terminal fragment of mtHSP70 (8E). A) Limiting dilution analysis of parasite burden in the livers of BALB/c mice immunized with 8E and challenged with L donovani promastigotes. B) IFNγ production from spleen cell cultures of BALB/c mice immunized with 8E, NS, 111F or saline controls and restimulated in vitro with the immunizing polypeptide (a recall response). NS and 111F serve as positive reference controls. C) IL5 production from spleen cell cultures of BALB/c mice immunized with 8E, NS, 111F or saline controls and restimulated in vitro with the immunizing polypeptide (a recall response). NS and 111F serve as positive reference controls. D) Flow cytometry analysis of spleen cell cultures from mice immunized with 8E or saline and restimulated in vitro with saline or 8E. Data is presented as the percentage of cells secreting IFNγ, IL-2, or TNF. CD4 T cells that produce multiple cytokines are termed multifunctional.

FIG. 3 shows flow cytometry analysis of spleen cell cultures from mice immunized with CxP or saline and restimulated in vitro with saline or CxP. Data is presented as the percentage of cells secreting IFNγ, IL-2, or TNF. CD4 T cells that produce multiple cytokines are termed multifunctional.

FIG. 4 shows flow cytometry analysis of spleen cell cultures from mice immunized with 821X or saline and restimulated in vitro with saline or 821X. Data is presented as the percentage of cells secreting IFNγ, IL-2, or TNF. CD4 T cells that produce multiple (two or more) cytokines are termed multifunctional.

### BRIEF DESCRIPTION OF THE SEQUENCE IDENTIFIERS

SEQ ID NO: 1 is a nucleic acid sequence encoding the 821X fusion polypeptide of SEQ ID NO: 2.

SEQ ID NO: 2 is an amino acid sequence of the 821X fusion polypeptide.

SEQ ID NO: 3 is a nucleic acid sequence encoding the 821XH fusion polypeptide of SEQ ID NO: 4.

SEQ ID NO: 4 is an amino acid sequence of the 821XH fusion polypeptide.

SEQ ID NO: 5 is a nucleic acid sequence encoding the 821XA fusion polypeptide of SEQ ID NO: 6.

SEQ ID NO: 6 is an amino acid sequence of the 821XA fusion polypeptide.

SEQ ID NO: 7 is a nucleic acid sequence encoding the 821NA fusion polypeptide of SEQ ID NO: 8.

SEQ ID NO: 8 is an amino acid sequence of the 821NA fusion polypeptide.

SEQ ID NO: 9 is a nucleic acid sequence encoding the NXH fusion polypeptide of SEQ ID NO: 10.

SEQ ID NO: 10 is an amino acid sequence of the NXH fusion polypeptide.

SEQ ID NO: 11 is a nucleic acid sequence encoding the TXL fusion polypeptide of SEQ ID NO: 12.

SEQ ID NO: 12 is an amino acid sequence of the TXL fusion polypeptide.

SEQ ID NO: 13 is a nucleic acid sequence encoding the 8XHA fusion polypeptide of SEQ ID NO: 14.

SEQ ID NO: 14 is an amino acid sequence of the 8XHA fusion polypeptide.

SEQ ID NO: 15 is a nucleic acid sequence encoding the 8NHA fusion polypeptide of SEQ ID NO: 16.

SEQ ID NO: 16 is an amino acid sequence of the 8NHA fusion polypeptide.

SEQ ID NO: 17 is a nucleic acid sequence encoding the 8CHA fusion polypeptide of SEQ ID NO: 18.

SEQ ID NO 18: is an amino acid sequence of the 8CHA fusion polypeptide.

SEQ ID NO: 19 is a nucleic acid sequence encoding the 8NCA fusion polypeptide of SEQ ID NO: 20.

SEQ ID NO: 20 is an amino acid sequence of the 8NCA fusion polypeptide.

SEQ ID NO: 21 is an amino acid sequence of a carboxy-terminal fragment of the putative mitochondrial HSP70 polypeptide (designated 8E or 8 herein) from Leishmania infantum or donovani. The 8E carboxy-terminal fragment comprises amino acids 509 to 660 of the putative mitochondrial HSP70 polypeptide.

SEQ ID NO: 22 is an amino acid sequence of a carboxy-terminal fragment of the putative mitochondrial HSP70 polypeptide (designated 8E or 8 herein) from Leishmania major.

SEQ ID NO: 23 is an amino acid sequence of a carboxy-terminal fragment of the putative mitochondrial HSP70 polypeptide (designated 8E or 8 herein) from Leishmania mexicana.

SEQ ID NO: 24 is an amino acid sequence of a carboxy-terminal fragment of the putative mitochondrial HSP70 polypeptide (designated 8E or 8 herein) from Leishmania braziliensis.

SEQ ID NO: 25 is an amino acid sequence of a full length putative carboxypeptidase polypeptide (designated CxP or X herein) from Leishmania donovani. The full length CxP polypeptide comprises amino acids 1 to 503 of the putative carboxypeptidase.

SEQ ID NO: 26 is an amino acid sequence of a full length putative carboxypeptidase polypeptide (designated CxP or X herein) from Leishmania infantum.

SEQ ID NO: 27 is an amino acid sequence of a full length putative carboxypeptidase polypeptide (designated CxP or X herein) from Leishmania major.

SEQ ID NO: 28 is an amino acid sequence of a full length putative carboxypeptidase polypeptide (designated CxP or X herein) from Leishmania mexicana.

SEQ ID NO: 29 is an amino acid sequence of a full length putative carboxypeptidase polypeptide (designated CxP or X herein) from Leishmania braziliensis.

SEQ ID NO: 30 is an amino acid sequence of a carboxy-terminal fragment of the cysteine proteinase B polypeptide (designated CpB, CPB or C herein) from Leishmania infantum. The CpB fragment comprises amino acids 154 to 443 of the cysteine proteinase B polypeptide.

SEQ ID NO: 31 is an amino acid sequence of an amino terminal fragment of the histone H2BN polypeptide (designated H2BN, h2Bn, or H herein) polypeptide from Leishmania infantum. The h2Bn amino terminal fragment comprises amino acids 1 to 46 of the histone H2BN polypeptide.

SEQ ID NO: 32 is an amino acid sequence of a mature A2 polypeptide (designated A herein) from Leishmania donovani. The mature A2 polypeptide comprises amino acids 23 to 236 of the A2 polypeptide.

SEQ ID NO: 33 is an amino acid sequence of a full length p21 antigen polypeptide (designated p21 or 21 herein) of Leishmania infantum. The 21 polypeptide comprises amino acids 1 to 191 of the p21 antigen.

SEQ ID NO: 34 is an amino acid sequence of a full length thiol specific antioxidant polypeptide (designated TSA or T herein) of Leishmania major. The TSA polypeptide comprises amino acids 1 to 199 of the thiol specific antioxidant polypeptide.

SEQ ID NO: 35 is an amino acid sequence of a putative eukaryotic initiation factor 4a polypeptide (designate Leif or L herein) of Leishmania major. The Leif polypeptide comprises amino acids 1 to 226 of the putative eukaryotic initiation factor 4a polypeptide.

SEQ ID NO: 36 is an amino acid sequence of a full length nonspecific nucleoside hydrolase polypeptide (designated NH or H herein) from Leishmania infantum/donovani. The full length polypeptide comprises amino acid 1 to 314 of the nonspecific nucleoside hydrolase polypeptide.

SEQ ID NO: 37 is an amino acid sequence of a full length A2 polypeptide (designated Afl herein) from Leishmania donovani. The Afl polypeptide comprises amino acids 1 to 236 of the A2 polypeptide.

SEQ ID NO: 38 is an amino acid sequence of Alpha Tubulin (designated T or aT herein) from Leishmania infantum. The aT polypeptide comprises amino acids 1 to 490 of Alpha Tubulin.

SEQ ID NO: 39 is an amino acid sequence of Malate Dehydrogenase (designated M or MDH herein) from Leishmania infantum. The MDH polypeptide comprises amino acids 1 to 322 of Malate Dehydrogenase.

SEQ ID NO: 40 is a nucleic acid sequence encoding the 8NC fusion polypeptide of SEQ ID NO: 41.

SEQ ID NO: 41 is an amino acid sequence of the 8NC fusion polypeptide.

SEQ ID NO: 42 is a nucleic acid sequence encoding the 8NCH fusion polypeptide of SEQ ID NO: 43.

SEQ ID NO: 43 is an amino acid sequence of the 8NCH fusion polypeptide.

SEQ ID NO: 44 is a nucleic acid sequence encoding the 8MCH fusion polypeptide of SEQ ID NO: 45.

SEQ ID NO: 45 is an amino acid sequence of the 8MCH fusion polypeptide.

SEQ ID NO: 46 is a nucleic acid sequence encoding the 8MTH fusion polypeptide of SEQ ID NO: 47.

SEQ ID NO: 47 is an amino acid sequence of the 8MTH fusion polypeptide.

SEQ ID NO: 48 is a nucleic acid sequence encoding the 8TCH fusion polypeptide of SEQ ID NO: 49.

SEQ ID NO: 49 is an amino acid sequence of the 8TCH fusion polypeptide.

### DETAILED DESCRIPTION

The practice of the present invention will employ, unless otherwise indicated, conventional techniques of molecular biology, recombinant DNA, and chemistry, which are within the skill of the art. Such techniques are explained fully in the literature. See, e.g., Molecular Cloning A Laboratory Manual, 2nd Ed., Sambrook et al., ed., Cold Spring Harbor Laboratory Press: (1989); DNA Cloning, Volumes I and II (D. N. Glover ed., 1985); Oligonucleotide Synthesis (M. J. Gait ed., 1984); Mullis et al., U.S. Pat. No. 4,683,195; Nucleic Acid Hybridization (B. D. Hames & S. J. Higgins eds. 1984); B. Perbal, A Practical Guide To Molecular Cloning (1984); the treatise, Methods In Enzymology (Academic Press, Inc., N.Y.); and in Ausubel et al., Current Protocols in Molecular Biology, John Wiley and Sons, Baltimore, Md. (1989).

As noted above, the present invention is generally directed to compositions and methods for preventing, treating and detecting leishmaniasis. The compositions of the invention include, for example, polypeptides including fusion polypeptides that comprise various immunogenic portions of Leishmania proteins, wherein the portions and variants preferably retain substantially the same or similar immunogenic properties as a corresponding full length Leishmania protein. Immunization strategies using compositions of the invention can be applied to the in vivo protection against, for example, L. infantum, L. donovani, and L. major, which are causative agents of VL in humans and dogs. The present invention also contemplates, in other embodiments, using the polypeptides including fusion polypeptides described herein in diagnostic applications, including, but not limited to, serodiagnosis and whole blood assays in patients and dogs, preferably in a format amenable to providing rapid, point of care diagnostic results, such as a lateral flow assay or a dual path platform assay.

### Leishmania Polypeptides (Including Fusion Polypeptides) and Uses Therefor

In a general aspect, the present invention provides isolated Leishmania polypeptides, as described herein, including fusion polypeptides and compositions containing the same.

In some embodiments, the invention provides an immunogenic portion of a Leishmania mitochondrial HSK70 (mtHSP70), wherein the immunogenic portion comprises a carboxy terminal region sequence of the mtHSP70 or variants thereof. In some embodiments, the carboxy terminal region sequence of the mtHSP70 comprises the amino acid sequence of SEQ ID NO: 21, 22, 23, or 24, or a sequence having at least 85% (e.g., at least 90% or at least 95%) identity to SEQ ID NO: 21, 22, 23, or 24. In some embodiments, the carboxy terminal region sequence of the mtHSP70 or variants thereof are fused with one or more immunogenic portions of another Leishmania polypeptides described herein. The invention also provides an isolated polypeptide comprises the immunogenic portion of the mtHSP70. In some embodiments, the isolated polypeptide does not contain the full length sequence of a mtHSP70.

In some embodiments, the invention provides an isolated polypeptide comprising an immunogenic portion of a Leishmania CxP or variants thereof. In some embodiments, the polypeptide comprises the amino acid sequence of SEQ ID NO: 25, 26, 27, 28, or 29, or a sequence having at least 80% (e.g., at least 90% or 95%) identity to SEQ ID NO: 25, 26, 27, 28, or 29. In some embodiments, the immunogenic portion of the CxP is fused with one or more other Leishmania polypeptides (e.g., a polypeptide described herein).

In some embodiments, polypeptides and fusion polypeptides described herein can generate an immune response or an effective immune response to Leishmania. The polypeptides and fusion polypeptides may have one or more of the following characteristics: 1) a reduction in parasite burden in immunized hosts upon experimental challenge with a Leishmania parasite infection either by direct inoculation of promastigotes or models of natural infection such as the bites of infected sandflies; 2) secretion of IFNγ in in vitro spleen cell cultures from mice immunized with the individual polypeptides or fusion polypeptides of the invention upon incubation with the matched fusion polypeptide or individual polypeptides of the fusion polypeptide; 3) IFNγ secretion in vitro spleen cell cultures from mice immunized with the individual polypeptides or fusion polypeptides of the invention following incubation with crude parasite; 4) generation of antigen-specific multifunctional Th1 cells, for example CD4 T cells that produce multiple cytokines indicative of a Th1 phenotype such as the combined production of IFNγ, TNF and IL-2 or IFNγ and TNF; and or 5) improvement or enhancement of the immune recognition of one or more individual polypeptide(s), when presented in the context of a fusion polypeptide, as measured for example by the secretion of cytokines such γIFN, or the titer of presence of antibodies or cellular responses to the polypeptide. Methods for testing one or more of the above immune responses are known in the art and are described in detail in Examples.

Different Leishmania polypeptides in the fusion polypeptides may be arranged in the fusion polypeptide in any order. For example, any particular polypeptide of the fusion polypeptide may be located towards the C-terminal end of the fusion polypeptide or the N-terminal end of the polypeptide or in the center of the fusion polypeptide (i.e., located in between at least two other polypeptides in the fusion polypeptide). Different Leishmania polypeptides may be linked by a linker sequence of any length (e.g., 2-20 amino acids).

As used herein, the term “polypeptide” or “protein” encompasses amino acid chains of any length, including full length proteins, wherein the amino acid residues are linked by covalent bonds. A polypeptide comprising an immunogenic portion of a Leishmania polypeptide or protein may consist solely of an immunogenic portion, may contain two or more immunogenic portions and/or may contain additional sequences. The additional sequences may be derived from a native Leishmania polypeptide or protein or may be heterologous, and such heterologous sequences may (but need not) be immunogenic.

An “isolated polypeptide” is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. One of ordinary skill in the art would appreciate that antigenic polypeptide fragments could also be obtained from those already available in the art. Polypeptides of the invention, antigenic/immunogenic fragments thereof, and other variants may be prepared using conventional recombinant and/or synthetic techniques.

The Leishmania polypeptide used in a polypeptide or a fusion polypeptide of the present invention can be full length, substantially full length polypeptides, or variants thereof as described herein. Alternatively, a fusion polypeptide or composition of the invention can comprise or consist of immunogenic portions or fragments of a full length Leishmania polypeptide, or variants thereof.

In certain embodiments, an immunogenic portion of a Leishmania polypeptide is a portion that is capable of eliciting an immune response (i.e., cellular and/or humoral) in a presently or previously Leishmania-infected patient (such as a human or a mammal (e.g., a dog)) and/or in cultures of lymph node cells or peripheral blood mononuclear cells (PBMC) isolated from presently or previously Leishmania-infected individuals. The cells in which a response is elicited may comprise a mixture of cell types or may contain isolated component cells (including, but not limited to, T-cells, NK cells, macrophages, monocytes and/or B cells). In a particular embodiment, immunogenic portions of a fusion polypeptide of the invention are capable of inducing T-cell proliferation and/or a predominantly Th1-type cytokine response (e.g., IL-2, IFN-y, and/or TNFα production by T-cells and/or NK cells, and/or IL-12 production by monocytes, macrophages and/or B cells). Immunogenic portions of the antigens described herein may generally be identified using techniques known to those of ordinary skill in the art, including the representative methods summarized in Paul, Fundamental Immunology, 5th ed., Lippincott Williams & Wilkins, 2003 and references cited therein. Such techniques include screening fusion polypeptides for the ability to react with antigen-specific antibodies, antisera and/or T cell lines or clones. As used herein, antisera and antibodies are “antigen-specific” if they specifically bind to an antigen (i.e., they react with the protein in an immunoassay, and do not react detectably with unrelated proteins). Such antisera and antibodies may be prepared as described herein and using well-known techniques.

Immunogenic portions of a Leishmania can be essentially any length; provided they retain one or more of the immunogenic regions that are responsible for or contribute to the in vivo protection provided against leishmaniasis by one or more fusion polypeptides of the invention, as disclosed herein. In one embodiment, the ability of an immunogenic portion to react with antigen-specific antisera may be enhanced or unchanged, relative to the native protein, or may be diminished by less than 50%, and preferably less than 20%, relative to the native protein. Illustrative portions will generally be at least 10, 15, 25, 50, 150, 200, 250, 300, or 350 amino acids in length, or more, up to and including full length Leishmania polypeptide.

In some embodiments, a Leishmania polypeptide or protein described herein includes a mtHSP70 polypeptide, a CxP polypeptide, a CpB polypeptide, a h2BN polypeptide, an A2 polypeptide, a TSA polypeptide, a Leif polypeptide, a NH polypeptide, a MDH polypeptide, and an AT polypeptide. In some embodiments, the Leishmania polypeptide or protein is from a L. infantum, a L. donovani, a L. major, a L. mexicana, or a L. braziliensis strain. In some embodiments, the fusion polypeptide comprises sequence s from at least two, at least three, at least four different Leishmania strains. In some embodiments, these Leishmania polypeptides (including immunogenic portions) include any naturally occurring variants.

In a particular embodiment, immunogenic portions of a Leishmania polypeptide are those, which when used in combination, are capable of providing protection against, for example in an in vivo assay as described herein, or serodiagnosis of Leishmania species such as L. donovani, L. major and/or L. infantum, which are believed to be causative agents of VL in humans and dogs. In addition, polypeptides (including fusion polypeptides) of the invention may also be useful in blocking transmission of the causative agent of VL from dogs to humans, e.g., by reducing or eliminating the number of parasites in the blood and skin of infected dogs.

As would be recognized by the skilled artisan, a polypeptide composition of the invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof. In a specific embodiment, the polypeptide is a fusion polypeptide, as described herein.

As noted, in various embodiments of the present invention, fusion polypeptides generally comprise at least an immunogenic portion or variant of the Leishmania polypeptides described herein. In some instances, preferred immunogenic portions will be identified that have a level of immunogenic activity greater than that of the corresponding full-length polypeptide, e.g., having greater than about 100% or 150% or more immunogenic activity. In particular embodiments, the immunogenicity of the full-length fusion polypeptide will have additive, or greater than additive immunogenicity contributed by of each of the antigenic/immunogenic portions contained therein.

In another aspect, fusion polypeptides of the present invention may contain multiple copies of polypeptide fragments, repeats of polypeptide fragments, or multimeric polypeptide fragments, including antigenic/immunogenic fragments, such as Leishmania polypeptides comprising at least about 1, 2, 3, 4, 5, 6, 7, 8, 9, 10 or more contiguous fragments of a Leishmania polypeptide, in any order, and including all lengths of a polypeptide composition set forth herein, or those encoded by a polynucleotide sequence set forth herein.

In some embodiments, the immunogenic portion of a mtHSP70 polypeptide comprises the amino acid sequence of SEQ ID NO: 21, 22, 23, or 24, or a sequence having at least 85% identity (e.g., at least 90% or at least 95%) to SEQ ID NO: SEQ ID NO: 21, 22, 23, or 24. In some embodiments, a CxP polypeptide comprises the amino acid sequence of SEQ ID NO: 25, 26, 27, 28, or 29, or a sequence having at least 85% identity (e.g., at least 90% or at least 95%) to SEQ ID NO: SEQ ID NO: 25, 26, 27, 28, or 29. In some embodiments, the immunogenic portion of a CpB comprises the amino acid sequence of SEQ ID NO: 30, or a sequence having at least 85% identity (e.g., at least 90% or at least 95%) to SEQ ID NO: 30. In some embodiments, the immunogenic portion of a H2BN comprises the amino acid sequence of SEQ ID NO: 31, or a sequence having at least 85% identity (e.g., at least 90% or at least 95%) to SEQ ID NO: 31. In some embodiments, an A2 polypeptide comprises the amino acid sequence of SEQ ID NO: 32 or 37, or a sequence having at least 85% identity (e.g., at least 90% or at least 95%) to SEQ ID NO: 32 or 37. In some embodiments, a p21 polypeptide comprises the amino acid sequence of SEQ ID NO: 33, or a sequence having at least 85% identity (e.g., at least 90% or at least 95%) to SEQ ID NO: 33. In some embodiments, a TSA polypeptide comprises the amino acid sequence of SEQ ID NO: 34, or a sequence having at least 85% identity (e.g., at least 90% or at least 95%) to SEQ ID NO: 34. In some embodiments, a LeiF polypeptide comprises the amino acid sequence of SEQ ID NO: 35 or a sequence having at least 85% identity (e.g., at least 90% or at least 95%) to SEQ ID NO: 35. In some embodiments, a NH polypeptide comprises the amino acid sequence of SEQ ID NO: 36 or a sequence having at least 85% identity (e.g., at least 90% or at least 95%) to SEQ ID NO: 36. In some embodiments, a NH polypeptide comprises an immunogenic portion of the amino acid sequence of SEQ ID NO:1, 3, or 5 or an amino acid sequence having at least 85% identity (e.g., at least 90% or at least any of 95%, 96%, 97%, 98%, and 99%) to SEQ ID NO:1, 3, or 5 as disclosed in U.S. Pub. No. 2012/0114688, which is incorporated herein by reference. In some embodiments, a aT polypeptide comprises the amino acid sequence of SEQ ID NO: 38 or a sequence having at least 85% identity (e.g., at least 90% or at least 95%) to SEQ ID NO: 38. In some embodiments, a MDH polypeptide comprises the amino acid sequence of SEQ ID NO: 39 or a sequence having at least 85% identity (e.g., at least 90% or at least 95%) to SEQ ID NO: 39.

In another aspect, the invention provides a fusion polypeptide comprising, consisting of, or consisting essentially of the amino acid sequence set forth in SEQ ID NO: 2, 4, 6, 8, 10, 12, 14, 16, 18, 20, 41, 43, 45, 47, or 49, or a sequence having at least 85%, at least 90%, at least 95% or at least 98% identity thereto.

As one of ordinary skill in the art would understand the fusion polypeptides described herein may contain an optional amino terminal linker comprising a methionine initiation codon and six histidine amino acids (his tag) encoded by the polynucleotides 5′-ATGCATCAC CATCAC CATCAC3′ (SEQ ID NO:50) immediately 5′ to the initiation methionine encoded by ATG codon of the fusion polypeptide. For fusion polypeptides wherein the first polypeptide is the carboxy terminus of the putative mtHSP70 poly peptide, 8 or 8E, the his tagged fusion polynucleotide does not comprise a 3′ ATG codon prior to the first polynucleotide encoding 8E.

In yet another aspect, the present invention provides fusion polypeptides comprising one or more variants of the Leishmania polypeptide (including immunogenic portions) described herein. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequence set forth herein.

In other related embodiments, a polypeptide “variant,” includes polypeptides that differ from a native protein in one or more substitutions, deletions, additions and/or insertions, such that the desired immunogenicity of the variant polypeptide is not substantially diminished relative to a native polypeptide.

For example, certain variants of the invention include polypeptides of the invention that have been modified to replace one or more cysteine residues with alternative residues. Such polypeptides are referred to hereinafter as cysteine-modified polypeptides or cysteine-modified fusion polypeptides. Preferably, the modified polypeptides retain substantially the same or similar immunogenic properties as the corresponding unmodified polypeptides. In a more specific embodiment, cysteine residues are replaced with serine residues because of the similarity in the spatial arrangement of their respective side chains. However, it will be apparent to one skilled in the art that any amino acid that is incapable of interchain or intrachain disulfide bond formation can be used as a replacement for cysteine. When all or substantially all of the cysteine residues in a polypeptide or fusion polypeptide of this invention are replaced, the resulting cysteine-modified variant may be less prone to aggregation and thus easier to purify, more homogeneous, and/or obtainable in higher yields following purification.

In one embodiment, the ability of a variant to react with antigen-specific antisera may be enhanced or unchanged, relative to the native protein, or may be diminished by less than 50%, and preferably less than 20%, relative to a corresponding native or control polypeptide. In a particular embodiment, a variant of an Leishmania polypeptide is one capable of providing protection, for example in an in vivo assay as described herein, against a Leishmania species such as L. donovani, L. infantum and/or L. major.

In particular embodiments, a fusion polypeptide of the present invention comprises at least 1, at least 2, at least 3, at least 4, at least 5, at least 6, at least 7, at least 8, at least 9, or at least 10 or more substitutions, deletions, additions and/or insertions within a Leishmania polypeptide, where the fusion polypeptide is capable of providing protection against, for example in an in vivo assay as described herein, Leishmania species such as L. donovani, L. major and/or L. infantum.

In related embodiments, a fusion polypeptide of the present invention comprises at least 1, at least 2, at least 3, at least 4, at least 5, at least 6, at least 7, at least 8, at least 9, or at least 10 or more substitutions, deletions, additions and/or insertions within a Leishmania polypeptide, where the fusion polypeptide is capable of serodiagnosis of Leishmania species such as L. donovani, L. major and/or L. infantum.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, immunogenic variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| | Amino | | |
|---|---|---|---|
| | Acids | | Codons |
| Alanine | Ala | A | GCA GCC GCG GCU |
| Cysteine | Cys | C | UGC UGU |
| Aspartic acid | Asp | D | GAG GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-Histidine tag (6×His), GST, MBP, TAP/TAG, FLAG epitope, MYC epitope, V5 epitope, VSV-G epitope, etc.), or to enhance binding of the polypeptide to a solid support. For example, a polypeptide may be conjugated to an immunoglobulin Fc region.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Alignment of sequences for comparison may be conducted using, for example, the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626 [symbol] 645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) MoL Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, alignment of sequences for comparison may be conducted by the local identity algorithm of Smith and Waterman (1981) Add. APL. Math 2:482, by the identity alignment algorithm of Needleman and Wunsch (1970) J. MoL Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M=5, N=−4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, as noted above, the present invention encompasses polynucleotide and polypeptide sequences having substantial identity to the sequences disclosed herein, for example those comprising at least 70%, 75%, 80%, 85%, 90%, 95%, 96%, 97%, 98%, or 99% or higher, sequence identity compared to a polynucleotide or polypeptide sequence of this invention (e.g., as set out in SEQ ID NOs:1-49) using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like. Furthermore, it would be understood by of ordinary skill in the art that fusion polypeptides of the present invention may comprise at least 2, at least 3, or at least 4 or more antigenic/immunogenic portions or fragments of a polypeptide comprising at least 70%, 75%, 80%, 85%, 90%, 95%, 96%, 97%, 98%, or 99% or higher, sequence identity to a Leishmania polypeptide that is capable of providing protection against, for example in an in vivo assay as described herein, or serodiagnosis of Leishmania species such as L. donovani, L. major and/or L. infantum.

In another aspect of the invention, fusion polypeptides are provided that comprise at least an immunogenic portion of a polypeptide and further comprise a heterologous fusion partner, as well as polynucleotides encoding such fusion polypeptides. For example, in one embodiment, a fusion polypeptide comprises one or more immunogenic portions or fragments of a Leishmania polypeptide and one or more additional immunogenic Leishmania sequences, which are joined via a peptide linkage into a single amino acid chain.

In another embodiment, a fusion polypeptide may comprise multiple Leishmania antigenic portions. In some embodiments, at least one of the portions in the fusion polypeptide is from a Leishmania mhHSP70 or CxP polypeptide. In some embodiments, an immunogenic portion is a portion of an antigen that reacts with blood samples from Leishmania-infected individuals (i.e. an epitope is specifically bound by one or more antibodies and/or T-cells present in such blood samples.

In certain embodiments, a fusion polypeptide may further comprise at least one heterologous fusion partner having a sequence that assists in providing T helper epitopes (an immunological fusion partner), preferably T helper epitopes recognized by humans, or that assists in expressing the protein (an expression enhancer) at higher yields than the native recombinant protein. Certain preferred fusion partners include both immunological and expression-enhancing fusion partners. Other fusion partners may be selected so as to increase the solubility of the protein or to enable the protein to be targeted to desired intracellular compartments. Still further fusion partners include affinity tags, such as V5, 6×HIS, MYC, FLAG, and GST, which facilitate purification of the protein. It would be understood by one having ordinary skill in the art that those unrelated sequences may, but need not, be present in a fusion polypeptide used in accordance with the present invention. Within a particular embodiment, an immunological fusion partner comprises sequence derived from protein D, a surface protein of the gram-negative bacterium Haemophilus influenza B (WO 91/18926). For example, one protein D derivative comprises approximately the first third of the protein (e.g., the first N-terminal 100 110 amino acids), and a protein D derivative may be lipidated. Within certain embodiments, the first 109 residues of a lipoprotein D fusion partner is included on the N-terminus to provide the polypeptide with additional exogenous T cell epitopes and to increase the expression level in E. coli (thus functioning as an expression enhancer). The lipid tail ensures optimal presentation of the antigen to antigen presenting cells. Other illustrative fusion partners include the non-structural protein from influenzae virus, NS1 (hemaglutinin). Typically, the N-terminal 81 amino acids are used, although different fragments that include T-helper epitopes may also be used.

In another particular embodiment, an immunological fusion partner comprises an amino acid sequence derived from the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292 (1986)). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798 (1992)). Within a particular embodiment, a repeat portion of LYTA may be incorporated into a fusion protein. A repeat portion is found in the C-terminal region starting at residue 178. A more particular repeat portion incorporates residues 188-305.

Fusion sequences may be joined directly (i.e., with no intervening amino acids) or may be joined by way of a linker sequence (e.g., Gly-Cys-Gly) that does not significantly diminish the immunogenic properties of the component polypeptides. The polypeptides forming the fusion protein are typically linked C-terminus to N-terminus, although they can also be linked C-terminus to C-terminus, N-terminus to N-terminus, or N-terminus to C-terminus. The polypeptides of the fusion protein can be in any order. Fusion polypeptides or fusion proteins can also include conservatively modified variants, polymorphic variants, alleles, mutants, subsequences, interspecies homologs, and immunogenic fragments of the antigens that make up the fusion protein.

Fusion polypeptides may generally be prepared using standard techniques, including recombinant technology, chemical conjugation and the like. For example, DNA sequences encoding the polypeptide components of a fusion may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in frame. This permits translation into a single fusion polypeptide that retains or in some cases exceeds the biological activity of the component polypeptides.

A peptide linker sequence may be employed to separate the fusion components by a distance sufficient to ensure that each polypeptide folds into its desired secondary and/or tertiary structures. Such a peptide linker sequence may be incorporated into the fusion polypeptide using standard techniques well known in the art. Suitable peptide linker sequences may be chosen, for example, based on one or more of the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Certain preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

In addition to recombinant fusion polypeptide expression, Leishmania polypeptides, immunogenic portions, variants and fusions thereof may be generated by synthetic or recombinant means. Synthetic polypeptides having fewer than about 100 amino acids, and generally fewer than about 50 amino acids, may be generated using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain (Merrifield, J. Am. Chem. Soc. 85:2149-2146, 1963). Equipment for automated synthesis of polypeptides is commercially available from suppliers such as Perkin Elmer/Applied BioSystems Division, Foster City, Calif., and may be operated according to the manufacturer's instructions. Thus, for example, Leishmania antigens, or portions thereof, may be synthesized by this method.

Recombinant polypeptides containing portions and/or variants of a native Leishmania polypeptide may be readily prepared from a DNA sequence encoding the antigen, using well known and established techniques. In particular embodiments, a fusion polypeptide comprising Leishmania antigens may be readily prepared from a DNA sequence encoding the cloned fused antigens. For example, supernatants from suitable host/vector systems which secrete recombinant protein into culture media may be first concentrated using a commercially available filter. Following concentration, the concentrate may be applied to a suitable purification matrix such as an affinity matrix, a size exclusion chromatography matrix or an ion exchange resin.

Alternatively, any of a variety of expression vectors known to those of ordinary skill in the art may be employed to express recombinant polypeptides of this invention. Expression may be achieved in any appropriate host cell that has been transformed or transfected with an expression vector containing a polynucleotide that encodes a recombinant polypeptide. Preferably, the host cells are E. coli, yeast, an insect cell line (such as Spodoptera or Trichoplusia) or a mammalian cell line, including (but not limited to) CHO, COS, HEK-293T and NS-1. The DNA sequences expressed in this manner may encode naturally occurring proteins, and fusion proteins comprising Leishmania antigens, such as those described herein, portions thereof, and repeats or other variants of such proteins. Expressed fusion polypeptides of this invention are generally isolated in substantially pure form. Preferably, the fusion polypeptides are isolated to a purity of at least 80% by weight, more preferably, to a purity of at least 95% by weight, and most preferably to a purity of at least 99% by weight. In general, such purification may be achieved using, for example, the standard techniques of ammonium sulfate fractionation, SDS-PAGE electrophoresis, and affinity chromatography.

Leishmania polypeptides and polynucleotides of the invention may be prepared or isolated using any of a variety of procedures and using any of a variety of Leishmania species including, but not limited to, L. donovani, L. chagasi, L. infantum, L. major, L. amazonensis, L. braziliensis, L. panamensis, L. mexicana, L. tropics, and L. guyanensis. Such species are available, for example, from the American Type Culture Collection (ATCC), Rockville, Md.

Regardless of the method of preparation, the polypeptides or fusion polypeptides produced as described above are preferably immunogenic. In certain embodiments, for example, the polypeptides (or immunogenic portions thereof) are capable of eliciting an immune response in cultures of lymph node cells and/or peripheral blood mononuclear cells (PBMC) isolated from presently or previously Leishmania-infected individuals. More specifically, in certain embodiments, the antigens, and immunogenic portions thereof, have the ability to induce T-cell proliferation and/or to elicit a dominantly Th1-type cytokine response (e.g., IL-2, IFN-y, and/or TNF-a production by T-cells and/or NK cells; and/or IL-12 production by monocytes, macrophages and/or B cells) in cells isolated from presently or previously Leishmania-infected individuals. A Leishmania-infected individual may be afflicted with a form of leishmaniasis (such as subclinical, cutaneous, mucosal or active visceral) or may be asymptomatic. Such individuals may be identified using methods known to those of ordinary skill in the art. Individuals with leishmaniasis may be identified based on clinical findings associated with, for example, at least one of the following: isolation of parasite from lesions, a positive skin test with Leishmania lysate or a positive serodiagnostic test. Asymptomatic individuals are infected individuals who have no signs or symptoms of the disease. Such individuals can be identified, for example, based on a positive serological test and/or skin test with Leishmania lysate.

The term “PBMC,” which refers to a preparation of nucleated cells consisting primarily of lymphocytes and monocytes that are present in peripheral blood, encompasses both mixtures of cells and preparations of one or more purified cell types. PBMC may be isolated by methods known to those in the art. For example, PBMC may be isolated by density centrifugation through, for example, Ficoll™ (Winthrop Laboratories, New York). Lymph node cultures may generally be prepared by immunizing BALB/c mice (e.g., in the rear foot pad) with Leishmania promastigotes emulsified in complete Freund's adjuvant. The draining lymph nodes may be excised following immunization and T-cells may be purified in an anti-mouse Ig column to remove the B cells, followed by a passage through a Sephadex G10 column to remove the macrophages. Similarly, lymph node cells may be isolated from a human following biopsy or surgical removal of a lymph node.

The ability of a fusion polypeptide of the invention to induce a response in PBMC or lymph node cell cultures may be evaluated, for example, by contacting the cells with the polypeptide and measuring a suitable response. In general, the amount of polypeptide that is sufficient for the evaluation of about 2×10⁵ cells ranges from about 10 ng to about 100 jig or 100 ng to about 50 ug, and preferably is about 1 ug, to 10 ug. The incubation of polypeptide (e.g., a fusion polypeptide) with cells is typically performed at 37° C. for about 1-3 days. Following incubation with polypeptide, the cells are assayed for an appropriate response. If the response is a proliferative response, any of a variety of techniques well known to those of ordinary skill in the art may be employed. For example, the cells may be exposed to a pulse of radioactive thymidine and the incorporation of label into cellular DNA measured. In general, a polypeptide that results in at least a three fold increase in proliferation above background (i.e., the proliferation observed for cells cultured without polypeptide) is considered to be able to induce proliferation.

Alternatively, the response to be measured may be the secretion of one or more cytokines (such as interferon-y (IFN-y), interleukin-4 (IL-4), interleukin-12 (p70 and/or p40), interleukin-2 (IL-2) and/or tumor necrosis factor-a (TNF-a)) or the change in the level of mRNA encoding one or more specific cytokines. For example, the secretion of interferon-y, interleukin-2, tumor necrosis factor-a and/or interleukin-12 is indicative of a Th1 response, which contributes to the protective effect against Leishmania. Assays for any of the above cytokines may generally be performed using methods known to those of ordinary skill in the art, such as an enzyme-linked immunosorbent assay (ELISA). Suitable antibodies for use in such assays may be obtained from a variety of sources such as Chemicon, Temucula, Calif. and PharMingen, San Diego, Calif., and may generally be used according to the manufacturer's instructions. The level of mRNA encoding one or more specific cytokines may be evaluated by, for example, amplification by polymerase chain reaction (PCR). In general, a polypeptide that is able to induce, in a preparation of about 1-3×10⁵ cells, the production of 30 pg/mL of IL-12, IL-4, IFN-y, TNF-a or IL-12 p40, or 10 pg/mL of IL-12 p70, is considered able to stimulate production of a cytokine.

### Polynucleotide Compositions

The present invention also provides isolated polynucleotides, particularly those encoding the polypeptide combinations and/or fusion polypeptides of the invention, as well as compositions comprising such polynucleotides. As used herein, the terms “DNA” and “polynucleotide” and “nucleic acid” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the polynucleotide sequences of this invention can include genomic sequences, extra-genomic and plasmid-encoded sequences and smaller engineered gene segments that express, or may be adapted to express, proteins, fusion polypeptides, peptides and the like. Such segments may be naturally isolated, recombinant, or modified synthetically by the hand of man.

As will be recognized by the skilled artisan, polynucleotides may be single-stranded (coding or antisense) or double-stranded, and may be DNA (genomic, cDNA or synthetic) or RNA molecules. Any polynucleotide may be further modified to increase stability in vivo. Possible modifications include, but are not limited to, the addition of flanking sequences at the 5′ and/or 3′ ends; the use of phosphorothioate or 2′ 0-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes a Leishmania antigen or a portion thereof) or may comprise a variant, or a biological or antigenic functional equivalent of such a sequence. In particular embodiments, polynucleotides may encode for two or more antigenic/immunogenic portions, fragments, or variants derived from the Leishmania antigens described herein. In some embodiments, polynucleotides of the present invention comprise a sequence encoding any of the immunogenic portions described herein. In some embodiments, the polynucleotide comprises the sequence of SEQ ID NO: 1, 3, 5, 7, 9, 11, 13, 15, 17, 19, 40, 42, 44, 46, or 48. Of course, portions of these sequences and variant sequences sharing identity to these sequences may also be employed (e.g., those having at least about any of 80%, 85%, 90%, 91%, 92%, 93%, 94%, 95%, 96%, 97%, 98%, or 99% thereto).

Polynucleotide variants may contain one or more substitutions, additions, deletions and/or insertions, as further described below, preferably such that the immunogenicity of the encoded polypeptide is not diminished, relative to the native protein. The effect on the immunogenicity of the encoded polypeptide may generally be assessed as described herein.

For example, in certain embodiments, variants of the invention include cysteine-modified polynucleotides in which the cysteine-encoding codons are replaced with codons encoding other amino acids not capable of forming intrachain or interchain disulfide bonds. In more specific embodiments, some or all of the replacement codons encode serine because of the spatial similarity of the serine sidechain to the cysteine sidechain in the resulting polypeptide. In another specific embodiment, some or all of the replacement codons encode alanine. Illustrative methods of replacing cysteine and other codons within a polynucleotide are well known (e.g., U.S. Pat. No. 4,816,566, the contents of which are incorporated herein by reference, and Proc Natl Acad Sci 97 (15): 8530, 2000).

The term “variants” also encompasses homologous genes of xenogenic origin.

In additional embodiments, isolated polynucleotides of the present invention comprise various lengths of contiguous stretches of sequence identical to or complementary to the sequence encoding Leishmania polypeptides, such as those sequences disclosed herein. For example, polynucleotides are provided by this invention that comprise at least about 15, 20, 30, 40, 50, 75, 100, 150, 200, 300, 400, 500 or 1000 or more contiguous nucleotides of two or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a polynucleotide fragment of almost any length may be employed; with the total length preferably being limited by the ease of preparation and use in the intended recombinant DNA protocol.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention, for example polynucleotides that are optimized for human and/or primate codon selection. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Leishmania polynucleotides and fusions thereof may be prepared, manipulated and/or expressed using any of a variety of well established techniques known and available in the art. In particular embodiments, fusions comprise two or more polynucleotide sequences encoding Leishmania polypeptides.

For example, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide of the present invention.

As will be understood by those of skill in the art, it may be advantageous in some instances to produce fusion polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter fusion polypeptide encoding sequences for a variety of reasons, including but not limited to, alterations which modify the cloning, processing, expression and/or immunogenicity of the gene product.

In order to express a desired fusion polypeptide comprising two or more antigenic/immunogenic fragments or portions of Leishmania polypeptides, a nucleotide sequence encoding the fusion polypeptide, or a functional equivalent, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook et al., Molecular Cloning, A Laboratory Manual (2001), and Ausubel et al., Current Protocols in Molecular Biology (January 2008, updated edition).

A variety of expression vector/host systems are known and may be utilized to contain and express polynucleotide sequences. These include, but are not limited to, microorganisms such as bacteria transformed with recombinant bacteriophage, plasmid, or cosmid DNA expression vectors; yeast (such as Saccharomyces or Pichia) transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORTI plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, vectors which direct high level expression of fusion proteins that are readily purified may be used. Such vectors include, but are not limited to, the multifunctional E. coli cloning and expression vectors such as PBLUESCRIPT (Stratagene), in which the sequence encoding the polypeptide of interest may be ligated into the vector in frame with sequences for the amino-terminal Met and the subsequent 7 residues of B-galactosidase so that a hybrid protein is produced; pIN vectors (Van Heeke & Schuster, J. Biol. Chem. 264:5503 5509 (1989)); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al., Methods Enzymol. 153:516-544 (1987).

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, EMBO J. 6:307-311 (1987)). Alternatively, plant promoters such as the small subunit of RUBISCO or heat shock promoters may be used (Coruzzi et EMBO J. 3:1671-1680 (1984); Broglie et al., Science 224:838-843 (1984); and Winter et al., Results Probl. Cell Differ. 17:85-105 (1991)). These constructs can be introduced into plant cells by direct DNA transformation or pathogen-mediated transfection. Such techniques are described in a number of generally available reviews (see, e.g., Hobbs in McGraw Hill, Yearbook of Science and Technology, pp. 191-196 (1992)).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard et al., Proc. Natl. Acad. Sci. U.S.A. 91:3224-3227 (1994)).

In mammalian host cells, a number of viral-based expression systems are generally available. For example, in cases where an adenovirus is used as an expression vector, sequences encoding a polypeptide of the present invention may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan & Shenk, Proc. Natl. Acad. Sci. U.S.A. 81:3655-3659 (1984)). In addition, transcription enhancers, such as the Rous sarcoma virus (RSV) enhancer, may be used to increase expression in mammalian host cells.

Specific initiation signals may also be used to achieve more efficient translation of sequences encoding a fusion polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf. et al., Results ProbL Cell Differ. 20:125-162 (1994)).

In addition, a host cell strain may be chosen for its ability to modulate the expression of the inserted sequences or to process the expressed fusion protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and W138, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a fusion polynucleotide of the present invention may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler et al., Cell 11:223-232 (1977)) and adenine phosphoribosyltransferase (Lowy et al., Cell 22:817-823 (1990)) genes which can be employed in tk− or aprt− cells, respectively. Also, antimetabolite, antibiotic or herbicide resistance can be used as the basis for selection; for example, dhfr which confers resistance to methotrexate (Wigler et al., Proc. Natl. Acad. Sci. U.S.A. 77:3567-70 (1980)); npt, which confers resistance to the aminoglycosides, neomycin and G-418 (Colbere-Garapin et al., J. Mol. Biol. 150:1-14 (1981)); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman & Mulligan, Proc. Natl. Acad. Sci. U.S.A. 85:8047-51 (1988)). The use of visible markers has gained popularity with such markers as anthocyanins, B-glucuronidase and its substrate GUS, and luciferase and its substrate luciferin, being widely used not only to identify transformants, but also to quantify the amount of transient or stable protein expression attributable to a specific vector system (Rhodes et al., Methods MoL Biol. 55:121-131 (1995)).

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). These and other assays are described, among other places, in Hampton et al., Serological Methods, a Laboratory Manual (1990) and Maddox et al., J. Exp. Med. 158:1211-1216 (1983).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used, include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. In addition to recombinant production methods, fusion polypeptides of the invention, and fragments thereof, may be produced by direct peptide synthesis using solid-phase techniques (Merrifield, J. Am. Chem. Soc. 85:2149-2154 (1963)). Protein synthesis may be performed using manual techniques or by automation. Automated synthesis may be achieved, for example, using Applied Biosystems 431A Peptide Synthesizer (Perkin Elmer). Alternatively, various fragments, for example, immunogenic fragments from Leishmania polypeptides, may be chemically synthesized separately and combined using chemical methods to produce the full length molecule.

### Pharmaceutical and Vaccine Compositions

In certain aspects, the polypeptides, polynucleotides, portions, variants, fusion polypeptides, etc., as described herein, are incorporated into pharmaceutical compositions or vaccines. Pharmaceutical compositions generally comprise one or more polypeptides, polynucleotides, portions, variants, fusion polypeptides, etc., as described herein, in combination with a physiologically acceptable carrier. Vaccines, also referred to as immunogenic compositions, generally comprise one or more of the polypeptides, polynucleotides, portions, variants, fusion proteins, etc., as described herein, in combination with an immunostimulant, such as an adjuvant. In particular embodiments, the pharmaceutical compositions comprise fusion polypeptides containing Leishmania antigens (or portions or variants thereof) that are capable of providing protection against, for example in an in vivo assay as described herein, Leishmania species such as L. donovani, L. major and/or L. infantum.

An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see, e.g., Fullerton, U.S. Pat. No. 4,235,877). Vaccine preparation is generally described in, for example, Powell & Newman, eds., Vaccine Design (the subunit and adjuvant approach) (1995).

Any of a variety of immunostimulants may be employed in the vaccines of this invention. For example, an adjuvant may be included. Many adjuvants contain a substance designed to protect the antigen from rapid catabolism, such as aluminum hydroxide or mineral oil, and a stimulator of immune responses, such as lipid A (natural or synthetic), Bordatella pertussis or Mycobacterium species or Mycobacterium-derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 and derivatives thereof (GlaxoSmithKline Beecham, Philadelphia, Pa.); CWS, TDM, LeIF, aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quit A. Cytokines, such as GM-CSF or interleukin-2, -7, or -12, may also be used as adjuvants.

Certain embodiments of the present invention contemplate vaccine and pharmaceutical compositions that include one or more toll-like receptor agonists (TLR agonist). In more specific embodiments, for example, the compositions of the invention include Toll-like receptor agonists, such as TLR7 agonists and TLR7/8 agonists. In certain embodiments the TLR agonist is capable of delivering a biological signal by interacting with at least one TLR that is selected from TLR-2, TLR-3, TLR-4, TLR-5, TLR-6, TLR-7, TLR-8 and TLR-9.

Toll-like receptors (TLR) include cell surface transmembrane receptors of the innate immune system that confer early-phase recognition capability to host cells for a variety of conserved microbial molecular structures such as may be present in or on a large number of infectious pathogens. (e.g., Armant et al., 2002 Genome Biol. 3(8):reviews3011.1-3011.6; Fearon et al., 1996 Science 272:50; Medzhitov et al., 1997 Curr. Opin. lmmunol. 9:4; Luster 2002 Curr. Opin. Immunol. 14:129; Lien et al. 2003 Nat. Immunol. 4:1162; Medzhitov, 2001 Nat. Rev. Immunol. 1:135; Takeda et al., 2003 Ann Rev Immunol. 21:335; Takeda et al. 2005 Inf. Immunol. 17:1; Kaisho et al., 2004 Microbes Infect. 6:1388; Datta et al., 2003 J. Immunol. 170:4102).

Induction of TLR-mediated signal transduction to potentiate the initiation of immune responses via the innate immune system may be effected by TLR agonists, which engage cell surface TLR or cytoplasmic TLR. For example, lipopolysaccharide (LPS) may be a TLR agonist through TLR2 or TLR4 (Tsan et al., 2004 J. Leuk. Biol. 76:514; Tsan et al., 2004 Am. J. Physiol. Cell Phsiol. 286:C739; Lin et al., 2005 Shock 24:206); poly(inosine-cytidine) (polyl:C) may be a TLR agonist through TLR3 (Salem et al., 2006 Vaccine 24:5119); CpG sequences (oligodeoxynucleotides containing unmethylated cytosine-guanosine or “CpG” dinucleotide motifs, e.g., CpG 7909, Cooper et al., 2005 AIDS 19:1473; CpG 10101 Bayes et al. Methods Find Exp Clin Pharmacol 27:193; Vollmer et al. Expert Opinion on Biological Therapy 5:673; Vollmer et al., 2004 Antimicrob. Agents Chemother. 48:2314; Deng et al., 2004 J. Immunol. 173:5148) may be TLR agonists through TLR9 (Andaloussi et a., 2006 Glia 54:526; Chen et al., 2006 J. Immunol. 177:2373); peptidoglycans may be TLR2 and/or TLR6 agonists (Soboll et al., 2006 Biol. Reprod. 75:131; Nakao et al., 2005 J. Immunol. 174:1566); 3M003 (4-amino-2-(ethoxymethyl)-a,a-dimethyl-617,8,9-tetrahydro-1H-imidazo[4,5-c]quinoline-1-ethanol hydrate, Mol. Wt. 318 Da from 3M Pharmaceuticals, St. Paul, Minn., which is also a source of the related compounds 3M001 and 3M002; Gorden et al., 2005 J. Immunol. 174:1259) may be a TLR7 agonist (Johansen 2005 Clin. Exp. Allerg. 35:1591) and/or a TLR8 agonist (Johansen 2005); flagellin may be a TLRS agonist (Feuillet et al., 2006 Proc. Nat. Acad. Sci. USA 103:12487); and hepatitis C antigens may act as TLR agonists through TLR7 and/or TLR9 (Lee et al., 2006 Proc. Nat. Acad. Sci. USA 103:1828; Horsmans et al., 2005 Hepatol. 42:724). Other TLR agonists are known (e.g., Schirmbeck et al., 2003 J. Immunol. 171:5198) and may be used according to certain of the presently described embodiments.

For example, and by way of background (see, e.g., U.S. Pat. No. 6,544,518) immunostimulatory oligonucleotides containing ummethylated CpG dinucleotides (“CpG”) are known as being adjuvants when administered by both systemic and mucosal routes (WO 96/02555, EP 468520, Davis et al., J. lmmunol, 1998. 160(2):870-876; McCluskie and Davis, J. Immunol., 1998, 161(9):4463-6). CpG is an abbreviation for cytosine-guanosine dinucleotide motifs present in DNA. The central role of the CG motif in immunostimulation was elucidated by Krieg, Nature 374, p 546 1995. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may be used in certain embodiments of the present invention. CpG when formulated into vaccines, may be administered in free solution together with free antigen (WO 96/02555; McCluskie and Davis, supra) or covalently conjugated to an antigen (PCT Publication No. WO 98/16247), or formulated with a carrier such as aluminium hydroxide (e.g., Davis et al. supra, Brazolot-Millan et al., Proc. NatL Acad. Sci., USA, 1998, 95(26), 15553-8).

Other illustrative oligonucleotides for use in compositions of the present invention will often contain two or more dinucleotide CpG motifs separated by at least three, more preferably at least six or more nucleotides. The oligonucleotides of the present invention are typically deoxynucleotides. In one embodiment the internucleotide in the oligonucleotide is phosphorodithioate, or more preferably a phosphorothioate bond, although phosphodiester and other internucleotide bonds are within the scope of the invention including oligonucleotides with mixed internucleotide linkages. Methods for producing phosphorothioate oligonucleotides or phosphorodithioate are described in U.S. Pat. Nos. 5,666,153, 5,278,302 and WO95/26204.

Other examples of oligonucleotides have sequences that are disclosed in the following publications; for certain herein disclosed embodiments the sequences preferably contain phosphorothioate modified internucleotide linkages:

CPG 7909: Cooper et al., “CPG 7909 adjuvant improves hepatitis B virus vaccine seroprotection in antiretroviral-treated HIV-infected adults.” AIDS, 2005 Sep. 23; 19(14):1473-9.

CpG 10101: Bayes et al., “Gateways to clinical trials.” Methods Find. Exp. Clin. Pharmacol. 2005 April; 27(3):193-219.

Vollmer J., “Progress in drug development of immunostimula-tory CpG oligodeoxynucleotide ligands for TLR9.” Expert Opinion on Biological Therapy. 2005 May; 5(5): 673-682

Alternative CpG oligonucleotides may comprise variants of the preferred sequences described in the above-cited publications that differ in that they have inconsequential nucleotide sequence substitutions, insertions, deletions and/or additions thereto. The CpG oligonucleotides utilized in certain embodiments of the present invention may be synthesized by any method known in the art (e.g., EP 468520). Conveniently, such oligonucleotides may be synthesized utilising an automated synthesizer. The oligonucleotides are typically deoxynucleotides. In a preferred embodiment the internucleotide bond in the oligonucleotide is phosphorodithioate, or more preferably phosphorothioate bond, although phosphodiesters are also within the scope of the presently contemplated embodiments. Oligonucleotides comprising different internucleotide linkages are also contemplated, e.g., mixed phosphorothioate phophodiesters. Other internucleotide bonds which stabilize the oligonucleotide may also be used.

In certain more specific embodiments the TLR agonist is selected from lipopolysaccharide, peptidoglycan, polyl:C, CpG, 3M003, flagellin, Leishmania homolog of eukaryotic ribosomal elongation and initiation factor 4a (LeIF) and at least one hepatitis C antigen.

Still other illustrative adjuvants include imiquimod, gardiquimod and resiquimod (all available from Invivogen), and related compounds, which are known to act as TLR7/8 agonists. A compendium of adjuvants that may be useful in vaccines is provided by Vogel et al., Pharm Biotechnol 6:141 (1995), which is herein incorporated by reference.

Compositions of the invention may also employ adjuvant systems designed to induce an immune response predominantly of the Th1 type. High levels of Th1-type cytokines (e.g., IFN-y, TNF-a., IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly of the Th1-type, the level of Th1-type cytokines will increase to a greater extent than the level of Th2-type cytokines. The levels of these cytokines may be readily assessed using standard assays. For a review of the families of cytokines, see Mossman & Coffman, Ann. Rev. Immunol. 7:145-173 (1989).

Certain adjuvants for use in eliciting a predominantly Th1-type response include, for example, a combination of monophosphoryl lipid A, preferably 3-de-O-acylated monophosphoryl lipid A (3D-MPLTM), together with an aluminum salt (U.S. Pat. Nos. 4,436,727; 4,877,611; 4,866,034; and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352 (1996). Another illustrative adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins. Other illustrative formulations include more than one saponin in the adjuvant combinations of the present invention, for example combinations of at least two of the following group comprising QS21, QS7, Quil A, 0-escin, or digitonin.

In a particular embodiment, the adjuvant system includes the combination of a monophosphoryl lipid A and a saponin derivative, such as the combination of QS21 and 3D-MPLTM adjuvant, as described in WO 94/00153, or a less reactogenic composition where the QS21 is quenched with cholesterol, as described in WO 96/33739. Other formulations comprise an oil-in-water emulsion and tocopherol. Another adjuvant formulation employing QS21, 3D-MPLTM adjuvant and tocopherol in an oil-in-water emulsion is described in WO 95/17210.

In certain preferred embodiments, the adjuvant used in the present invention is a glucopyranosyl lipid A (GLA) adjuvant, as described in U.S. Patent Application Publication No. 20080131466, the disclosure of which is incorporated herein by reference in its entirety. In one embodiment, the GLA adjuvant used in the context of the present invention has the following structure:

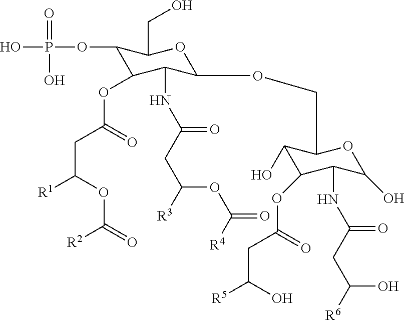

where: R¹, R³, R⁵ and R⁶ are C₁₁-C₂₀ alkyl; and R² and R⁴ are C₉-C₂₀ alkyl.

In a more specific embodiment, the GLA has the formula set forth above wherein R¹, R³, R⁵ and R⁶ are C₁₁₋₁₄ alkyl; and R² and R⁴ are C₁₂₋₁₅ alkyl.

In a more specific embodiment, the GLA has the formula set forth above wherein R¹, R³, R⁵ and R⁶ are C₁₁ alkyl; and R² and R⁴ are C₁₃ alkyl.

In a more specific embodiment, the GLA has the formula set forth above wherein R¹, R³, R⁵ and R⁶ are C₁₁ alkyl; and R² and R⁴ are C₉ alkyl.

In certain embodiments, the adjuvant is a GLA adjuvant (e.g., synthetic) having the following structure:

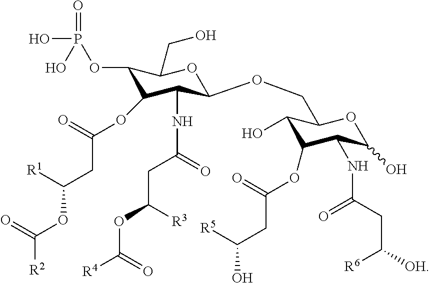

In certain embodiments of the above GLA structure, R¹, R³, R⁵ and R⁶ are C₁₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the adjuvant is a synthetic GLA adjuvant having the following structure:

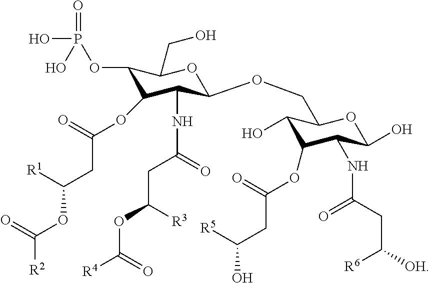

In certain embodiments of the above GLA structure, R¹, R³, R⁵ and R⁶ are C₁₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the adjuvant is a synthetic GLA adjuvant having the following structure:

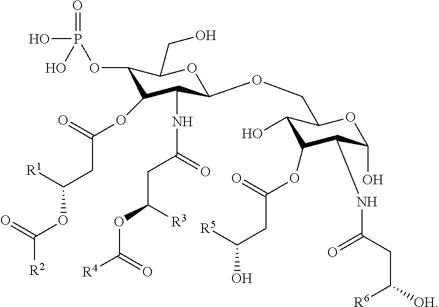

In certain embodiments of the above GLA structure, R¹, R³, R⁵ and R⁶ are C₁₁-C₂₀ alkyl; and R² and R⁴ are C₉-C₂₀ alkyl. In certain embodiments, R¹, R³, R⁵ and R⁶ are C₁₁ alkyl; and R² and R⁴ are C₉ alkyl.

In certain embodiments, the adjuvant is a synthetic GLA adjuvant having the following structure:

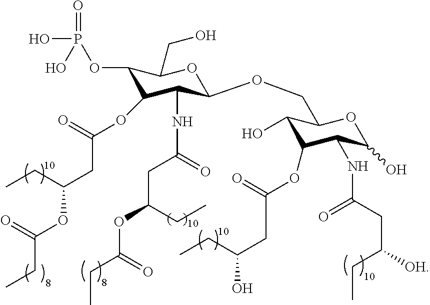

In certain embodiments, the adjuvant is a synthetic GLA adjuvant having the following structure:

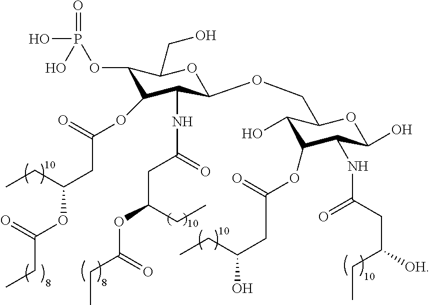

In certain embodiments, the adjuvant is a synthetic GLA adjuvant having the following structure:

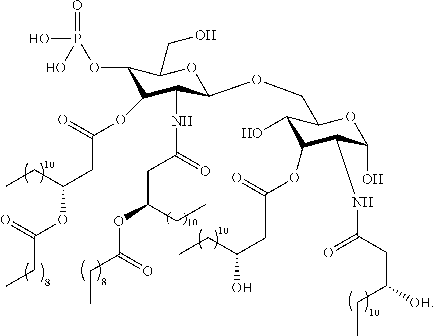

Another enhanced adjuvant system involves the combination of a CpG-containing oligonucleotide and a saponin derivative as disclosed in WO 00/09159.

Other illustrative adjuvants include Montanide ISA 720 (Seppic, France), SAF (Chiron, Calif., United States), ISCOMS (CSL), MF-59 (Chiron), the SBAS series of adjuvants (e.g., SBAS-2, AS2′, AS2,″ SBAS-4, or SBAS6, available from SmithKline Beecham, Rixensart, Belgium), Detox, RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties, and polyoxyethylene ether adjuvants such as those described in WO 99/52549A1. The vaccine and pharmaceutical compositions of the invention may be formulated using any of a variety of well known procedures. In certain embodiments, the vaccine or pharmaceutical compositions are prepared as stable emulsions (e.g., oil-in-water emulsions) or as aqueous solutions.

Compositions of the invention may also, or alternatively, comprise T cells specific for fusion polypeptide comprising immunogenic/antigenic portions or fragments of Leishmania antigens or variants thereof, described herein. Such cells may generally be prepared in vitro or ex vivo, using standard procedures. For example, T cells may be isolated from bone marrow, peripheral blood, or a fraction of bone marrow or peripheral blood of a patient. Alternatively, T cells may be derived from related or unrelated humans, non-human mammals, cell lines or cultures.

T cells may be stimulated with a fusion polypeptide comprising Leishmania polypeptides or immunogenic portions or variants thereof, polynucleotide encoding such a fusion polypeptide, and/or an antigen presenting cell (APC) that expresses such a fusion polypeptide. Such stimulation is performed under conditions and for a time sufficient to permit the generation of T cells that are specific for the polypeptide. In certain embodiments, the polypeptide or polynucleotide is present within a delivery vehicle, such as a microsphere, to facilitate the generation of specific T cells.

T cells are considered to be specific for a fusion polypeptide of the invention if the T cells specifically proliferate, secrete cytokines or kill target cells coated with the fusion polypeptide or expressing a gene encoding the fusion polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070 (1994)). Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with a polypeptide of the invention (10 Ong/ml-1001.1 g/ml, preferably 200 ng/ml-251.1 g/ml) for 3-7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-y) is indicative of T cell activation (see Coligan et al., Current Protocols in Immunology, vol. 1 (1998)). T cells that have been activated in response to a polypeptide, polynucleotide or polypeptide-expressing APC may be CD4+ and/or CD8+. Protein-specific T cells may be expanded using standard techniques. Within preferred embodiments, the T cells are derived from a patient, a related donor or an unrelated donor, and are administered to the patient following stimulation and expansion.

In the compositions of the invention, formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, intradermal, subcutaneous and intramuscular administration and formulation.

In certain applications, the compositions disclosed herein may be delivered via oral administration to a subject. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain circumstances it will be desirable to deliver the compositions disclosed herein parenterally, intravenously, intramuscularly, or even intraperitoneally as described, for example, in U.S. Pat. No. 5,543,158; U.S. Pat. No. 5,641,515 and U.S. Pat. No. 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion (see, e.g., Remington's Pharmaceutical Sciences, 15th Edition, pp. 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with the various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxy groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective for treatment of leishmaniasis. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known to one of ordinary skill in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood to one of ordinary skill in the art. Typically, such compositions are prepared as injectables, either as liquid solutions or suspensions; solid forms suitable for solution in, or suspension in, liquid prior to injection can also be prepared. The preparation can also be emulsified.

In certain embodiments, the compositions of the present invention may be delivered by intranasal sprays, inhalation, and/or other aerosol delivery vehicles. Methods for delivering genes, polynucleotides, and peptide compositions directly to the lungs via nasal aerosol sprays has been described e.g., in U.S. Pat. No. 5,756,353 and U.S. Pat. No. 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

In certain embodiments, the delivery may occur by use of liposomes, nanocapsules, microparticles, microspheres, lipid particles, vesicles, and the like, for the introduction of compositions comprising a fusion polypeptide as describe herein into suitable host cells. In particular, the compositions of the present invention may be formulated for delivery either encapsulated in a lipid particle, a liposome, a vesicle, a nanosphere, a nanoparticle or the like. The formulation and use of such delivery vehicles can be carried out using known and conventional techniques.

A pharmaceutical or immunogenic composition may, alternatively, contain an immunostimulant and a DNA molecule encoding one or more of the polypeptides or fusion polypeptides as described above, such that a desired polypeptide is generated in situ. In such compositions, the DNA encoding the fusion protein may be present within any of a variety of delivery systems known to those of ordinary skill in the art, including nucleic acid expression systems, bacteria and viral expression systems. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface. In a particular embodiment, the DNA may be introduced using a viral expression system (e.g., vaccinia or other pox virus, retrovirus, or adenovirus), which may involve the use of a non-pathogenic (defective), replication competent virus. Techniques for incorporating DNA into such expression systems are well known to those of ordinary skill in the art. The DNA may also be “naked,” as described, for example, in Ulmer et al., Science 259:1745-1749 (1993) and reviewed by Cohen, Science 259:1691-1692 (1993). The uptake of naked DNA may be increased by coating the DNA onto biodegradable beads, which are efficiently transported into the cells.

The pharmaceutical compositions and vaccines of the invention may be used, in certain embodiments, to induce protective immunity against Leishmania species such as L. donovani, L. major and/or L. infantum in a patient, such as a human or a dog, to prevent leishmaniasis or diminish its severity. The compositions and vaccines may also be used to stimulate an immune response, which may be cellular and/or humoral, in a patient, for treating an individual already infected. In one embodiment, for Leishmania-infected patients, the immune responses generated include a preferential Th1 immune response (i.e., a response characterized by the production of the cytokines interleukin-1, interleukin-2, interleukin-12 and/or interferon-y, as well as tumor necrosis factor-a). In another embodiment, for uninfected patients, the immune response involves production of interleukin-12 and/or interleukin-2, or the stimulation of gamma delta T-cells. In either category of patient, the response stimulated may include IL-12 production. Such responses may also be elicited in biological samples of PBMC or components thereof derived from Leishmania-infected or uninfected individuals. As noted above, assays for any of the above cytokines, as well as other known cytokines, may generally be performed using methods known to those of ordinary skill in the art, such as an enzyme-linked immunosorbent assay (ELISA).

Appropriate doses and methods of fusion polypeptide administration for these purposes can be readily determined by a skilled artisan using available knowledge in the art and/or routine techniques. Routes and frequency of administration, as well as dosage, for the above aspects of the present invention may vary from individual to individual and may parallel those currently being used in immunization against other infections, including protozoan, viral and bacterial infections. For example, in one embodiment, between 1 and 12 doses of composition having a fusion polypeptide, which comprises Leishmania polypeptides or immunogenic/antigenic portions, fragments or variants thereof, are administered over a 1 year period. Booster vaccinations may be given periodically thereafter as needed or desired. Of course, alternate protocols may be appropriate for individual patients. In a particular embodiment, a suitable dose is an amount of fusion polypeptide or DNA encoding such a peptide that, when administered as described above, is capable of eliciting an immune response in an immunized patient sufficient to protect the patient from leishmaniasis caused by Leishmania species such as L. donovani, L. major and/or L. infantum for at least 1-2 years. In general, the amount of fusion polypeptide present in a dose (or produced in situ by the DNA in a dose) ranges from about 100 ng to about 1 mg per kg of host, typically from about 101.1 g to about 100 ug. Suitable dose sizes will vary with the size of the patient, but will typically range from about 0.1 mL to about 5 mL.

### Diagnostic Compositions, Methods and Kits

In another aspect, this invention provides compounds and methods for detecting leishmaniasis in individuals and in blood supplies. In particular embodiments, the individual is a mammal. In more particular embodiments, the mammal is a human or canine.

For example, the fusion polypeptides and polynucleotides of the present invention can be used as effective diagnostic reagents for detecting and/or monitoring Leishmania infection in a patient. For example, the compositions, fusion polypeptides, and polynucleotides of the invention may be used in in vitro and in vivo assays for detecting humoral antibodies or cell-mediated immunity against Leishmania for diagnosis of infection, monitoring of disease progression or test-of-cure evaluation. In particular embodiments, the fusion polypeptides and polynucleotides are useful diagnostic reagents for serodiagnosis and whole blood assay in patients having leishmaniasis caused by Leishmania species such as L. donovani, L. major and/or L. infantum.

In one aspect, the diagnostic methods and kits preferably employ a polypeptide or fusion polypeptide as described herein, repeats of polypeptide fragments, or multimeric polypeptide fragments, including antigenic/immunogenic fragments. In another more specific aspect, fusion polypeptides of the present invention may comprise two or more Leishmania antigen fragments. In a more particular embodiment, an illustrative fusion polypeptide comprises the amino acid sequence set forth in SEQ ID NO: 2, 4, 6, 8, 10, 12, 14, 16, 18, 20, 41, 43, 45, 47, or 49. In another embodiment, the diagnostic methods and kits preferably employ a fusion polypeptide comprising at least 1, at least 2, at least 3, or at least 4 immunogenic/antigenic portions or fragments of Leishmania polypeptides, variants or the like, optionally in combination with one or more other Leishmania antigens or non-Leishmania sequences, as described herein or obtainable in the art.

The antigens or polypeptides may be used in essentially any assay format desired, e.g., as individual antigens assayed separately, as multiple antigens assays simultaneously (e.g., a fusion polypeptide), as antigens immobilized on a solid support such as an array, or the like.

In one embodiment, there are provided diagnostic kits for detecting Leishmania infection in a biological sample, comprising (a) a polypeptide or a fusion polypeptide described herein or variants thereof as described herein, and (b) a detection reagent.

In another embodiment, there are provided diagnostic kits for detecting Leishmania infection in a biological sample, comprising (a) antibodies or antigen binding fragments thereof that are specific for a polypeptide or a fusion polypeptides described herein or variants thereof as described herein, and (b) a detection reagent.

In another embodiment, methods are provided for detecting the presence of Leishmania infection in a biological sample, comprising (a) contacting a biological sample with a polypeptide or a fusion polypeptide described herein or variants thereof described herein; and (b) detecting in the biological sample the presence of antibodies that bind to the fusion polypeptide.

In another embodiment, methods are provided for detecting the presence of Leishmania infection in a biological sample, comprising (a) contacting a biological sample with at least 2 monoclonal antibodies that bind to a polypeptide or a polypeptide described herein or variants thereof described herein; and (b) detecting in the biological sample the presence of Leishmania proteins that bind to the monoclonal antibody.

One of ordinary skill in the art would recognize that the methods and kits described herein may be used to detect all types of leishmaniasis, depending on the particular combination of immunogenic portions of Leishmania antigens present in the fusion polypeptide.

There are a variety of assay formats known to those of ordinary skill in the art for using a fusion polypeptide to detect antibodies in a sample. See, e.g., Harlow and Lane, Antibodies. A Laboratory Manual, Cold Spring Harbor Laboratory Press, 1988, which is incorporated herein by reference. In one embodiment, the assay involves the use of fusion polypeptide immobilized on a solid support to bind to and remove the antibody from the sample. The bound antibody may then be detected using a detection reagent that binds to the antibody/peptide complex and contains a detectable reporter group. Suitable detection reagents are well known and include, for example, antibodies that bind to the antibody/polypeptide complex and free polypeptide labeled with a reporter group (e.g., in a semi-competitive assay). Suitable reporter groups are also well known and include, for example, fluorescent labels, enzyme labels, radioisotopes, chemiluminescent labels, electrochemiluminescent labels, bioluminescent labels, polymers, polymer particles, metal particles, haptens, and dyes. Alternatively, a competitive assay may be utilized, in which an antibody that binds to a fusion polypeptide of the present invention labeled with a reporter group and allowed to bind to the immobilized fusion polypeptide after incubation of the fusion polypeptide with the sample. The extent to which components of the sample inhibit the binding of the labeled antibody to the fusion polypeptide is indicative of the reactivity of the sample with the immobilized fusion polypeptide.

The solid support may be any material known to those of ordinary skill in the art to which the fusion polypeptide may be attached. For example, the support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681.

The fusion polypeptide may be bound to the solid support using a variety of techniques known to those in the art, which are amply described in the patent and scientific literature. In the context of the present invention, the term “bound” refers to both non-covalent association, such as adsorption, and covalent attachment (which may be a direct linkage between the antigen and functional groups on the support or may be a linkage by way of a cross-linking agent). Binding by adsorption to a well in a microtiter plate or to a membrane is preferred. In such cases, adsorption may be achieved by contacting the polypeptide, in a suitable buffer, with the solid support for a suitable amount of time. The contact time varies with temperature, but is typically between about 1 hour and 1 day. In general, contacting a well of a plastic microtiter plate (such as polystyrene or polyvinylchloride) with an amount of fusion polypeptide ranging from about 10 ng to about 1 pg, and preferably about 100 ng, is sufficient to bind an adequate amount of antigen. Nitrocellulose will bind approximately 100 pg of protein per 3 cm.

Covalent attachment of fusion polypeptide to a solid support may generally be achieved by first reacting the support with a bifunctional reagent that will react with both the support and a functional group, such as a hydroxyl or amino group, on the fusion polypeptide. For example, the fusion polypeptide may be bound to a support having an appropriate polymer coating using benzoquinone or by condensation of an aldehyde group on the support with an amine and an active hydrogen on the polypeptide (see, e.g., Pierce Immunotechnology Catalog and Handbook (1991) at Al2-A13).

In certain embodiments, the assay is an enzyme linked immunosorbent assay (ELISA). This assay may be performed by first contacting a fusion polypeptide of the present invention that has been immobilized on a solid support, commonly the well of a microtiter plate, with the sample, such that antibodies to the Leishmania antigens of the fusion polypeptide within the sample are allowed to bind to the immobilized fusion polypeptide. Unbound sample is then removed from the immobilized fusion polypeptide and a detection reagent capable of binding to the immobilized antibody-polypeptide complex is added. The amount of detection reagent that remains bound to the solid support is then determined using a method appropriate for the specific detection reagent.

Once the fusion polypeptide is immobilized on the support, the remaining protein binding sites on the support are typically blocked. Any suitable blocking agent known to those of ordinary skill in the art, such as bovine serum albumin (BSA) or Tween 2OTM (Sigma Chemical Co., St. Louis, Mo.) may be employed. The immobilized polypeptide is then incubated with the sample, and antibody (if present in the sample) is allowed to bind to the antigen. The sample may be diluted with a suitable diluent, such as phosphate-buffered saline (PBS) prior to incubation. In general, an appropriate contact time (i.e., incubation time) is that period of time that is sufficient to permit detection of the presence of antibody within a Leishmania-infected sample. Preferably, the contact time is sufficient to achieve a level of binding that is at least 95% of that achieved at equilibrium between bound and unbound antibody. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween 2OTM. Detection reagent may then be added to the solid support. An appropriate detection reagent is any compound that binds to the immobilized antibody-polypeptide complex and that can be detected by any of a variety of means known to those in the art. Preferably, the detection reagent contains a binding agent (such as, for example, Protein A, Protein G, immunoglobulin, lectin or free antigen) conjugated to a reporter group. Preferred reporter groups include enzymes (such as horseradish peroxidase), substrates, cofactors, inhibitors, dyes, radionuclides, luminescent groups, fluorescent groups, colloidal gold and biotin. The conjugation of binding agent to reporter group may be achieved using standard methods known to those of ordinary skill in the art. Common binding agents may also be purchased conjugated to a variety of reporter groups from many sources (e.g., Zymed Laboratories, San Francisco, Calif. and Pierce, Rockford, Ill.).

The detection reagent is then incubated with the immobilized antibody polypeptide complex for an amount of time sufficient to detect the bound antibody. An appropriate amount of time may generally be determined from the manufacturer's instructions or by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of anti-Leishmania antibodies in the sample, the signal detected from the reporter group that remains bound to the solid support is generally compared to a signal that corresponds to a predetermined cut-off value. In one embodiment, the cut-off value is preferably the average mean signal obtained when the immobilized polypeptide is incubated with samples from an uninfected patient. In general, a sample generating a signal that is three standard deviations above the predetermined cut-off value is considered positive (i.e., reactive with the polypeptide). In an alternate embodiment, the cut-off value is determined using a Receiver Operator Curve, according to the method of Sackett et al., Clinical Epidemiology: A Basic Science for Clinical Medicine, p. 106-7 (Little Brown and Co., 1985). Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper lefthand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate.

In other embodiments, an assay is performed in a flow-through assay format, wherein the antigen is immobilized on a membrane such as nitrocellulose. In the flow-through test, antibodies within the sample bind to the immobilized polypeptide as the sample passes through the membrane. A detection reagent (e.g., protein A-colloidal gold) then binds to the antibody-polypeptide complex as the solution containing the detection reagent flows through the membrane. The detection of bound detection reagent may then be performed as described above.

In other embodiments, an assay if performed in a strip test format, also known as a lateral flow format. Here, one end of the membrane to which polypeptide is bound is immersed in a solution containing the sample. The sample migrates along the membrane through a region containing detection reagent and to the area of immobilized fusion polypeptide. Concentration of detection reagent at the fusion polypeptide indicates the presence of Leishmania antibodies in the sample. Typically, the concentration of detection reagent at that site generates a pattern, such as a line, that can be read visually. The absence of such a pattern indicates a negative result. In general, the amount of fusion polypeptide immobilized on the membrane is selected to generate a visually discernible pattern when the biological sample contains a level of antibodies that would be sufficient to generate a positive signal in an ELISA, as discussed above. Preferably, the amount of fusion polypeptide immobilized on the membrane ranges from about 25 ng to about 1 fag, and more preferably from about 50 ng to about 500 ng. Such tests can typically be performed with a very small amount (e.g., one drop) of patient serum or blood. Lateral flow tests can operate as either competitive or sandwich assays.

In still other embodiments, a fusion polypeptide of the invention is adapted for use in a dual path platform (DPP) assay. Such assays are described, for example, in U.S. Pat. No. 7,189,522, the contents of which are incorporated herein by reference.

Of course, numerous other assay protocols exist that are suitable for use with the fusion polypeptides of the present invention. It will be understood that the above descriptions are intended to be exemplary only.

The assays discussed above may be used, in certain aspects of the invention, to specifically detect visceral leishmaniasis. In this aspect, antibodies in the sample may be detected using a fusion polypeptide of the present invention, e.g., comprising an amino acid sequence of antigenic/immunogenic fragments or epitopes of Leishmania antigens. Preferably, the Leishmania antigens are immobilized by adsorption to a solid support such as a well of a microtiter plate or a membrane, as described above, in roughly similar amounts such that the total amount of fusion polypeptide in contact with the support ranges from about 10 ng to about 100 pg. The remainder of the steps in the assay may generally be performed as described above. It will be readily apparent to those of ordinary skill in the art that, by combining polypeptides described herein with other polypeptides that can detect cutaneous and mucosal leishmaniasis, the polypeptides disclosed herein may be used in methods that detect all types of leishmaniasis.

In another aspect of this invention, immobilized fusion polypeptides may be used to purify antibodies that bind thereto. Such antibodies may be prepared by any of a variety of techniques known to those of ordinary skill in the art. See, e.g., Harlow and Land, Antibodies. A Laboratory Manual, Cold Spring Harbor Laboratory Press, 1988. In one such technique, an immunogen comprising a fusion polypeptide of the present invention is initially injected into any of a wide variety of mammals (e.g., mice, rats, rabbits, sheep and goats). In this step, the polypeptide may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for the antigenic fusion polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and tested for binding activity against the polypeptide. Hybridomas having high reactivity and specificity are preferred.

Monoclonal antibodies may be isolated from the supernatants of growing hybridoma colonies. In this process, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. One or more polypeptides may be used in the purification process in, for example, an affinity chromatography step.

Monospecific antibodies that bind to a fusion polypeptide comprising two or more immunogenic portions of Leishmania antigens may be used, for example, to detect Leishmania infection in a biological sample using one of a variety of immunoassays, which may be direct or competitive. Briefly, in one direct assay format, a monospecific antibody may be immobilized on a solid support (as described above) and contacted with the sample to be tested. After removal of the unbound sample, a second monospecific antibody, which has been labeled with a reporter group, may be added and used to detect bound antigen. In an exemplary competitive assay, the sample may be combined with the monoclonal or polyclonal antibody, which has been labeled with a suitable reporter group. The mixture of sample and antibody may then be combined with polypeptide antigen immobilized on a suitable solid support. Antibody that has not bound to an antigen in the sample is allowed to bind to the immobilized antigen and the remainder of the sample and antibody is removed. The level of antibody bound to the solid support is inversely related to the level of antigen in the sample. Thus, a lower level of antibody bound to the solid support indicates the presence of Leishmania in the sample. Other formats for using monospecific antibodies to detect Leishmania in a sample will be apparent to those of ordinary skill in the art, and the above formats are provided solely for exemplary purposes.

As used in this specification and the appended claims, the singular forms “a”, “an” and “the” include plural referents unless the content clearly dictates otherwise. Thus, for example, reference to “a polypeptide” optionally includes two or more polypeptides, and the like.

It is understood that aspect and embodiments of the invention described herein include “comprising,” “consisting,” and “consisting essentially of” aspects and embodiments.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Aspects of the embodiments can be modified, if necessary to employ concepts of the various patents, applications and publications to provide yet further embodiments.

### EXAMPLES

### Example 1

### Construction of Fusion Polypeptides of the Invention

821X Fusion Polypeptide.

The fusion polypeptide referred to as 821X was generated by the tandem linkage of an open reading frame of polynucleotides encoding a methionine initiation codon (ATG) added to the 5′ end of a fragment of the carboxy-terminus of the putative mitochondrial HSP70 (8E or 8) polynucleotide, the open reading frame of polynucleotides encoding the p21 antigen polypeptide, and the open reading frame of polynucleotides encoding a putative carboxypeptidase polypeptide. 821X has a 2,541 polynucleotide sequence as set forth in SEQ ID: 1 which comprises polynucleotides 1 to 459 which encodes amino acids 509 to 660 of the carboxy-terminus of the putative mitochondrial HSP70 (8E or 8) polypeptide from L. infantum, polynucleotides 460 to 1032 which encodes amino acids 1 to 191 of the of the p21 antigen polypeptide (p21 or 21) of Leishmania infantum, and polynucleotides 1033 to 2541 which encodes amino acids 1 to 503 of the putative carboxypeptidase (CxP or X) polypeptide of L donovani. 821X has a polypeptide sequence set forth in SEQ ID NO: 2 which comprises amino acids 509 to 660 of the carboxy-terminus of the putative mitochondrial HSP70 (8E or 8) polypeptide from L infantum, amino acids 1 to 191 of the p21 antigen protein from Leishmania donovani, and amino acids 1 to 503 of the putative carboxypeptidase (CxP or X) polypeptide of L donovani. The 847 amino acid fusion polypeptide with a predicted mass of 95,803 Daltons was expressed in E. coli and purified by column chromatography.

821XH Fusion Polypeptide.

The fusion polypeptide referred to as 821XH was generated by the tandem linkage of an open reading frame of polynucleotides encoding a methionine initiation codon (ATG) added to the 5′ end of a fragment of the carboxy-terminus of the putative mitochondrial HSP70 (8E or 8) polynucleotide, an open reading frame of polynucleotides encoding p21 antigen polypeptide (p21 or 21), the open reading frame of polynucleotides encoding putative carboxypeptidase polypeptide (CxP or X), and an open reading frame of polynucleotides encoding the amino terminus of the histone H2BN polypeptide (H2BN, h2Bn or H). 821XH has a 2,679 polynucleotide sequence as set forth in SEQ ID: 3 which comprises polynucleotides 1 to 459 which encodes amino acids 509 to 660 of the carboxy-terminus of the putative mitochondrial HSP70 (8E or 8) polypeptide from L infantum, polynucleotides 460 to 1032 which encodes amino acids 1 to 191 of the of the p21(p21 or 21) antigen polypeptide of Leishmania infantum, polynucleotides 1033 to 2541 which encodes amino acids 1 to 503 of the putative carboxypeptidase (CxP or X) polypeptide of L donovani, and polynucleotides 2542 to 2679 which encodes amino acids 1 to 46 of the amino terminus of the histone H2BN (H) polypeptide from L infantum. 821XH has a polypeptide sequence set forth in SEQ ID NO: 4 which comprises amino acids 509 to 660 of the carboxy-terminus of the putative mitochondrial HSP70 (8E or 8) polypeptide from L infantum, amino acids 1 to 191 of the p21 antigen polypeptide from Leishmania donovani, amino acids 1 to 503 of the putative carboxypeptidase polypeptide of L donovani and amino acids 1 to 46 of the amino terminus of the histone H2BN (H) polypeptide from L infantum. The 893 amino acid fusion polypeptide with a predicted mass of 101,016 Daltons was expressed in E. coli and purified by column chromatography.

821XA Fusion Polypeptide.

The fusion polypeptide referred to as 821XA was generated by the tandem linkage of an open reading frame of polynucleotides encoding a methionine initiation codon (ATG) added to the 5′ end of a fragment of the carboxy-terminus of the putative mitochondrial HSP70 (8E or 8) polynucleotide, an open reading frame of polynucleotides encoding p21 antigen polypeptide (p21 or 21), the open reading frame of polynucleotides encoding a putative carboxypeptidase polypeptide (CxP or C), and an open reading of polynucleotides encoding the mature A2 polypeptide (A2 or A). 821XA has a 3,183 polynucleotide sequence as set forth in SEQ ID: 5 which comprises polynucleotides 1 to 456 which encodes amino acids 509-660 of the carboxy-terminus of the putative mitochondrial HSP70 (8E or 8) gene from L infantum, polynucleotides 460 to 1032 which encodes amino acids 1 to 191 of the of the p21 antigen of Leishmania infantum, polynucleotides 1033 to 2541 which encodes amino acids 1 to 503 of the putative carboxypeptidase (CxP) of L donovani, and polynucleotides 2542 to 3183 which encodes amino acids 23 to 236 of the mature A2 polypeptide from Leishmania donovani. 821XA has a polypeptide sequence set forth in SEQ ID NO: 6 which comprises amino acids 509 to 660 of the carboxy-terminus of the putative mitochondrial HSP70 (8E or 8) polypeptide from L infantum, amino acids 1 to 191 of the p21 antigen polypeptide from Leishmania donovani, amino acids 1 to 503 of the putative carboxypeptidase polypeptide of L donovani and amino acids 23-236 of the mature A2 polypeptide from Leishmania donovani. The 1,061 amino acid fusion polypeptide with a predicted mass of 115,539 Daltons was expressed in E. coli and purified by column chromatography.

821NA Fusion Polypeptide.

The fusion polypeptide referred to as 821NA was generated by the tandem linkage of an open reading frame of polynucleotides encoding a methionine initiation codon (ATG) added to the 5′ end of a fragment of the carboxy-terminus of the putative mitochondrial HSP70 (8E or 8) polynucleotide, an open reading frame of polynucleotides encoding p21 antigen polypeptide (p21 or 21), the open reading frame of polynucleotides encoding the polypeptide nonspecific nucleoside hydrolase (NH, Nh or N), and the open reading frame of polynucleotides encoding the mature A2 polypeptide (A2 or A). 821NA has a 2,616 polynucleotide sequence as set forth in SEQ ID: 7 which comprises polynucleotides 1 to 459 which encodes amino acids 509 to 660 of the carboxy-terminus of the putative mitochondrial HSP70 (8E or 8) polypeptide from L infantum, polynucleotides 460 to 1032 which encodes amino acids 1 to 191 of the of the p21 antigen polypeptide of Leishmania infantum, polynucleotides 1033 to 1974 which encodes amino acids 1 to 314 of the NH polypeptide from Leishmania infantum or L donovani, and polynucleotides 1975 to 2616 which encodes amino acids 23 to 236 of the mature A2 polypeptide from Leishmania donovani. 821NA has a polypeptide sequence set forth in SEQ ID NO: 8 which comprises amino acids 509 to 660 of the carboxy-terminus of the putative mitochondrial HSP70 (8E or 8) polypeptide from L infantum, amino acids 1 to 191 of the p21 antigen polypeptide from Leishmania donovani, amino acids 1-314 of the NH polypeptide from Leishmania infantum/donovani, and amino acids 23-236 of the mature A2 polypeptide from Leishmania donovani. The 872 amino acid fusion polypeptide with a predicted mass of 92,582 Daltons was expressed in E. coli and purified by column chromatography.

NXH Fusion Polypeptide.

The fusion polypeptide referred to as NXH was generated by the tandem linkage of a Leishmania open reading frame of polynucleotides encoding the nonspecific nucleoside hydrolase (NH, Nh or N) polypeptide, the open reading frame of polynucleotides encoding the putative carboxypeptidase polypeptide (CxP or X), and an open reading frame of polynucleotides encoding the amino terminus of the histone H2BN (H2BN, h2Bn or H) polypeptide. NXH has a 2,589 nucleotide sequence as set forth in SEQ ID: 9 which comprises nucleotides 1 to 942 which encodes amino acids 1 to 314 of the NH polypeptide from Leishmania infantum or L donovani, nucleotides 943 to 2451 which encodes amino acids 1 to 314 of the full length NH polypeptide from Leishmania infantum/donovani and polynucleotides 2452 to 2589 which encodes amino acids 1 to 46 of the amino terminus of the histone H2BN (H) polypeptide from L infantum. NXH has a polypeptide sequence set forth in SEQ ID NO: 10 which comprises amino acids 1 to 314 of the full length NH polypeptide from Leishmania infantum or L donovani, amino acids 1 to 503 of the putative carboxypeptidase polypeptide of L donovani and amino acids 1 to 46 of the amino terminus of the histone H2BN (H) polypeptide from L infantum. The 863 amino acid fusion polypeptide with a predicted mass of 96,629 Daltons was expressed in E. coli and purified by column chromatography.

TXL Fusion Polypeptide.

The fusion polypeptide referred to as TXL was generated by the tandem linkage of an open reading frame of polynucleotides encoding the Leishmania major thiol specific antioxidant polypeptide (TSA or T), an open reading frame of polynucleotides encoding the putative carboxypeptidase polypeptide (CxP or C) of L donovani, and an open reading frame of polynucleotides encoding the putative eukaryotic initiation factor 4a polypeptide (Leif or L) of Leishmania major. TXL has a 2,796 polynucleotide sequence as set forth in SEQ ID: 11 which comprises polynucleotides 1-597 which encodes amino acids 1 to 199 of TSA, polynucleotides 597-2,112 which encodes amino acids 1 to 503 of the putative carboxypeptidase (CxP) of L donovani, and polynucleotides 2,113 to 2796 which encodes amino acids 1 to 226 of the Leishmania major putative eukaryotic initiation factor 4a. TXL has a polypeptide sequence set forth in SEQ ID NO: 12 which comprises amino acids 1 to 199 of the TSA protein from Leishmania major, amino acids 1 to 503 of the putative CxP of L donovani, and amino acids 1 to 226 of the Leif protein from Leishmania major. The 932 amino acid fusion polypeptide with a predicted mass of 105,134 Daltons was expressed in E. coli and purified by column chromatography.

8XHA Fusion Polypeptide.

The fusion polypeptide referred to as 8XHA was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the full length putative carboxypeptidase (CxP or X) polypeptide of L donovani, an open reading frame of polynucleotides encoding the amino terminus of the histone H2BN polypeptide (H2BN, h2Bn, or H), and an open reading of polynucleotides encoding the mature A2 polypeptide (A2 or A). 8XHA has a 2,766 polynucleotide sequence as set forth in SEQ ID: 13 which encodes amino acids 509 to 660 of the carboxy-terminus of the putative mitochondrial HSP70 (8E or 8) polypeptide from L infantum, polynucleotides 460 to 1974 which encodes amino acids 1 to 503 of the putative carboxypeptidase (CxP) polypeptide of L donovani, and polynucleotides 1975 to 2124 which encodes amino acids 1 to 46 of the amino terminus of the histone H2BN (H) polypeptide from L infantum, and polynucleotides 2125 to 2766 which encode amino acids 23 to 236 of the mature A2 polypeptide from L donovani. 8XHA has a polypeptide sequence set forth in SEQ ID NO: 14 which comprises amino acids 509 to 660 of the carboxy-terminal fragment of the putative mitochondrial HSP70 polypeptide (8e or 8) from L infantum or donovani, amino acids lto 503 of the full length putative carboxypeptidase polypeptide from L donovani (X), amino acids 1 to 46 of the of the amino terminus H2B polypeptide for L infantum (H) and amino acids 23-236 of the mature A2 polypeptide (A) from L donovani. The 922 amino acid fusion polypeptide with a predicted mass of 99,967 Daltons was expressed in E. coli and purified by column chromatography.

8NHA Fusion Polypeptide.

The fusion polypeptide referred to as 8NHA was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the full length nonspecific nucleoside hydrolase (NH, Nh or N) polypeptide, an open reading frame of polynucleotides encoding a fragment of the amino terminus of the histone H2BN polypeptide (H2BN, h2Bn, or H), and an open reading of polynucleotides encoding the mature A2 polypeptide (A2 or A). 8NHA has a 2,199 polynucleotide sequence as set forth in SEQ ID: 15 which comprises polynucleotides 1 to 459 which encodes amino acids 509 to 660 of the carboxy-terminus of the putative mitochondrial HSP70 (8E or 8) polypeptide from L infantum, polynucleotide 460 to 1407 which encodes amino acids 1 to 314 of the full length nonspecific nucleoside hydrolase polypeptide from L donovani or L infantum, polynucleotides 1408 to 1557 which encodes amino acids 1 to 46 of the amino terminus of the histone H2BN (H) polypeptide from L infantum, and polynucleotides 1558 to 2199 which encode amino acids 23 to 236 of the mature A2 polypeptide from L donovani. 8NHA has a polypeptide sequence set forth in SEQ ID NO: 16 which comprises amino acids 509 to 660 of the carboxy-terminal fragment of the putative mitochondrial HSP70 polypeptide (8e or 8), from L infantum or donovani, amino acids 1 to 314 of the full length nonspecific nucleoside hydrolase polypeptide (N) from L donovani or L infantum, amino acids 1 to 46 of the H2B polypeptide from L infantum (H) and amino acids 23-236 of the mature A2 polypeptide (A) from L donovani. The 922 amino acid fusion polypeptide with a predicted mass of 99,967 Daltons was expressed in E. coli and purified by column chromatography.

8CHA Fusion Polypeptide.

The fusion polypeptide referred to as 8CHA was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the carboxy-terminal fragment of the cysteine proteinase B polypeptide (CpB, CPB or C), an open reading frame of polynucleotides encoding a fragment of the amino terminus of the histone H2BN polypeptide (H2BN, h2Bn, or H), and an open reading of polynucleotides encoding the mature A2 polypeptide (A2 or A). 8CHA has a 2,127 polynucleotide sequence as set forth in SEQ ID: 17 which comprises polynucleotides 1 to 459 which encodes amino acids 509 to 660 of the carboxy-terminus of the putative mitochondrial HSP70 (8E or 8) polypeptide from L infantum, polynucleotide 460 to 1335 which encodes amino acids 154 to 443 of the carboxy-terminal fragment of the cysteine proteinase B polypeptide (B), polynucleotides 1336 to 1485 which encodes amino acids 1 to 46 of the amino terminus of the histone H2BN (H) polypeptide from L infantum, and polynucleotides 1486 to 2127 which encode amino acids 23 to 236 of the mature A2 polypeptide (A) from L donovani. 8CHA has a polypeptide sequence set forth in SEQ ID NO: 18 which comprises amino acids 509 to 660 of the carboxy-terminal fragment of the putative mitochondrial HSP70 polypeptide (8e or 8) from L infantum or donovani, amino acids 154 to 143 of the carboxy-terminal fragment of the CpB polypeptide of L infantum (C), amino acids 1 to 46 of the H2B polypeptide from L infantum (H) and amino acids 23-236 of the mature A2 polypeptide (A) from L donovani. The 709 amino acid fusion polypeptide with a predicted mass of 73, 633 Daltons was expressed in E. coli and purified by column chromatography

8NCA Fusion Polypeptide.

The fusion polypeptide referred to as 8NCA was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the full length nonspecific nucleoside hydrolase polypeptide (NH, Nh or N), an open reading frame of polynucleotides encoding the carboxy-terminal fragment of the cysteine proteinase B polypeptide (CpB, CPB or C), and an open reading of polynucleotides encoding the mature A2 polypeptide (A2 or A). 8NCA has a 2,931 polynucleotide sequence as set forth in SEQ ID: 19 which comprises polynucleotides 1 to 459 which encodes amino acids 509 to 660 of the carboxy-terminus of the putative mitochondrial HSP70 (8E or 8) polypeptide from L infantum, polynucleotides 460 to 1407 which encodes amino acids 1 to 314 of the full length nonspecific nucleoside hydrolase polypeptide from L infantum or L donovani, polynucleotides 1408 to 283 which encodes amino acids 154 to 443 of the carboxy terminal fragment of the cysteine proteinase B polypeptide from L infantum, and polynucleotides 2284 to 2931 which encode amino acids 23 to 236 of the mature A2 polypeptide from L donovani. 8NCA has a polypeptide sequence set forth in SEQ ID NO: 20 which comprises amino acids 509 to 660 of the carboxy-terminal fragment of the putative mitochondrial HSP70 polypeptide (8e or 8) from L infantum or donovani, amino acids 1 to 314 of the NH polypeptide from L infantum or L donovani (N), amino acids 154 to 143 of the carboxy-terminal fragment of the CpB polypeptide of L infantum (C), and amino acids 23-236 of the mature A2 (A) polypeptide from L donovani. The 977 amino acid fusion polypeptide with a predicted mass of 102,641 Daltons was expressed in E. coli and purified by column chromatography.

8NC Fusion Polypeptide.

The fusion polypeptide referred to as 8NCA was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the full length nonspecific nucleoside hydrolase polypeptide (NH, Nh or N), and an open reading frame of polynucleotides encoding the carboxy-terminal fragment of the cysteine proteinase B polypeptide (CpB, CPB or C). 8NC has a 2,271 polynucleotide sequence as set forth in SEQ ID: 40 which comprises polynucleotides 1 to 459 which encodes amino acids 509 to 660 of the carboxy-terminus of the putative mitochondrial HSP70 (8E or 8) polypeptide from L infantum, and polynucleotides 460 to 1401 which encodes amino acids 1 to 314 of the full length nonspecific nucleoside hydrolase polypeptide from L infantum or L donovani, and polynucleotides 1402 to 2271 which encodes amino acids 154 to 443 of the carboxy terminal fragment of the cysteine proteinase B polypeptide from L infantum. 8NC has a polypeptide sequence set forth in SEQ ID NO: 41 which comprises amino acids 509 to 660 of the carboxy-terminal fragment of the putative mitochondrial HSP70 polypeptide (8e or 8) from L infantum or donovani, amino acids 1 to 314 of the NH polypeptide from L infantum or L donovani (N), and amino acids 154 to 143 of the carboxy-terminal fragment of the CpB polypeptide of L infantum (C). The 757 amino acid fusion polypeptide with a predicted mass of 82330 Daltons was expressed in E. coli and purified by column chromatography.

8NCH Fusion Polypeptide.

The fusion polypeptide referred to as 8NCH was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the full length nonspecific nucleoside hydrolase polypeptide (NH, Nh or N), an open reading frame of polynucleotides encoding the carboxy-terminal fragment of the cysteine proteinase B polypeptide (CpB, CPB or C), and an open reading of polynucleotides encoding the amino terminus of the histone H2BN polypeptide (H2BN, h2Bn or H). 8NCH has a 2,604 polynucleotide sequence as set forth in SEQ ID: 42 which comprises polynucleotides 1 to 459 which encodes amino acids 509 to 660 of the carboxy-terminus of the putative mitochondrial HSP70 (8E or 8) polypeptide from L infantum, polynucleotides 460 to 1401 which encodes amino acids 1 to 314 of the full length nonspecific nucleoside hydrolase polypeptide from L infantum or L donovani, polynucleotides 1402 to 2271 which encodes amino acids 154 to 443 of the carboxy terminal fragment of the cysteine proteinase B polypeptide from L infantum, and polynucleotides 2272 to 2604 which encodes amino acids 1 to 111 of the amino terminus of the histone H2BN (H) polypeptide from L infantum. 8NCH has a polypeptide sequence set forth in SEQ ID NO: 43 which comprises amino acids 509 to 660 of the carboxy-terminal fragment of the putative mitochondrial HSP70 polypeptide (8e or 8) from L infantum or donovani, amino acids 1 to 314 of the NH polypeptide from L infantum or L donovani (N), amino acids 154 to 143 of the carboxy-terminal fragment of the CpB polypeptide of L infantum (C), and amino acids 1 to 111 of the amino terminus of the histone H2BN (H) polypeptide from L infantum. The 868 amino acid fusion polypeptide with a predicted mass of 94,471 Daltons was expressed in E. coli and purified by column chromatography.

8MCH Fusion Polypeptide.

The fusion polypeptide referred to as 8MCH was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding Malate Dehydrogenase (MDH or M), an open reading frame of polynucleotides encoding the carboxy-terminal fragment of the cysteine proteinase B polypeptide (CpB, CPB or C), and an open reading of polynucleotides encoding the amino terminus of the histone H2BN polypeptide (H2BN, h2Bn or H). 8MCH has a 2,629 polynucleotide sequence as set forth in SEQ ID: 44 which comprises polynucleotides 1 to 459 which encodes amino acids 509 to 660 of the carboxy-terminus of the putative mitochondrial HSP70 (8E or 8) polypeptide from L infantum, polynucleotides 460 to 1425 which encodes amino acids 1 to 322 of the Malate Dehydrogenase polypeptide from L infantum, polynucleotides 1426 to 2295 which encodes amino acids 154 to 443 of the carboxy terminal fragment of the cysteine proteinase B polypeptide from L infantum, and polynucleotides 2296 to 2629 which encodes amino acids 1 to 46 of the amino terminus of the histone H2BN (H) polypeptide from L infantum. 8MCH has a polypeptide sequence set forth in SEQ ID NO: 45 which comprises amino acids 509 to 660 of the carboxy-terminal fragment of the putative mitochondrial HSP70 polypeptide (8e or 8) from L infantum or donovani, amino acids 1 to 322 of the MDH polypeptide from L infantum (M), amino acids 154 to 143 of the carboxy-terminal fragment of the CpB polypeptide of L infantum (C), and amino acids 1 to 111 of the amino terminus of the histone H2BN (H) polypeptide from L infantum. The 876 amino acid fusion polypeptide with a predicted mass of 93,806 Daltons was expressed in E. coli and purified by column chromatography.

8MTH Fusion Polypeptide.

The fusion polypeptide referred to as 8MTH was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding the Malate Dehydrogenase (MDH or M), an open reading frame of polynucleotides encoding Alpha Tubulin (aT or T), and an open reading of polynucleotides encoding the amino terminus of the histone H2BN polypeptide (H2BN, h2Bn or H). 8MTH has a 3,228 polynucleotide sequence as set forth in SEQ ID: 46 which comprises polynucleotides 1 to 459 which encodes amino acids 509 to 660 of the carboxy-terminus of the putative mitochondrial HSP70 (8E or 8) polypeptide from L infantum, polynucleotides 460 to 1425 which encodes amino acids 1 to 322 of the Malate Dehydrogenase polypeptide from L infantum, polynucleotides 1426 to 2894 which encodes amino acids 1 to 490 of Alpha Tubulin from L infantum, and polynucleotides 2295 to 3228 which encodes amino acids 1 to 111 of the amino terminus of the histone H2BN (H) polypeptide from L infantum. 8MCH has a polypeptide sequence set forth in SEQ ID NO: 47 which comprises amino acids 509 to 660 of the carboxy-terminal fragment of the putative mitochondrial HSP70 polypeptide (8e or 8) from L infantum or donovani, amino acids 1 to 322 of the MDH polypeptide from L infantum (M), amino acids 1 to 490 of Alpha Tubulin of L infantum (C), and amino acids 1 to 111 of the amino terminus of the histone H2BN (H) polypeptide from L infantum. The 1,076 amino acid fusion polypeptide with a predicted mass of 116,856 Daltons was expressed in E. coli and purified by column chromatography.

8TCH Fusion Polypeptide.

The fusion polypeptide referred to as 8NCH was generated by the tandem linkage of an open reading frame of polynucleotides encoding a methionine initiation codon (ATG) added to the 5′ end of a fragment of the carboxy-terminus of the putative mitochondrial HSP70 (8E or 8) polypeptide, an open reading frame of polynucleotides encoding Alpha Tubulin (aT or T), an open reading frame of polynucleotides encoding the carboxy-terminal fragment of the cysteine proteinase B polypeptide (CpB, CPB or C), and an open reading of polynucleotides encoding the amino terminus of the histone H2BN polypeptide (H2BN, h2Bn or H). 8TCH has a 3,132 polynucleotide sequence as set forth in SEQ ID: 48 which comprises polynucleotides 1 to 459 which encodes amino acids 509 to 660 of the carboxy-terminus of the putative mitochondrial HSP70 (8E or 8) polypeptide from L infantum, polynucleotides 460 to 1929 which encodes amino acids 1 to 490 of Alpha Tubulin (aT or T) from L infantum, polynucleotides 1930 to 2799 which encodes amino acids 154 to 443 of the carboxy terminal fragment of the cysteine proteinase B polypeptide from L infantum, and polynucleotides 2800 to 3132 which encodes amino acids 1 to 111 of the amino terminus of the histone H2BN (H) polypeptide from L infantum. 8TCH has a polypeptide sequence set forth in SEQ ID NO: 49 which comprises amino acids 509 to 660 of the carboxy-terminal fragment of the putative mitochondrial HSP70 polypeptide (8e or 8) from L infantum or donovani, amino acids 1 to 490 of the Alpha Tubulin (aT or T) polypeptide from L infantum (T), amino acids 154 to 143 of the carboxy-terminal fragment of the CpB polypeptide of L infantum (C), and amino acids 1 to 111 of the amino terminus of the histone H2BN (H) polypeptide from L infantum. The 1,040 amino acid fusion polypeptide with a predicted mass of 114,413 Daltons was expressed in E. coli and purified by column chromatography.

### Example 2

### Methods for Assessing Immunogenicity of Polypeptides and Fusion Polypeptides of the Invention

The Polypeptides and Fusions Polypeptides of the invention were analyzed for their ability to generate an immune response or confer protection against a visceral Leishmania donovani infection according to the methods described herein. The individual polypeptides and fusion polypeptides of the invention were for the purposes of these examples formulated within stable emulsions with the TLR4 adjuvant, GLA. Mice received a total of three immunizations three weeks apart in a prime, boost, boost strategy known in the art. Representative data for the carboxy-terminal polypeptide fragment of the putative mitochondrial HSP70 polypeptide designated 8E as the individual polypeptide or 8 in the context of a fusion polypeptide, the full length putative carboxypeptidase polypeptide designated CxP as the individual polypeptide or X in the context of a fusion polypeptide, and the fusion polypeptide 821X are presented but are not intended to be limiting of the Leishmania fusion polypeptides of the invention.

Humoral Responses:

Briefly, BALB/C mice were immunized with either 5 μg of recombinant fusion polypeptide, 5 μg of individual polypeptides of the fusions, or 5 μg of a mixture of individual polypeptides of the fusions formulated in 5 μg of an adjuvant formulation, in some examples a TLR4 stable emulsion (GLA-SE) in a total volume of 100 μl. Controls may include 5 μg of recombinant fusion polypeptide, 5 μg of individual polypeptides of the fusions, or 5 μg of a mixture of individual polypeptides of the fusions formulated within a stable emulsion without GLA (SE), saline, GLA-SE, or SE in a total volume of 100 μl. All immunizations were administered in 100 μl subcutaneously in the base of the tail (Week 0). The mice were immunized two more times (for a total of three immunizations, prime, boost, boost) at 3 week intervals with an additional 5 μg of test article in a total volume of 100 μl subcutaneously in the base of the tail (Week 3 and Week 6 Two weeks after the third immunization (Week 8) blood is obtained by insertion of a collection tube into the capillaries of the eye. Serum is prepared and tested for antigen-specific antibody responses to the fusion protein as well as the individual components of the protein by ELISA. Serum from immunized mice is titrated to find an endpoint titer (last optical density (OD) value greater than a threshold determined by sera from unimmunized mice). The antigen-specific antibody response is analyzed for total IgG against the specific antigen, and also IgG2 and IgG1 isotypes to reveal any immune bias (for example, IFNγ stimulates IgG2a/c responses while IL-4/5 stimulate IgG1 responses).

Cellular Responses:

One month (Week 10) after the final immunization, one cohort of animals was sacrificed and their spleens harvested. Briefly, duplicate wells of 2×10⁵ single cell spleen suspensions were incubated with 10 μg/ml of the appropriate polypeptide antigen to assess antigen-specific recall responses. In some experiments the animals were immunized with fusion polypeptides of the invention and replicate wells were stimulated with either fusion polypeptides, individual polypeptides of the fusion, mixtures of individual polypeptides of the fusion, or irradiated whole or prepared lysates of Leishmania parasites, or saline as a control. In some experiments the Leishmania lysates were prepared from L. donovani, L infantum, or L major. The immune response is assessed by determining the particular cell type producing cytokines by intracellular cytokine staining after 1 day (as determined by flow cytometry) and measuring secretion of cytokines into the culture supernatant after 4 days (as determined by cytokine ELISA according to the manufacturer's instructions (eBioScience)). The anticipated protective response for both cutaneous and visceral leishmaniasis (CL and VL respectively) is a Tₕₑₗₚₑᵣ1 profile, characterized by the secretion of one more cytokines including but not limited to IFNγ, TNF and IL-2 production from CD4 T cells in response to specific antigen (either the fusion polypeptide, the individual polypeptides of the fusion, or lysates of Leishmania parasites). Data is presented as percentage of cytokine positive CD4+ T cells producing the indicated cytokine or cytokines (i.e. IFNγ, IL-2, TNF, as examples). The frequency of multifunctional effector cells (T cells secreting more than one cytokine in response to recall antigen stimulation) has previously been correlated with protection against Leishmania infection.

Prophylactic Studies:

The fusion polypeptides were also evaluated for their ability to protect against visceral leishmaniasis (VL) using the BALB/c mouse model. Briefly, mice were immunized subcutaneously 3 times at 3 weeks apart (prime/boost/boost) with individual polypeptides of the fusions, mixtures of individual polypeptides of the fusion polypeptides, fusion polypeptides, irradiated Leishmania parasites, or GLA-SE or saline alone as controls. One month after the last immunization, mice were challenged via intravenous injection with up to 5×10⁶ L. donovani promastigotes. Livers were harvested one month post-challenge and parasite burdens determined by limiting dilution assay or real time PCR quantitation by methods known in the art. Reductions in parasite burden following immunization with fusion polypeptides, individual polypeptides of the fusions, mixtures of individual polypeptides of the fusions or control saline or adjuvant formulations are presented.

### Example 3

### Polyclonal Rabbit Anti-Sera to 8E or p21 Recognize Leishmania major Amastigotes

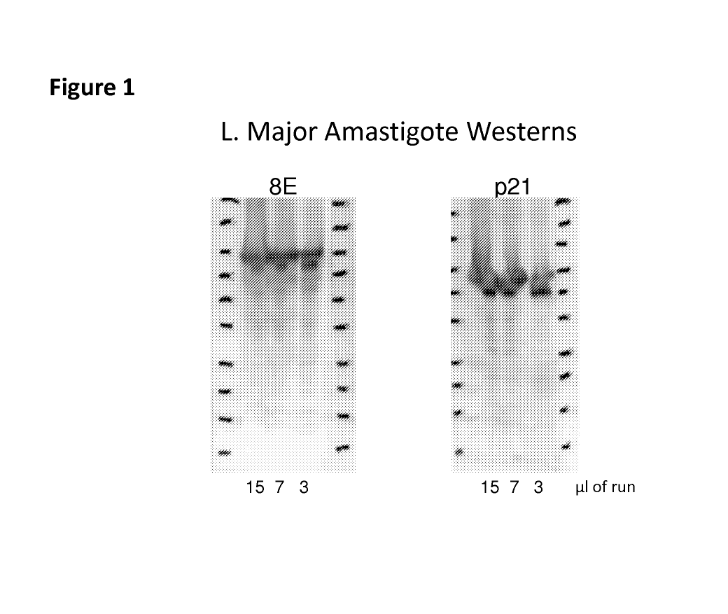

Rabbits immunized with the 8E polypeptide or the p21 polypeptide generated a polyclonal rabbit antisera that recognize L. major amastigotes. Briefly, a lysate prepared from L. major amastigotes was run on a Tris-Glycine gel and transferred onto a nitrocellulose filter. The nitrocellulose filter was blocked in 5% milk plus PBS plus 0.1% Tween 20 at room temperature for 1 hour. The filter was washed one time for 5 minutes with PBS plus 0.1% Tween 20 and incubated 2 hours at room temperature with a rabbit poly-clonal anti-sera prepared by immunizing rabbits with 8E or p21 diluted at 1/500 with PBS plus 0.1% tween 20. The membrane was then washed three time for 5 minutes each with PBS plus 0.1% tween 20. Following the last wash the membrane was incubated for one hour at room temperature with Goat anti-rabbit IgG-HRP Conjugate (BioRad), diluted at 1/7500 with PBS plus 0.1% tween 20. Subsequently the membrane was washed three times for 5 minutes with PBS plus 0.1% tween 20. Bands were visualized by Chromogenic detection using TMB membrane peroxidase substrate (KPL) according to manufacturer's directions, washed with water and allowed to dry. The data present in FIG. 1 comparing three concentrations of amastigotes (15 ul is equivalent to 3.5×10⁷ amastigotes) demonstrates that rabbit antisera raised to the mtHSP70 or p21 antigen recognize L. major amastigote lysates, the infectious stage of the parasite, indicating these antigens may be particularly useful in a vaccine.

### Example 4

### Immunogenicity of the Carboxy-Terminal Fragment of the Putative Mitochondrial HSP70 Polypeptide (Designated 8E or 8 Herein)

BALB/c mice were immunized according to the methods described in the Example and the cellular and humoral immune responses were analyzed. In addition a subset of mice were challenged with L donovani as described in Example 2 and the parasite burden was assessed.

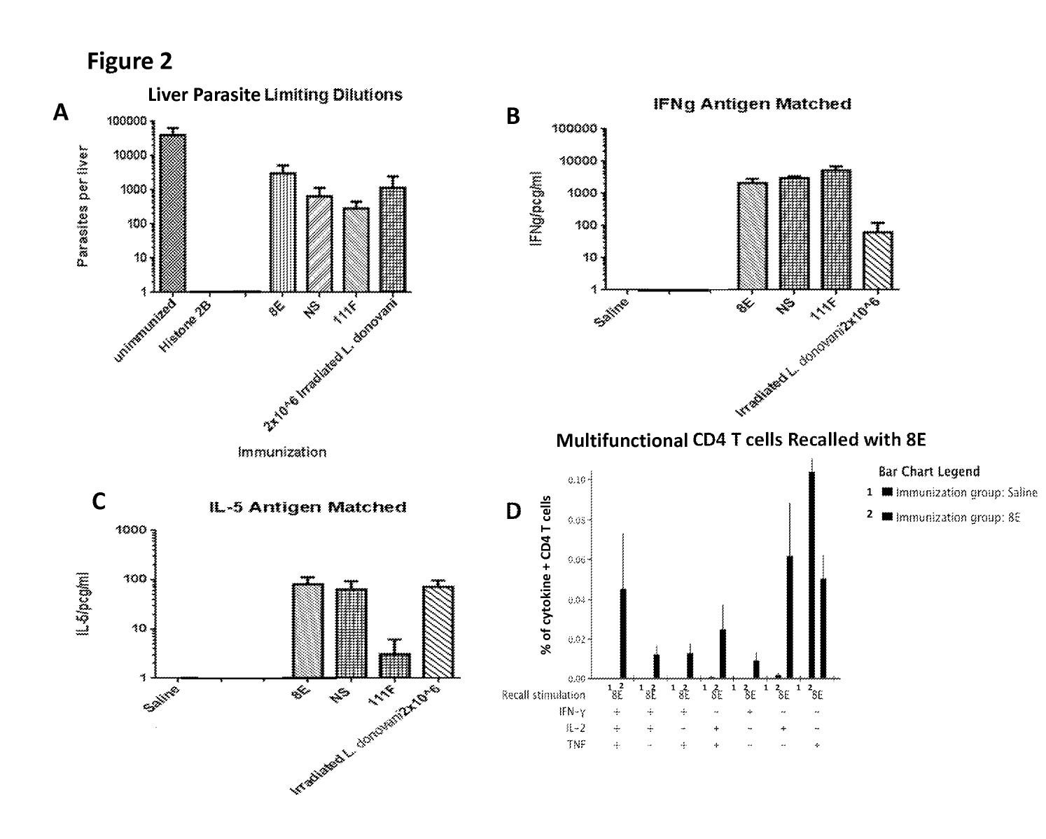

The data in FIG. 2A shows that mice immunized with 8E had a reduced L donovani burden in their livers. In addition data presented for the carboxy-terminal fragment of the putative mitochondrial HSP70 polypeptide, designated as 8E as the individual polypeptide or 8 in the context of a fusion polypeptide, demonstrates that the carboxy-terminal fragment comprising amino acids 509 to 660 of the putative mitochondrial HSP70 polypeptide demonstrates comparable protection to animal immunized with either whole irradiated L donovani parasites or NS and 111F fusion polypeptides known in the art as positive controls.

The data in FIG. 2 show spleen cells from 8E-immunized mice secreted large quantities of IFNγ (IFNg) in response to 8E restimulation (a recall response) (FIG. 2B), while little IL-5 was produced (FIG. 2C). Mice immunized with 8E possessed a CD4 T cell population that produced both IFNγ and TNF in response to restimulation with 8E alone. Flow cytometry analysis of spleen cells from mice immunized with 8E indicated that 8E specific IFNγ was produced predominantly by CD4 T cells also producing TNF (FIG. 2D).

### Example 5

### Immunogenicity of the Full Length Putative Carboxypeptidase Polypeptide (Designated CxP or X Herein)

BALB/c mice were immunized according to the methods described in the Example and the cellular and humoral immune responses were analyzed. In addition a subset of mice were challenged with L donovani as described in Example 2 and the parasite burden was assessed.

Mice immunized with CxP had a reduced L donovani burden in their livers, a mean of 3.31×10⁵ for animals immunized with CxP versus mean 4.06×10⁵ for unimmunized mice, as determined by real time PCR.

Antigen-specific (CxP) IFNγ secretion was measured in spleen cell suspension stimulated in vitro for 4 days with CxP one month following the last boost with the CxP polypeptide. Spleen cells from mice immunized with CxP demonstrated high levels of IFNγ secretion in response to CxP with a mean 4282 pg/ml versus 0 pg/ml in the unstimulated cultures. This IFNγ response was in contrast to the very low amounts of IL-5 detected, 36 pg/ml.

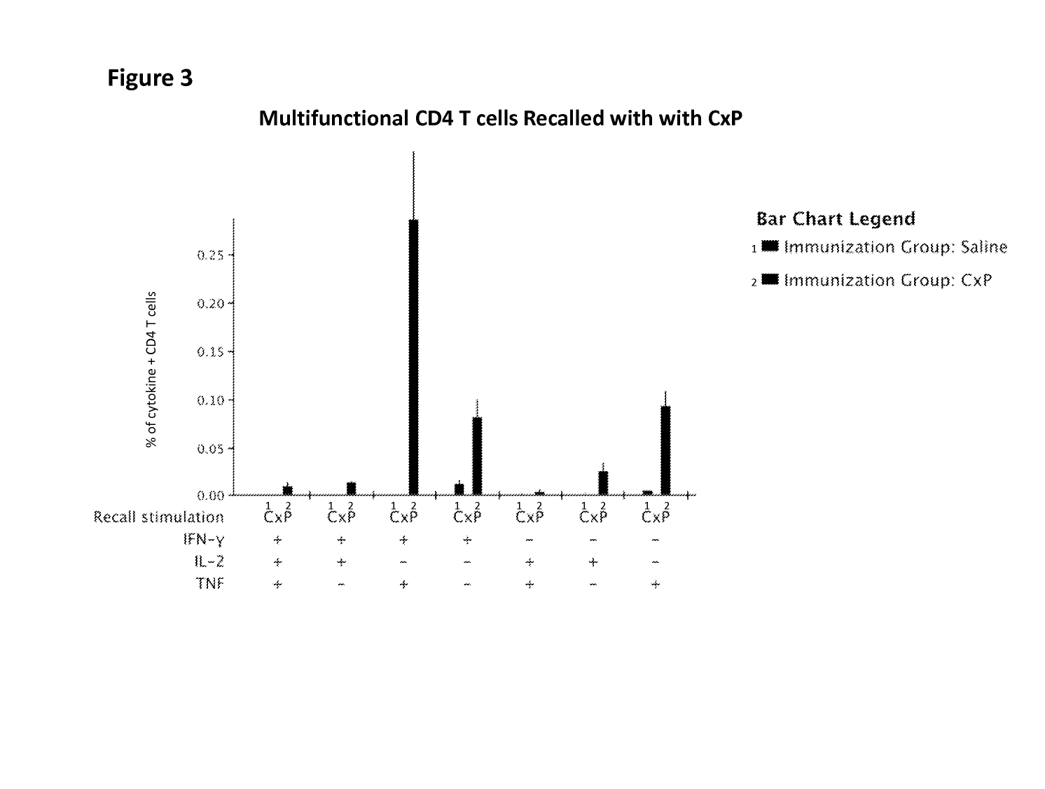

Flow cytometry analysis of mice immunized with CxP demonstrated a CD4+ T cell population that produced both IFNγ and TNF or a CD4+ T population that produced IFNγ alone in response to restimulation with CxP (FIG. 3). IFNγ was also secreted from spleen cell cultures immunized in vivo with the CxP polypeptide from L donovani then restimulated in vitro with lysates of L. infantum and L. major, 632 pg/ml and 106 pg/ml respectively, demonstrating cross-reactivity against these Leishmania species.

### Example 6

### Immunogenicity of the Fusion Polypeptide 821X

BALB/c mice were immunized according to the methods described and the cellular and humoral immune responses were analyzed. In addition a subset of mice were challenged with L donovani as described in Example 2 and the parasite burden was assessed.

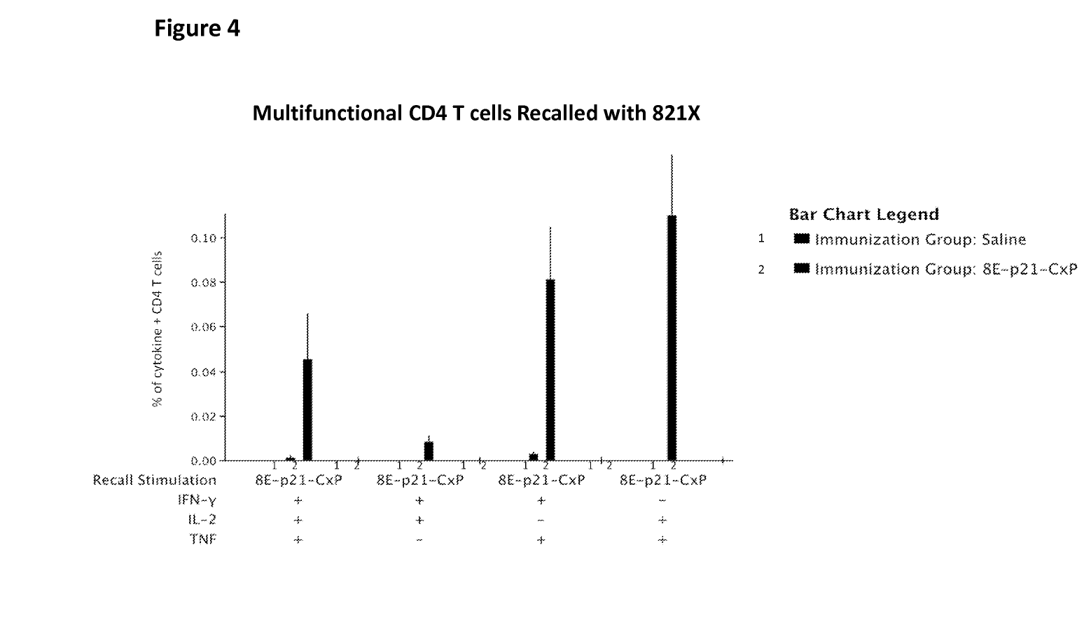

The 821X Fusion polypeptide comprises amino acids 509 to 660 of the carboxy-terminus of the putative mitochondrial HSP70 (8E or 8) polypeptide from L infantum, amino acids 1 to 191 of the p21 antigen polypeptide from Leishmania donovani, and amino acids 1 to 503 of the full length putative carboxypeptidase (CxP or X) polypeptide of L donovani. The data shows that mice immunized with 821X had a reduced L donovani burden in their livers, a mean of mean 1.88×10⁵ for animals immunized with 821X versus mean 4.06×10⁵ for unimmunized mice as determined by real time PCR. In addition as demonstrated in FIG. 4, mice immunized with 821X possessed a pluripotent CD4+ T cell population that in response to in vitro restimulation with the 821X polypeptide secreted IFNγ, TNF, or IL-2 alone or in combination. Further mice immunized with 821X possessed a CD4+ T cell population that produced both IFNγ and TNF in response to restimulation with the fusion polypeptide 821X or the individual polypeptide, CxP, of the 821X fusion polypeptide alone (FIG. 4). Incubation of spleen cells cultures from 821X immunized mice demonstrated IFNγ was specifically secreted in response in vitro stimulation (recall stimulation) with each individual polypeptide of the fusion; 8E restimulated cultures produced 1253 pg/ml, p21 restimulated cultures produced 6858 pg/ml and CxP restimulated cultures produced 683 pg/ml. IFNγ was also secreted by spleen cell cultures form mice immunized with the 821X fusion polypeptide in response to in vitro restimulation with an L. tropica lysate indicating cross-reactivity of the immune response to the 821X fusion polypeptide.

As would be recognized by the skilled artisan, these and other changes can be made to the embodiments of the invention in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled.

SEQUENCES

821X: mtHSP70₅₀₉₋₆₆₀ + p21₁₋₁₉₁ + CxP₁₋₅₀₃
8E (1 . . . 459) + p21 (460 . . . 1032) + CxP (1033 . . . 2541)
MW = 95,803 Daltons

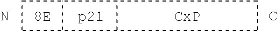

SEQ ID NO: 1 Polynucleotide encoding the 821X Fusion Polypeptide
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGAGCATTATCAAGGAGGACGACGCCGTGGGCTGCTACATGACGGTGACCCTCGTGGACGACACCAAGGTGGAGGGTACCATCTTC
ACCTACAATCCCAAGGAAGGCATCATAGTACTTCTGTCCCTCCGCGACGATCAGACGAACATGAAGCTGATCCGCACTCCATACATCAAAG
AGTTCAGTATTTCACACGCTGAGGAGGGAACGCACCTGCCTCCGGCACTGGACTCCTTCAACGAGCTTCCGTCCATGCATGCCGGCCGCGA
CAAGTCCATCTTCAAGCACGCCAGCACGCAGCTCAAGAACGCCGAGGCGAACCGCGAAAAGCACTTCAACTCTGTCACGACCGACACACCG
ATTGCCACACTCGATGCGTACCTCAAGCTCCTGCGGCTATACCCCTTCATTGAGTGGAACAGCGACGAGGGTGTCATCCAGGTCTCGGATA
CCGTCATTGTCGTAGGGGACCCCGACTGGCGGACGCCCAAGGCGATGCTGGTAGACGGCGCCCCTGAGAAGGACAGACCGCTCGTAGACCG
CCTGCAGGTTGCGCTCGGAAACGGCAAGAAGATGCAGGCCTACACACAACTGGAGAAGCTCTGCCAGAAGGTGTACAGATTGGCGCACCTT
CTGTCTCTCGGCGCTTGGGATTCCAAGACTATGATGCCCTCAAAGGGCGCAGCTGCCCGCGGTGCCGCCCTCGGCGAGCTCTACGGACTCA
TCGCTGAGATGATCACCAGCCCGAGCACGAAGGCGCTGCTGGACGAAGCAGAGACGGCCAAGGCCGAGCTCACTACTGTCCAGCAGGCGAA
CTTGCGCGAGCTCCGCCGCATGTACACCTCTCAAGCAGCGCTACCGACCGAGTTCAGTGTGCTCAAGACCAAGCTTTCGTCAACTACTCCG
CTTATCTGGGTTAAGTGCCGCAGCAACAACGACTTTGCGACTTTCCTGCCGGCGCTGAAGGAGATGATTGCGCTTGCGCGCAGGGAGGCGC
AGTATCGCTCTACTGCGACGGGCAAGCCTCTGTACGAGGCCCTGTTCAACCAGTACGAGAGCGGCATGACGCTGGAGACGCTGGAAAAAAT
CTTGCTCGATGTGAAGTCGTGGCTGCCGGAGCTGCTGCAGAAGATCCTGGCTGCACAGAGGGACGCGGGGCTGGAGGTGGTTGCGCCTGAG
GCGCCCTTTCCCAAGGACAAGCAGGAGGCTCTTAGCCGCCACCTCATGGAGGTGTGGGGCTTCGACTTCGAGTCAGGTCGGCTGGACGTCT
CTGAGCACCCGTTTATGGGCATGGTAAAGGAAGACTCGCGCATCACTACCGCCTACGACCTGCAGGACTTCACCAAGGGGCTCTTCGCGAC
GATCCACGAGACGGGCCACTCCAAGTACGAGACGAACTGCGGCCCGGTGGAGATGCGCGGCCAGCCGGTGTGCGAGGCACGCTCGATGACG
ATCCACGAGAGCCAGTCGCGCTTTGCCGAGGTTGTGATTGGCCACTCCAGCGCCTTCTTGGAGTTCCTCGTTCCACTGCTGAAGGAATACC
TCGGTGATCAGCCCGCATTCTCTCGGGAGAACGTGCGGCTGATGAACCAGACGGTGAAGCCTGGCTTCATCCGGATCCGGGCGGATGAGGT
GTGCTACCCGCTGCACATCTTGCTGCGCTACGAGATAGAGCGTGCACTCATCGAGGGCACGATGGAGGCAGAAGACATCCCTCGCGTGTGG
AACGAGAAGATGAAGGCATACCTGGGCCTGGAGACGGAGGGCCGCGACGAGATTGGCTGCCTGCAGGACATTCACTGGTCGATGGGCGCCT
TTGGCTACTTCCCGACGTACTCGCTTGGCTCCATGTTCGCGGCGCAGCTGATGGCGACGATCAAGAATGAGCTCGGTGAGGATACAGTGGA
CAAGTGCATCCGCACTGGCCAGATGGAGCCGATCTTTGAGAAGCAGAGGGAGAAGATCTGGAGCCAGGGATGCCTCTACAACACGGAAGAC
CTGATTGTCAAGGCGACCGGCGAAGCGCTGAACCCCAAGTACTTTCGCGAGTACCTGGAACGCCGCTACCTGCGCCAGGAGGAC
SEQ ID NO: 2 Amino Acid Sequence of the 821X Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMSIIKEDDAVGCYMTVTLVDDTKVEGTIF
TYNPKEGIIVLLSLRDDQTNMKLIRTPYIKEFSISHAEEGTHLPPALDSFNELPSMHAGRDKSIFKHASTQLKNAEANREKHFNSVTTDTP
IATLDAYLKLLRLYPFIEWNSDEGVIQVSDTVIVVGDPDWRTPKAMLVDGAPEKDRPLVDRLQVALGNGKKMQAYTQLEKLCQKVYRLAHL
LSLGAWDSKTMMPSKGAAARGAALGELYGLIAEMITSPSTKALLDEAETAKAELTTVQQANLRELRRMYTSQAALPTEFSVLKTKLSSTTP
LIWVKCRSNNDFATFLPALKEMIALARREAQYRSTATGKPLYEALFNQYESGMTLETLEKILLDVKSWLPELLQKILAAQRDAGLEVVAPE
APFPKDKQEALSRHLMEVWGFDFESGRLDVSEHPFMGMVKEDSRITTAYDLQDFTKGLFATIHETGHSKYETNCGPVEMRGQPVCEARSMT
IHESQSRFAEVVIGHSSAFLEFLVPLLKEYLGDQPAFSRENVRLMNQTVKPGFIRIRADEVCYPLHILLRYEIERALIEGTMEAEDIPRVW
NEKMKAYLGLETEGRDEIGCLQDIHWSMGAFGYFPTYSLGSMFAAQLMATIKNELGEDTVDKCIRTGQMEPIFEKQREKIWSQGCLYNTED
LIVKATGEALNPKYFREYLERRYLRQED
821XH: mtHSP70₅₀₉₋₆₆₀ + p21₁₋₁₉₁ + CxP₁₋₅₀₃ +H2BN₁₋₄₆
8E (1 . . . 459) + p21 (460 . . . 1032) + CxP (1033 . . . 2541) + H2Bn (2542 . . . 2679)
MW = 101,016 Daltons

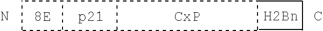

SEQ ID NO: 3 Polynucleotide encoding the 821XH Fusion Polypeptide
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGAGCATTATCAAGGAGGACGACGCCGTGGGCTGCTACATGACGGTGACCCTCGTGGACGACACCAAGGTGGAGGGTACCATCTTC
ACCTACAATCCCAAGGAAGGCATCATAGTACTTCTGTCCCTCCGCGACGATCAGACGAACATGAAGCTGATCCGCACTCCATACATCAAAG
AGTTCAGTATTTCACACGCTGAGGAGGGAACGCACCTGCCTCCGGCACTGGACTCCTTCAACGAGCTTCCGTCCATGCATGCCGGCCGCGA
CAAGTCCATCTTCAAGCACGCCAGCACGCAGCTCAAGAACGCCGAGGCGAACCGCGAAAAGCACTTCAACTCTGTCACGACCGACACACCG
ATTGCCACACTCGATGCGTACCTCAAGCTCCTGCGGCTATACCCCTTCATTGAGTGGAACAGCGACGAGGGTGTCATCCAGGTCTCGGATA
CCGTCATTGTCGTAGGGGACCCCGACTGGCGGACGCCCAAGGCGATGCTGGTAGACGGCGCCCCTGAGAAGGACAGACCGCTCGTAGACCG
CCTGCAGGTTGCGCTCGGAAACGGCAAGAAGATGCAGGCCTACACACAACTGGAGAAGCTCTGCCAGAAGGTGTACAGATTGGCGCACCTT
CTGTCTCTCGGCGCTTGGGATTCCAAGACTATGATGCCCTCAAAGGGCGCAGCTGCCCGCGGTGCCGCCCTCGGCGAGCTCTACGGACTCA
TCGCTGAGATGATCACCAGCCCGAGCACGAAGGCGCTGCTGGACGAAGCAGAGACGGCCAAGGCCGAGCTCACTACTGTCCAGCAGGCGAA
CTTGCGCGAGCTCCGCCGCATGTACACCTCTCAAGCAGCGCTACCGACCGAGTTCAGTGTGCTCAAGACCAAGCTTTCGTCAACTACTCCG
CTTATCTGGGTTAAGTGCCGCAGCAACAACGACTTTGCGACTTTCCTGCCGGCGCTGAAGGAGATGATTGCGCTTGCGCGCAGGGAGGCGC
AGTATCGCTCTACTGCGACGGGCAAGCCTCTGTACGAGGCCCTGTTCAACCAGTACGAGAGCGGCATGACGCTGGAGACGCTGGAAAAAAT
CTTGCTCGATGTGAAGTCGTGGCTGCCGGAGCTGCTGCAGAAGATCCTGGCTGCACAGAGGGACGCGGGGCTGGAGGTGGTTGCGCCTGAG
GCGCCCTTTCCCAAGGACAAGCAGGAGGCTCTTAGCCGCCACCTCATGGAGGTGTGGGGCTTCGACTTCGAGTCAGGTCGGCTGGACGTCT
CTGAGCACCCGTTTATGGGCATGGTAAAGGAAGACTCGCGCATCACTACCGCCTACGACCTGCAGGACTTCACCAAGGGGCTCTTCGCGAC
GATCCACGAGACGGGCCACTCCAAGTACGAGACGAACTGCGGCCCGGTGGAGATGCGCGGCCAGCCGGTGTGCGAGGCACGCTCGATGACG
ATCCACGAGAGCCAGTCGCGCTTTGCCGAGGTTGTGATTGGCCACTCCAGCGCCTTCTTGGAGTTCCTCGTTCCACTGAATACCTCGGTGA
TCAGCCCGCATTCTCTCGGGAGAACGTGCGGCTGATGAACCAGACGGTGAAGCCTGGCTTCATCCGGATCCGGGCGGATGAGGTGTGCTAC
CCGCTGCACATCTTGCTGCGCTACGAGATAGAGCGTGCACTCATCGAGGGCACGATGGAGGCAGAAGACATCCCTCGCGTGTGGAACGAGA
AGATGAAGGCATACCTGGGCCTGGAGACGGAGGGCCGCGACGAGATTGGCTGCCTGCAGGACATTCACTGGTCGATGGGCGCCTTTGGCTA
CTTCCCGACGTACTCGCTTGGCTCCATGTTCGCGGCGCAGCTGATGGCGACGATCAAGAATGAGCTCGGTGAGGATACAGTGGACAAGTGC
ATCCGCACTGGCCAGATGGAGCCGATCTTTGAGAAGCAGAGGGAGAAGATCTGGAGCCAGGGATGCCTCTACAACACGGAAGACCTGATTG
TCAAGGCGACCGGCGAAGCGCTGAACCCCAAGTACTTTCGCGAGTACCTGGAACGCCGCTACCTGCGCCAGGAGGACATGGCCTCTTCTCG
CTCTGCTCCCCGCAAGGCTTCCCACGCGCACAAGTCGCACCGCAAGCCGAAGCGCTCGTGGAACGTGTACGTGGGCCGCTCGCTGAAGGCG
ATCAACGCCCAGATGTCGATGTCGCACCGCACG
SEQ ID NO: 4 Amino Acid Sequence of the 821XH Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMSIIKEDDAVGCYMTVTLVDDTKVEGTIF
TYNPKEGIIVLLSLRDDQTNMKLIRTPYIKEFSISHAEEGTHLPPALDSFNELPSMHAGRDKSIFKHASTQLKNAEANREKHFNSVTTDTP
IATLDAYLKLLRLYPFIEWNSDEGVIQVSDTVIVVGDPDWRTPKAMLVDGAPEKDRPLVDRLQVALGNGKKTQAYTQLEKLCQKVYRLAHL
LSLGAWDSKTHMPSKGAAARGAALGELYGLIAEMITSPSTKALLDEAETAKAELTTVQQANLRELRRMYTSQAALPTEFSVLKTKLSSTTP
LIWVKCRSNNDFATFLPALKEMIALARREAQYRSTATGKPLYEALFNQYESGMTLETLEKILLDVKSWLPELLQKILAAQRDAGLEVVAPE
APFPKDKQEALSRHLMEVWGFDFESGRLDVSEHPFMGMVKEDSRITTAYDLQDFTKGLFATIHETGHSKYETNCGPVEMRGQPVCEARSMT
IHESQSRFAEVVIGHSSAFLEFLVPLLKEYLGDQPAFSRENVRLMNQTVKPGFIRIRADEVCYPLHILLRYEIERALIEGTMEAEDIPRVW
NEKMKAYLGLETEGRDEIGCLQDIHWSMGAFGYFPTYSLGSMFAAQLMATIKNELGEDTVDKCIRTGQMEPIFEKQREKIWSQGCLYNTED
LIVKATGEALNPKYFREYLERRYLRQEDMASSRSAPRKASHAHKSHRKPKRSWNVYVGRSLKAINAQMSMSHRT
821XA: mtHSP70₅₀₉₋₆₆₀ + p21₁₋₁₉₁ + CxP₁₋₅₀₃ + A2₂₃₋₂₃₆
8E (1 . . . 459) + p21 (460 . . . 1032) + CxP (1033 . . . 2541) + A2 (2542 . . . 3183)
MW = 115,539 Daltons

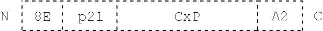

SEQ ID NO: 5 Polynucleotide encoding the 821XA Fusion Polypeptide
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGAGCATTATCAAGGAGGACGACGCCGTGGGCTGCTACATGACGGTGACCCTCGTGGACGACACCAAGGTGGAGGGTACCATCTTC
ACCTACAATCCCAAGGAAGGCATCATAGTACTTCTGTCCCTCCGCGACGATCAGACGAACATGAAGCTGATCCGCACTCCATACATCAAAG
AGTTCAGTATTTCACACGCTGAGGAGGGAACGCACCTGCCTCCGGCACTGGACTCCTTCAACGAGCTTCCGTCCATGCATGCCGGCCGCGA
CAAGTCCATCTTCAAGCACGCCAGCACGCAGCTCAAGAACGCCGAGGCGAACCGCGAAAAGCACTTCAACTCTGTCACGACCGACACACCG
ATTGCCACACTCGATGCGTACCTCAAGCTCCTGCGGCTATACCCCTTCATTGAGTGGAACAGCGACGAGGGTGTCATCCAGGTCTCGGATA
CCGTCATTGTCGTAGGGGACCCCGACTGGCGGACGCCCAAGGCGATGCTGGTAGACGGCGCCCCTGAGAAGGACAGACCGCTCGTAGACCG
CCTGCAGGTTGCGCTCGGAAACGGCAAGAAGATGCAGGCCTACACACAACTGGAGAAGCTCTGCCAGAAGGTGTACAGATTGGCGCACCTT
CTGTCTCTCGGCGCTTGGGATTCCAAGACTATGATGCCCTCAAAGGGCGCAGCTGCCCGCGGTGCCGCCCTCGGCGAGCTCTACGGACTCA
TCGCTGAGATGATCACCAGCCCGAGCACGAAGGCGCTGCTGGACGAAGCAGAGACGGCCAAGGCCGAGCTCACTACTGTCCAGCAGGCGAA
CTTGCGCGAGCTCCGCCGCATGTACACCTCTCAAGCAGCGCTACCGACCGAGTTCAGTGTGCTCAAGACCAAGCTTTCGTCAACTACTCCG
CTTATCTGGGTTAAGTGCCGCAGCAACAACGACTTTGCGACTTTCCTGCCGGCGCTGAAGGAGATGATTGCGCTTGCGCGCAGGGAGGCGC
AGTATCGCTCTACTGCGACGGGCAAGCCTCTGTACGAGGCCCTGTTCAACCAGTACGAGAGCGGCATGACGCTGGAGACGCTGGAAAAAAT
CTTGCTCGATGTGAAGTCGTGGCTGCCGGAGCTGCTGCAGAAGATCCTGGCTGCACAGAGGGACGCGGGGCTGGAGGTGGTTGCGCCTGAG
GCGCCCTTTCCCAAGGACAAGCAGGAGGCTCTTAGCCGCCACCTCATGGAGGTGTGGGGCTTCGACTTCGAGTCAGGTCGGCTGGACGTCT
CTGAGCACCCGTTTATGGGCATGGTAAAGGAAGACTCGCGCATCACTACCGCCTACGACCTGCAGGACTTCACCAAGGGGCTCTTCGCGAC
GATCCACGAGACGGGCCACTCCAAGTACGAGACGAACTGCGGCCCGGTGGAGATGCGCGGCCAGCCGGTGTGCGAGGCACGCTCGATGACG
ATCCACGAGAGCCAGTCGCGCTTTGCCGAGGTTGTGATTGGCCACTCCAGCGCCTTCTTGGAGTTCCTCGTTCCACTGCTGAAGGAATACC
TCGGTGATCAGCCCGCATTCTCTCGGGAGAACGTGCGGCTGATGAACCAGACGGTGAAGCCTGGCTTCATCCGGATCCGGGCGGATGAGGT
GTGCTACCCGCTGCACATCTTGCTGCGCTACGAGATAGAGCGTGCACTCATCGAGGGCACGATGGAGGCAGAAGACATCCCTCGCGTGTGG
AACGAGAAGATGAAGGCATACCTGGGCCTGGAGACGGAGGGCCGCGACGAGATTGGCTGCCTGCAGGACATTCACTGGTCGATGGGCGCCT
TTGGCTACTTCCCGACGTACTCGCTTGGCTCCATGTTCGCGGCGCAGCTGATGGCGACGATCAAGAATGAGCTCGGTGAGGATACAGTGGA
CAAGTGCATCCGCACTGGCCAGATGGAGCCGATCTTTGAGAAGCAGAGGGAGAAGATCTGGAGCCAGGGATGCCTCTACAACACGGAAGAC
CTGATTGTCAAGGCGACCGGCGAAGCGCTGAACCCCAAGTACTTTCGCGAGTACCTGGAACGCCGCTACCTGCGCCAGGAGGACAGCGCCT
CCGCTGAGCCGCACAAGGCGGCCGTTGACGTCGGCCCGCTGAGCGTTGGCCCGCAGAGCGTCGGCCCGCTGAGCGTTGGCCCGCAGGCGGT
TGGCCCGCTGAGCGTTGGCCCGCAGAGCGTCGGCCCGCTGAGCGTTGGCCCGCAGGCGGTTGGCCCGCTGAGCGTTGGCCCGCAGAGCGTT
GGCCCGCTGAGCGTTGGCCCGCTGAGCGTTGGCCCGCAGAGCGTTGGCCCGCTGAGCGTTGGCAGCCAGAGCGTCGGCCCGCTGAGCGTTG
GTCCGCAGAGCGTCGGCCCGCTGAGCGTTGGCCCGCAGGCGGTTGGCCCGCTGAGCGTTGGCCCGCAGAGCGTCGGCCCGCTGAGCGTTGG
CCCGCAGGCGGTTGGCCCGCTGAGCGTTGGCCCGCAGAGCGTTGGCCCGCTGAGCGTTGGCCCGCAGAGCGTTGGCCCGCTGAGCGTTGGC
AGCCAGAGCGTCGGCCCGCTGAGCGTTGGTCCGCAGAGCGTCGGCCCGCTGAGCGTTGGCCCGCAGAGCGTCGGCCCGCTGAGCGTTGGCC
CGCAGAGCGTCGGCCCGCTGAGCGTTGGTCCGCAGAGCGTTGGCCCGCTGAGCGTTGGCCCGCAGAGCGTTGACGTTAGCCCGGTGAGC
SEQ ID NO: 6 Amino Acid Sequence of the 821XA Fusion Polypeptide
MKDKATGKTQNITITANGGLSKEQIEQMIRDSEQHAEADRVKRELVEVRNNAETQLTTAERQLGEWKVYSDAEKENVKTLVAELRKAMENP
NVAKDDLAAATDKLQKAVMECGRTEYQQAAAANSGSTSNSGEQQQQQGQGEQQQQQNSEEKKMSIIKEDDAVGCYMTVTLVDDTKVEGTIF
TYNPKEGIIVLLSLRDDQTNMKLIRTPYIKEFSISHAEEGTHLPPALDSFNELPSMHAGRDKSIFKHASTQLKNAEANREKHFNSVTTDTP
IATLDAYLKLLRLYPFIEWNSDEGVIQVSDTVIVVGDPDWRTPKAMLVDGAPEKDRPLVDRLQVALGNGKKMQAYTQLEKLCQKVYRLAHL
LSLGAWDSKTMMPSKGAAARGAALGELYGLIAEMITSPSTKALLDEAETAKAELTTVQQANLRELRRMYTSQAALPTEFSVLKTKLSSTTP
LIWVKCRSNNDFATFLPALKEMIALARREAQYRSTATGKPLYEALFNQYESGMTLETLEKILLDVKSWLPELLQKILAAQRDAGLEVVAPE
APFPKDKQEALSRHLMEVWGFDFESGRLDVSEHPFMGMVKEDSRITTAYDLQDFTKGLFATIHETGHSKYETNCGPVEMRGQPVCEARSMT
IHESQSRFAEVVIGHSSAFLEFLVPLLKEYLGDQPAFSRENVRLMNQTVKPGFIRIRADEVCYPLHILLRYEIERALIEGTMEAEDIPRVW
NEKMKAYLGLETEGRDEIGCLQDIHWSMGAFGYFPTYSLGSMFAAQLMATIKNELGEDTVDKCIRTGQMEPIFEKQREKIWSQGCLYNTED
LIVKATGEALNPKYFREYLERRYLRQEDSASAEPHKAAVDVGPLSVGPQSVGPLSVGPOAVGPLSVGPOSVGPLSVGPOAVGPLSVGPQSV
GPLSVGPLSVGPQSVGPLSVGSQSVGPLSVGPQSVGPLSVGPQAVGPLSVGPQSVGPLSVGPQAVGPLSVGPQSVGPLSVGPQSVGPLSVG
SQSVGPLSVGPQSVGPLSVGPQSVGPLSVGPQSVGPLSVGPQSVGPLSVGPQSVDVSPVS
821NA: mtHSP70₅₀₉₋₆₆₀ + p21₁₋₁₉₁ + NH₁₋₃₁₄ + A2₂₃₋₂₃₆
8E (1 . . . 459) + p21 (460 . . . 1032) + NH (1033 . . . 1974) + A2 (1975 . . . 2616)
MW = 92,582 Daltons

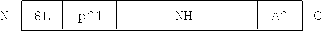

SEQ ID NO: 7 Polynucleotide encoding the 821NA Fusion Polypeptide
ATGAAGGACAAGGCGACGGGCAAGACGCAGAACATCACGATCACGGCGAACGGCGGGCTGTCGAAGGAGCAGATCGAGCAGATGATCCGC
GACTCGGAGCAGCACGCGGAGGCCGACCGCGTGAAGCGCGAGCTTGTGGAGGTGCGCAACAACGCGGAGACGCAGCTGACAACGGCGGAG
AGGCAGCTCGGCGAGTGGAAGTACGTGAGCGATGCGGAGAAGGAGAACGTGAAGACGCTGGTGGCGGAGCTGCGCAAGGCGATGGAGAAC
CCGAACGTCGCGAAGGATGACCTTGCGGCTGCGACGGACAAGCTGCAGAAGGCTGTGATGGAGTGCGGCCGCACAGAGTACCAGCAGGCT
GCCGCGGCCAACTCCGGCAGCACCAGCAACTCCGGTGAGCAGCAGCAGCAGCAGGGCCAAGGTGAGCAGCAGCAGCAGCAGAACAGCGAA
GAGAAGAAGATGAGCATTATCAAGGAGGACGACGCCGTGGGCTGCTACATGACGGTGACCCTCGTGGACGACACCAAGGTGGAGGGTACC
ATCTTCACCTACAATCCCAAGGAAGGCATCATAGTACTTCTGTCCCTCCGCGACGATCAGACGAACATGAAGCTGATCCGCACTCCATAC
ATCAAAGAGTTCAGTATTTCACACGCTGAGGAGGGAACGCACCTGCCTCCGGCACTGGACTCCTTCAACGAGCTTCCGTCCATGCATGCC
GGCCGCGACAAGTCCATCTTCAAGCACGCCAGCACGCAGCTCAAGAACGCCGAGGCGAACCGCGAAAAGCACTTCAACTCTGTCACGACC
GACACACCGATTGCCACACTCGATGCGTACCTCAAGCTCCTGCGGCTATACCCCTTCATTGAGTGGAACAGCGACGAGGGTGTCATCCAG
GTCTCGGATACCGTCATTGTCGTAGGGGACCCCGACTGGCGGACGCCCAAGGCGATGCTGGTAGACGGCGCCCCTGAGAAGGACAGACCG
CTCGTAGACCGCCTGCAGGTTGCGCTCGGAAACGGCAAGAAGATGCCGCGCAAGATTATTCTCGATTGTGATCCCGGGATCGATGATGCC
GTGGCCATCTTTCTCGCCCACGGCAACCCGGAGGTCGAGCTGCTGGCCATTACGACGGTGGTGGGCAACCAGACCCTGGAGAAGGTGACC
CGGAACGCGCGGCTGGTAGCTGACGTAGCCGGCATCGTTGGTGTGCCCGTCGCGGCTGGTTGCACCAAGCCCCTCGTGCGCGGTGTGCGG
AATGCCTCTCAGATTCATGGCGAAACCGGCATGGGTAACGTCTCCTACCCACCAGAGTTCAAGACAAAGTTGGACGGCCGTCATGCAGTG
CAGCTGATCATCGACCTTATCATGTCGCACGAGCCGAAGACGATCACGCTTGTGCCTACGGGTGGCCTGACGAACATTGCGATGGCTGTC
CGTCTTGAGCCGCGCATCGTGGACCGTGTGAAGGAGGTGGTTCTGATGGGTGGCGGCTACCATACTGGTAATGCGTCCCCTGTAGCGGAG
TTCAACGTCTTCGTCGACCCGGAGGCGGCGCACATTGTGTTCAACGAGAGCTGGAACGTAACGATGGTGGGGCTGGACCTAACGCACCAG
GCACTCGCCACGCCGGCGGTCCAGAAGCGAGTGAAGGAGGTGGGCACGAAGCCGGCTGCCTTCATGCTGCAGATTTTGGACTTTTACACG
AAGGTGTACGAAAAGGAGCGCAACACGTACGCGACGGTGCACGATCCCTGCGCTGTGGCGTACGTGATTGACCCCACCGTGATGACGACG
GAGCAAGTGCCAGTGGACATCGAGCTCAATGGGGCACTGACGACTGGGATGACGGTCGCGGACTTCCGCTACCCACGGCCAAAGCACTGC
CACACGCAGGTGGCTGTGAAGCTGGACTTCGACAAGTTTTGGTGCCTCGTGATTGACGCACTCAAGCGCATCGGCGATCCTCAAAGCGCC
TCCGCTGAGCCGCACAAGGCGGCCGTTGACGTCGGCCCGCTGAGCGTTGGCCCGCAGAGCGTCGGCCCGCTGAGCGTTGGCCCGCAGGCG
GTTGGCCCGCTGAGCGTTGGCCCGCAGAGCGTCGGCCCGCTGAGCGTTGGCCCGCAGGCGGTTGGCCCGCTGAGCGTTGGCCCGCAGAGC
GTTGGCCCGCTGAGCGTTGGCCCGCTGAGCGTTGGCCCGCAGAGCGTTGGCCCGCTGAGCGTTGGCAGCCAGAGCGTCGGCCCGCTGAGC
GTTGGTCCGCAGAGCGTCGGCCCGCTGAGCGTTGGCCCGCAGGCGGTTGGCCCGCTGAGCGTTGGCCCGCAGAGCGTCGGCCCGCTGAGC
GTTGGCCCGCAGGCGGTTGGCCCGCTGAGCGTTGGCCCGCAGAGCGTTGGCCCGCTGAGCGTTGGCCCGCAGAGCGTTGGCCCGCTGAGC
GTTGGCAGCCAGAGCGTCGGCCCGCTGAGCGTTGGTCCGCAGAGCGTCGGCCCGCTGAGCGTTGGCCCGCAGAGCGTCGGCCCGCTGAGC
GTTGGCCCGCAGAGCGTCGGCCCGCTGAGCGTTGGTCCGCAGAGCGTTGGCCCGCTGAGCGTTGGCCCGCAGAGCGTTGACGTTAGCCCG
GTGAGC
SEQ ID NO: 8 Amino Acid Sequence of the 821NA Fusion Polypeptide
MKDKATGKTQNITITANGGLSKEQIEQMIRDSEQHAEADRVKRELVEVRNNAETQLTTAERQLGEWKYVSDAEKENVKTLVAELRKAMEN
PNVAKDDLAAATDKLQKAVMECGRTEYQQAAAANSGSTSNSGEQQQQQGQGEQQQQQNSEEKKMSIIKEDDAVGCYMTVTLVDDTKVEGT
IFTYNPKEGIIVLLSLRDDQTNMKLIRTPYIKEFSISHAEEGTHLPPALDSFNELPSMHAGRDKSIFKHASTQLKNAEANREKHFNSVTT
DTPIATLDAYLKLLRLYPFIEWNSDEGVIOVSDTVIVVGDPDWRTPKAMLVDGAPEKDRPLVDRLQVALGNGKKMPRKIILDCDPGIDDA
VAIFLAHGNPEVELLAITTVVGNQTLEKVTRNARLVADVAGIVGVPVAAGCTKPLVRGVRNASQIHGETGMGNVSYPPEFKTKLDGRHAV
QLIIDLIMSHEPKTITLVPTGGLTNIAMAVRLEPRIVDRVKEVVLMGGGYHTGNASPVAEFNVFVDPEAAHIVFNESWNVTMVGLDLTHQ
ALATPAVQKRVKEVGTKPAAFMLQILDFYTKVYEKERNTYATVHDPCAVAYVIDPTVMTTEQVPVDIELNGALTTGMTVADFRYPRPKHC
HTQVAVKLDFDKFWCLVIDALKRIGDPQSASAEPHKAAVDVGPLSVGPQSVGPLSVGPQAVGPLSVGPQSVGPLSVGPQAVGPLSVGPQS
VGPLSVGPLSVGPQSVGPLSVGSQSVGPLSVGPQSVGPLSVGPQAVGPLSVGPQSVGPLSVGPQAVGPLSVGPQSVGPLSVGPQSVGPLS
VGSQSVGPLSVGPQSVGPLSVGPQSVGPLSVGPQSVGPLSVGPQSVGPLSVGPQSVDVSPVS
NXH: NH₁₋₃₁₄ + CxP₁₋₅₀₃ + H2B₁₋₄₆
Nh (1 . . . 942) + CxP (943 . . . 2451) + H2Bn (2452 . . . 2589)
MW = 96,629 Daltons

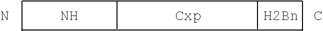

SEQ ID NO: 9 Polynucleotide encoding the NXH Fusion Polypeptide
ATGCCGCGCAAGATTATTCTCGATTGTGATCCCGGGATCGATGATGCCGTGGCCATCTTTCTCGCCCACGGCAACCCGGAGGTCGAGCTGC
TGGCCATTACGACGGTGGTGGGCAACCAGACCCTGGAGAAGGTGACCCGGAACGCGCGGCTGGTAGCTGACGTAGCCGGCATCGTTGGTGT
GCCCGTCGCGGCTGGTTGCACCAAGCCCCTCGTGCGCGGTGTGCGGAATGCCTCTCAGATTCATGGCGAAACCGGCATGGGTAACGTCTCC
TACCCACCAGAGTTCAAGACAAAGTTGGACGGCCGTCATGCAGTGCAGCTGATCATCGACCTTATCATGTCGCACGAGCCGAAGACGATCA
CGCTTGTGCCTACGGGTGGCCTGACGAACATTGCGATGGCTGTCCGTCTTGAGCCGCGCATCGTGGACCGTGTGAAGGAGGTGGTTCTGAT
GGGTGGCGGCTACCATACTGGTAATGCGTCCCCTGTAGCGGAGTTCAACGTCTTCGTCGACCCGGAGGCGGCGCACATTGTGTTCAACGAG
AGCTGGAACGTAACGATGGTGGGGCTGGACCTAACGCACCAGGCACTCGCCACGCCGGCGGTCCAGAAGCGAGTGAAGGAGGTGGGCACGA
AGCCGGCTGCCTTCATGCTGCAGATTTTGGACTTTTACACGAAGGTGTACGAAAAGGAGCGCAACACGTACGCGACGGTGCACGATCCCTG
CGCTGTGGCGTACGTGATTGACCCCACCGTGATGACGACGGAGCAAGTGCCAGTGGACATCGAGCTCAATGGGGCACTGACGACTGGGATG
ACGGTCGCGGACTTCCGCTACCCACGGCCAAAGCACTGCCACACGCAGGTGGCTGTGAAGCTGGACTTCGACAAGTTTTGGTGCCTCGTGA
TTGACGCACTCAAGCGCATCGGCGATCCTCAAATGCAGGCCTACACACAACTGGAGAAGCTCTGCCAGAAGGTGTACAGATTGGCGCACCT
TCTGTCTCTCGGCGCTTGGGATTCCAAGACTATGATGCCCTCAAAGGGCGCAGCTGCCCGCGGTGCCGCCCTCGGCGAGCTCTACGGACTC
ATCGCTGAGATGATCACCAGCCCGAGCACGAAGGCGCTGCTGGACGAAGCAGAGACGGCCAAGGCCGAGCTCACTACTGTCCAGCAGGCGA
ACTTGCGCGAGCTCCGCCGCATGTACACCTCTCAAGCAGCGCTACCGACCGAGTTCAGTGTGCTCAAGACCAAGCTTTCGTCAACTACTCC
GCTTATCTGGGTTAAGTGCCGCAGCAACAACGACTTTGCGACTTTCCTGCCGGCGCTGAAGGAGATGATTGCGCTTGCGCGCAGGGAGGCG
CAGTATCGCTCTACTGCGACGGGCAAGCCTCTGTACGAGGCCCTGTTCAACCAGTACGAGAGCGGCATGACGCTGGAGACGCTGGAAAAAA
TCTTGCTCGATGTGAAGTCGTGGCTGCCGGAGCTGCTGCAGAAGATCCTGGCTGCACAGAGGGACGCGGGGCTGGAGGTGGTTGCGCCTGA
GGCGCCCTTTCCCAAGGACAAGCAGGAGGCTCTTAGCCGCCACCTCATGGAGGTGTGGGGCTTCGACTTCGAGTCAGGTCGGCTGGACGTC
TCTGAGCACCCGTTTATGGGCATGGTAAAGGAAGACTCGCGCATCACTACCGCCTACGACCTGCAGGACTTCACCAAGGGGCTCTTCGCGA
CGATCCACGAGACGGGCCACTCCAAGTACGAGACGAACTGCGGCCCGGTGGAGATGCGCGGCCAGCCGGTGTGCGAGGCACGCTCGATGAC
GATCCACGAGAGCCAGTCGCGCTTTGCCGAGGTTGTGATTGGCCACTCCAGCGCCTTCTTGGAGTTCCTCGTTCCACTGCTGAAGGAATAC
CTCGGTGATCAGCCCGCATTCTCTCGGGAGAACGTGCGGCTGATGAACCAGACGGTGAAGCCTGGCTTCATCCGGATCCGGGCGGATGAGG
TGTGCTACCCGCTGCACATCTTGCTGCGCTACGAGATAGAGCGTGCACTCATCGAGGGCACGATGGAGGCAGAAGACATCCCTCGCGTGTG
GAACGAGAAGATGAAGGCATACCTGGGCCTGGAGACGGAGGGCCGCGACGAGATTGGCTGCCTGCAGGACATTCACTGGTCGATGGGCGCC
TTTGGCTACTTCCCGACGTACTCGCTTGGCTCCATGTTCGCGGCGCAGCTGATGGCGACGATCAAGAATGAGCTCGGTGAGGATACATCTA
CGTGGACAAGTGCATCCGCACTGGCCAGATGGAGCCGATCTTTGAGAAGCAGAGGGAGAAGATCTGGAGCCAGGGATGCCAACACGGAAGA
CCTGATTGTCAAGGCGACCGGCGAAGCGCTGAACCCCAAGTACTTTCGCGAGTACCTGGAACGCCGCTACCTGCGCCAGGAGGACATGGCC
TCTTCTCGCTCTGCTCCCCGCAAGGCTTCCCACGCGCACAAGTCGCACCGCAAGCCGAAGCGCTCGTGGAACGTGTACGTGGGCCGCTCGC
TGAAGGCGATCAACGCCCAGATGTCGATGTCGCACCGCACG
SEQ ID NO: 10 Amino Acid Sequence of the NXH Fusion Polypeptide
MPRKIILDCDPGIDDAVAIFLAHGNPEVELLAITTVVGNQTLEKVTRNARLVADVAGIVGVPVAAGCTKPLVRGVRNASQIHGETGMGNVS
YPPEFKTKLDRGHAVQLIIDLIMSHEPKTITLVPTGGLTNIAMAVRLEPRIVDRVKEVVLMGGGYHTGNASPVAEFNVFVDPEAAHIVFNE
SWNVTMVGLDLTHQALATPAVQKRVKEVGTKPAAFMLQILDFYTKVYEKERNTYATVHDPCAVAYVIDPTVMTTEQVPVDIELNGALTTGM
TVADFRYPRPKHCHTQVAVKLDFDKFWCLVIDALKRIGDPQMQAYTQLEKLCQKVYRLAHLLSLGAWDSKTMMPSKGAAARGAALGELYGL
IAEMITSPSTKALLDEAETAKAELTTVQQANLRELRRMYTSQAALPTEFSVLKTKLSSTTPLIWVKCRSNNDFATFLPALKEMIALARREA
QYRSTATGKPLYEALFNQYESGMTLETLEKILLDVKSWLPELLQKILAAQRDAGLEVVAPEAPFPKDKQEALSRHLMEVWGFDFESGRLDV
SEHPFMGMVKEDSRITTAYDLQDFTKGLFATIHETGHSKYETNCGPVEMRGQPVCEARSMTIHESQSRFAEVVIGHSSAFLEFLVPLLKEY
LGDQPAFSRENVRLMNQTVKPGFIRIRADEVCYPLHILLRYEIERALIEGTMEAEDIPRVWNEKMKAYLGLETEGRDEIGCLQDIHWSMGA
FGYFPTYSLGSMFAAQLMATIKNELGEDTVDKCIRTGQMEPIFEKQREKIWSQGCLYNTEDLIVKATGEALNPKYFREYLERRYLRQEDMA
SSRSAPRKASHAHKSHRKPKRSWNVYVGRSLKAINAQMSMSHRT
TXL (TSA_{full length L major 1-199} + CXP₁₋₅₀₃ + LeiF₁₋₂₂₆ amino terminus L major)
TXL (1-597) + CxP₁₋₅₀₃ (598-2112) + LeiF₁₋₂₂₆ (2113-2796)
MW = MW = 105,134 Daltons

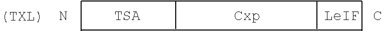

SEQ ID NO: 11 Polynucleotide encoding the TXL Fusion Polypeptide (underline = linker)
ATGTCCTGCGGTAACGCCAAGATCAACTCTCCCGCGCCGTCCTTCGAGGAGGTGGCGCTCATGCCCAACGGCAGCTTCAAGAAGATCAGCC
TCTCCTCCTACAAGGGCAAGTGGGTCGTGCTCTTCTTCTACCCGCTCGACTTCACCTTCGTGTGCCCGACAGAGGTCATCGCGTTCTCCGA
CAGCGTGAGTCGCTTCAACGAGCTCAACTGCGAGGTCCTCGCGTGCTCGATAGACAGCGAGTACGCGCACCTGCAGTGGACGCTGCAGGAC
CGCAAGAAGGGCGGCCTCGGGACCATGGCGATCCCAATGCTAGCCGACAAGACCAAGAGCATCGCTCGTTCCTACGGCGTGCTGGAGGAGA
GCCAGGGCGTGGCCTACCGCGGTCTCTTCATCATCGACCCCCATGGCATGCTGCGTCAGATCACCGTCAATGACATGCCGGTGGGCCGCAG
CGTGGAGGAGGTTCTACGCCTGCTGGAGGCTTTTCAGTTCGTGGAGAAGCACGGCGAGGTGTGCCCCGCGAACTGGAAGAAGGGCGCCCCC
ACGATGAAGCCGGAACCGAATGCGTCTGTCGAGGGATACTTCAGCAAGCAGGGATCCATGCAGGCCTACACACAACTGGAGAAGCTCTGCC
AGAAGGTGTACAGATTGGCGCACCTTCTGTCTCTCGGCGCTTGGGATTCCAAGACTATGATGCCCTCAAAGGGCGCAGCTGCCCGCGGTGC
CGCCCTCGGCGAGCTCTACGGACTCATCGCTGAGATGATCACCAGCCCGAGCACGAAGGCGCTGCTGGACGAAGCAGAGACGGCCAAGGCC
GAGCTCACTACTGTCCAGCAGGCGAACTTGCGCGAGCTCCGCCGCATGTACACCTCTCAAGCAGCGCTACCGACCGAGTTCAGTGTGCTCA
AGACCAAGCTTTCGTCAACTACTCCGCTTATCTGGGTTAAGTGCCGCAGCAACAACGACTTTGCGACTTTCCTGCCGGCGCTGAAGGAGAT
GATTGCGCTTGCGCGCAGGGAGGCGCAGTATCGCTCTACTGCGACGGGCAAGCCTCTGTACGAGGCCCTGTTCAACCAGTACGAGAGCGGC
ATGACGCTGGAGACGCTGGAAAAAATCTTGCTCGATGTGAAGTCGTGGCTGCCGGAGCTGCTGCAGAAGATCCTGGCTGCACAGAGGGACG
CGGGGCTGGAGGTGGTTGCGCCTGAGGCGCCCTTTCCCAAGGACAAGCAGGAGGCTCTTAGCCGCCACCTCATGGAGGTGTGGGGCTTCGA
CTTCGAGTCAGGTCGGCTGGACGTCTCTGAGCACCCGTTTATGGGCATGGTAAAGGAAGACTCGCGCATCACTACCGCCTACGACCTGCAG
GACTTCACCAAGGGGCTCTTCGCGACGATCCACGAGACGGGCCACTCCAAGTACGAGACGAACTGCGGCCCGGTGGAGATGCGCGGCCAGC
CGGTGTGCGAGGCACGCTCGATGACGATCCACGAGAGCCAGTCGCGCTTTGCCGAGGTTGTGATTGGCCACTCCAGCGCCTTCTTGGAGTT
CCTCGTTCCACTGCTGAAGGAATACCTCGGTGATCAGCCCGCATTCTCTCGGGAGAACGTGCGGCTGATGAACCAGACGGTGAAGCCTGGC
TTCATCCGGATCCGGGCGGATGAGGTGTGCTACCCGCTGCACATCTTGCTGCGCTACGAGATAGAGCGTGCACTCATCGAGGGCACGATGG
AGGCAGAAGACATCCCTCGCGTGTGGAACGAGAAGATGAAGGCATACCTGGGCCTGGAGACGGAGGGCCGCGACGAGATTGGCTGCCTGCA
GGACATTCACTGGTCGATGGGCGCCTTTGGCTACTTCCCGACGTACTCGCTTGGCTCCATGTTCGCGGCGCAGCTGATGGCGACGATCAAG
AATGAGCTCGGTGAGGATACAGTGGACAAGTGCATCCGCACTGGCCAGATGGAGCCGATCTTTGAGAAGCAGAGGGAGAAGATCTGGAGCC
AGGGATGCCTCTACAACACGGAAGACCTGATTGTCAAGGCGACCGGCGAAGCGCTGAACCCCAAGTACTTTCGCGAGTACCTGGAACGCCG
CTACCTGCGCCAGGAGGACGAATTCATGGCGCAGAATGATAAGATCGCCCCCCAGGACCAGGACTCCTTCCTCGATGACCAGCCCGGCGTT
CGCCCGATCCCGTCCTTCGACGACATGCCGCTGCACCAGAACCTGCTGCGTGGCATCTACTCGTACGGGTTCGAGAAGCCGTCCAGCATCC
AGCAGCGCGCGATAGCCCCCTTCACGCGCGGCGGCGACATCATCGCGCAGGCCCAGTCCGGTACCGGCAAGACGGGTGCCTTCTCCATCGG
TCTGCTGCAGCGCCTGGACTTCCGCCACAACCTGATCCAGGGCCTCGTGCTCTCCCCCACTCGCGAGCTGGCCCTGCAGACGGCGGAGGTG
ATCAGCCGCATCGGTGAGTTCCTGTCGAACAGCTCCAAGTTCTGCGAGACCTTTGTCGGCGGCACGCGCGTGCAGGATGACCTGCGCAAGC
TGCAGGCCGGCGTCATCGTTGCCGTGGGCACGCCGGGCCGCGTGTCCGACGTGATCAAGCGTGGCGCGCTGCGCACAGAGTCGCTGCGCGT
TGGTGCTCGACGAGGCTGATGAGATGCTGTCTCAGGGCTTCGCGGACCAGATTTACGAGATCTTCCGCTTCCTGCCGAAGGACATCCAGGT
CGCGCTCTTCTCCGCCACGATGCCGGAGGAGGTACTGGAGCTGACGAAGAAGTTCATGCGCGAC
SEQ ID NO: 12 Amino Acid Sequence of the TXL Fusion Polypeptide
MSCGNAKINSPAPSFEEVALMPNGSFKKISLSSYKGKWVVLFFYPLDFTFVCPTEVIAFSDSVSRFNELNCEVLACSIDSEYAHLQWTLQD
RKKGGLGTMAIPMLADKTKSIARSYGVLEESQGVAYRGLFIIDPHGMLRQITVNDMPVGRSVEEVLRLLEAFQFVEKHGEVCPANWKKGAP
TMKPEPNASVEGYFSKQGSMQAYTQLEKLCQKVYRLAHLLSLGAWDSKTMMPSKGAAARGAALGELYGLIAEMITSPSTKALLDEAETAKA
ELTTVQQANLRELRRMYTSQAALPTEFSVLKTKLSSTTPLIWVKCRSNNDFATFLPALKEMIALARREAQYRSTATGKPLYEALFNQYESG
MTLETLEKILLDVKSWLPELLQKILAAQRDAGLEVVAPEAPFPKDKQEALSRHLMEVWGFDFESGRLDVSEHPFMGMVKEDSRITTAYDLQ
DFTKGLFATIHETGHSKYETNCGPVEMRGQPVCEARSMTIHESQSRFAEVVIGHSSAFLEFLVPLLKEYLGDQPAFSRENVRLMNQTVKPG
FIRIRADEVCYPLHILLRYEIERALIEGTMEAEDIPRVWNEKMKAYLGLETEGRDEIGCLQDIHWSMGAFGYFPTYSLGSMFAAQLMATIK
NELGEDTVDKCIRTGQMEPIFEKQREKIWSQGCLYNTEDLIVKATGEALNPKYFREYLERRYLRQEDEFMAQNDKIAPQDQDSFLDDQPGV
RPIPSFDDMPLHQNLLRGIYSYGFEKPSSIQQRAIAPFTRGGDIIAQAQSGTGKTGAFSIGLLQRLDFRHNLIQGLVLSPTRELALQTAEV
ISRIGEFLSNSSKFCETFVGGTRVQDDLRKLQAGVIVAVGTPGRVSDVIKRGALRTESLRVLVLDEADEMLSQGFADQIYEIFRFLPKDIQ
VALFSATMPEEVLELTKKFMRD
8XHA (8E mtHSP70₅₀₉₋₆₆₀ + CxP₁₋₅₀₃ + H2B₁₋₄₆ + A2₂₃₋₂₃₆₎
8E (1-459) + CxP (460-1974) + H2B(1975-2124) + A2(2125-2766)
MW = 99,697 Daltons

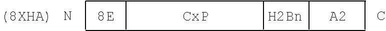

SEQ ID NO: 13 Polynucleotide encoding the 8XHA Fusion Polypeptide (underline = linker)
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGACTAGTATGCAGGCCTACACACAACTGGAGAAGCTCTGCCAGAAGGTGTACAGATTGGCGCACCTTCTGTCTCTCGGCGCTTGGGAT
TCCAAGACTATGATGCCCTCAAAGGGCGCAGCTGCCCGCGGTGCCGCCCTCGGCGAGCTCTACGGACTCATCGCTGAGATGATCACCAGCC
CGAGCACGAAGGCGCTGCTGGACGAAGCAGAGACGGCCAAGGCCGAGCTCACTACTGTCCAGCAGGCGAACTTGCGCGAGCTCCGCCGCAT
GTACACCTCTCAAGCAGCGCTACCGACCGAGTTCAGTGTGCTCAAGACCAAGCTTTCGTCAACTACTCCGCTTATCTGGGTTAAGTGCCGC
AGCAACAACGACTTTGCGACTTTCCTGCCGGCGCTGAAGGAGATGATTGCGCTTGCGCGCAGGGAGGCGCAGTATCGCTCTACTGCGACGG
GCAAGCCTCTGTACGAGGCCCTGTTCAACCAGTACGAGAGCGGCATGACGCTGGAGACGCTGGAAAAAATCTTGCTCGATGTGAAGTCGTG
GCTGCCGGAGCTGCTGCAGAAGATCCTGGCTGCACAGAGGGACGCGGGGCTGGAGGTGGTTGCGCCTGAGGCGCCCTTTCCCAAGGACAAG
CAGGAGGCTCTTAGCCGCCACCTCATGGAGGTGTGGATGGGCATGGTGGCTTCGACTTCGAGTCAGGTCGGCTGGACGTCTCTGAGCACCC
GTTTAAAGGAAGACTCGCGCATCACTACCGCCTACGACCTGCAGGACTTCACCAAGGGGCTCTTCGCGACGATCCACGAGACGGGCCACTC
CAAGTACGAGACGAACTGCGGCCCGGTGGAGATGCGCGGCCAGCCGGTGTGCGAGGCACGCTCGATGACGATCCACGAGAGCCAGTCGCGC
TTTGCCGAGGTTGTGATTGGCCACTCCAGCGCCTTCTTGGAGTTCCTCGTTCCACTGCTGAAGGAATACCTCGGTGATCAGCCCGCATTCT
CTCGGGAGAACGTGCGGCTGATGAACCAGACGGTGAAGCCTGGCTTCATCCGGATCCGGGCGGATGAGGTGTGCTACCCGCTGCACATCTT
GCTGCGCTACGAGATAGAGCGTGCACTCATCGAGGGCACGATGGAGGCAGAAGACATCCCTCGCGTGTGGAACGAGAAGATGAAGGCATAC
CTGGGCCTGGAGACGGAGGGCCGCGACGAGATTGGCTGCCTGCAGGACATTCACTGGTCGATGGGCGCCTTTGGCTACTTCCCGACGTACT
CGCTTGGCTCCATGTTCGCGGCGCAGCTGATGGCGACGATCAAGAATGAGCTCGGTGAGGATACAGTGGACAAGTGCATCCGCACTGGCCA
GATGGAGCCGATCTTTGAGAAGCAGAGGGAGAAGATCTGGAGCCAGGGATGCCTCTACAACACGGAAGACCTGATTGTCAAGGCGACCGGC
GAAGCGCTGAACCCCAAGTACTTTCGCGAGTACCTGGAACGCCGCTACCTGCGCCAGGAGGACGCTAGCATGGCCTCTTCTCGCTCTGCTC
CCCGCAAGGCTTCCCACGCGCACAAGTCGCACCGCAAGCCGAAGCGCTCGTGGAACGTGTACGTGGGCCGCTCGCTGAAGGCGATCAACGC
CCAGATGTCGATGTCGCACCGCACGGATATCAGCGCCTCCGCTGAGCCGCACAAGGCGGCCGTTGACGTCGGCCCGCTGAGCGTTGGCCCG
CAGAGCGTCGGCCCGCTGAGCGTTGGCCCGCAGGCGGTTGGCCCGCTGAGCGTTGGCCCGCAGAGCGTCGGCCCGCTGAGCGTTGGCCCGC
AGGCGGTTGGCCCGCTGAGCGTTGGCCCGCAGAGCGTTGGCCCGCTGAGCGTTGGCCCGCTGAGCGTTGGCCCGCAGAGCGTTGGCCCGCT
GAGCGTTGGCAGCCAGAGCGTCGGCCCGCTGAGCGTTGGTCCGCAGAGCGTCGGCCCGCTGAGCGTTGGCCCGCAGGCGGTTGGCCCGCTG
AGCGTTGGCCCGCAGAGCGTCGGCCCGCTGAGCGTTGGCCCGCAGGCGGTTGGCCCGCTGAGCGTTGGCCCGCAGAGCGTTGGCCCGCTGA
GCGTTGGCCCGCAGAGCGTTGGCCCGCTGAGCGTTGGCAGCCAGAGCGTCGGCCCGCTGAGCGTTGGTCCGCAGAGCGTCGGCCCGCTGAG
CGTTGGCCCGCAGAGCGTCGGCCCGCTGAGCGTTGGCCCGCAGAGCGTCGGCCCGCTGAGCGTTGGTCCGCAGAGCGTTGGCCCGCTGAGC
GTTGGCCCGCAGAGCGTTGACGTTAGCCCGGTGAGC
SEQ ID NO: 14 Amino Acid Sequence of the 8XHA Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKTSMQAYTQLEKLCQKVYRLAHLLSLGAWD
SKTMMPSKGAAARGAALGELYGLIAEMITSPSTKALLDEAETAKAELTTVQQANLRELRRMYTSQAALPTEFSVLKTKLSSTTPLIWVKCR
SNNDFATFLPALKEMIALARREAQYRSTATGKPLYEALFNQYESGMTLETLEKILLDVKSWLPELLQKILAAQRDAGLEVVAPEAPFPKDK
QEALSRHLMEVWGFDFESGRLDVSEHPFMGMVKEDSRITTAYDLQDFTKGLFATIHETGHSKYETNCGPVEMRGQPVCEARSMTIHESQSR
FAEVVIGHSSAFLEFLVPLLKEYLGDQPAFSRENVRLMNQTVKPGFIRIRADEVCYPLHILLRYEIERALIEGTMEAEDIPRVWNEKMKAY
LGLETEGRDEIGCLQDIHWSMGAFGYFPTYSLGSMFAAQLMATIKNELGEDTVDKCIRTGQMEPIFEKQREKIWSQGCLYNTEDLIVKATG
EALNPKYFREYLERRYLRQEDASMASSRSAPRKASHAHKSHRKPKRSWNVYVGRSLKAINAQMSMSHRTDISASAEPHKAAVDVGPLSVGP
QSVGPLSVGPQAVGPLSVGPQSVGPLSVGPQAVGPLSVGPQSVGPLSVGPLSVGPQSVGPLSVGSQSVGPLSVGPQSVGPLSVGPQAVGPL
SVGPQSVGPLSVGPQAVGPLSVGPQSVGPLSVGPQSVGPLSVGSQSVGPLSVGPQSVGPLSVGPQSVGPLSVGPQSVGPLSVGPQSVGPLS
VGPQSVDVSPVS
8NHA (8E mtHSP70₅₀₉₋₆₆₀ + NH₁₋₃₁₄ + H2B₁₋₄₆ + A2₂₃₋₂₃₆)
8E (1-459) + NH(460-1407) + H2B(1408-1557) + A2(1558-2199)
MW = 76,740

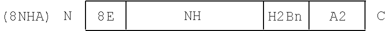

SEQ ID NO: 15 Polynucleotide encoding 8NHA Fusion Polypeptide (underline = linker)
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGACTAGTATGCCGCGCAAGATTATTCTCGATTGTGATCCCGGGATCGATGATGCCGTGGCCATCTTTCTCGCCCACGGCAACCCGGAG
GTCGAGCTGCTGGCCATTACGACGGTGGTGGGCAACCAGACCCTGGAGAAGGTGACCCGGAACGCGCGGCTGGTAGCTGACGTAGCCGGCA
TCGTTGGTGTGCCCGTCGCGGCTGGTTGCACCAAGCCCCTCGTGCGCGGTGTGCGGAATGCCTCTCAGATTCATGGCGAAACCGGCATGGG
TAACGTCTCCTACCCACCAGAGTTCAAGACAAAGTTGGACGGCCGTCATGCAGTGCAGCTGATCATCGACCTTATCATGTCGCACGAGCCG
AAGACGATCACGCTTGTGCCTACGGGTGGCCTGACGAACATTGCGATGGCTGTCCGTCTTGAGCCGCGCATCGTGGACCGTGTGAAGGAGG
TGGTTCTGATGGGTGGCGGCTACCATACTGGTAATGCGTCCCCTGTAGCGGAGTTCAACGTCTTCGTCGACCCGGAGGCGGCGCACATTGT
GTTCAACGAGAGCTGGAACGTAACGATGGTGGGGCTGGACCTAACGCACCAGGCACTCGCCACGCCGGCGGTCCAGAAGCGAGTGAAGGAG
GTGGGCACGAAGCCGGCTGCCTTCATGCTGCAGATTTTGGACTTTTACACGAAGGTGTACGAAAAGGAGCGCAACACGTACGCGACGGTGC
ACGATCCCTGCGCTGTGGCGTACGTGATTGACCCCACCGTGATGACGACGGAGCAAGTGCCAGTGGACATCGAGCTCAATGGGGCACTGAC
GACTGGGATGACGGTCGCGGACTTCCGCTACCCACGGCCAAAGCACTGCCACACGCAGGTGGCTGTGAAGCTGGACTTCGACAAGTTTTGG
TGCCTCGTGATTGACGCACTCAAGCGCATCGGCGATCCTCAAGCTAGCATGGCCTCTTCTCGCTCTGCTCCCCGCAAGGCTTCCCACGCGC
ACAAGTCGCACCGCAAGCCGAAGCGCTCGTGGAACGTGTACGTGGGCCGCTCGCTGAAGGCGATCAACGCCCAGATGTCGATGTCGCACCG
CACGGATATCAGCGCCTCCGCTGAGCCGCACAAGGCGGCCGTTGACGTCGGCCCGCTGAGCGTTGGCCCGCAGAGCGTCGGCCCGCTGAGC
GTTGGCCCGCAGGCGGTTGGCCCGCTGAGCGTTGGCCCGCAGAGCGTCGGCCCGCTGAGCGTTGGCCCGCAGGCGGTTGGCCCGCTGAGCG
TTGGCCCGCAGAGCGTTGGCCCGCTGAGCGTTGGCCCGCTGAGCGTTGGCCCGCAGAGCGTTGGCCCGCTGAGCGTTGGCAGCCAGAGCGT
CGGCCCGCTGAGCGTTGGTCCGCAGAGCGTCGGCCCGCTGAGCGTTGGCCCGCAGGCGGTTGGCCCGCTGAGCGTTGGCCCGCAGAGCGTC
GGCCCGCTGAGCGTTGGCCCGCAGGCGGTTGGCCCGCTGAGCGTTGGCCCGCAGAGCGTTGGCCCGCTGAGCGTTGGCCCGCAGAGCGTTG
GCCCGCTGAGCGTTGGCAGCCAGAGCGTCGGCCCGCTGAGCGTTGGTCCGCAGAGCGTCGGCCCGCTGAGCGTTGGCCCGCAGAGCGTCGG
CCCGCTGAGCGTTGGCCCGCAGAGCGTCGGCCCGCTGAGCGTTGGTCCGCAGAGCGTTGGCCCGCTGAGCGTTGGCCCGCAGAGCGTTGAC
GTTAGCCCGGTGAGC
SEQ ID NO: 16 Amino Acid Sequence of the 8NHA Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENPNVA
KDDLAAATDKLQKAVMECGRTEYQQAAAANSGSTSNSGEQQQQQGQGEQQQQQNSEEKKTSMPRKIILDCDPGIDDAVAIFLAHGNPEVELLAI
TTVVGNQTLEKVTRNARLVADVAGIVGVPVAAGCTKPLVRGVRNASQIHGETGMGNVSYPPEFKTKLDGRHAVQLIIDLIMSHEPKTITLV
PTGGLTNIAMAVRLEPRIVDRVKEVVLMGGGYHTGNASPVAEFNVFVDPEAAHIVFNESWNVTMVGLDLTHQALATPAVQKRVKEVGTKPA
AFMLQILDFYTKVYEKERNTYATVHDPCAVAYVIDPTVMTTEQVPVDIELNGALTTGMTVADFRYPRPKHCHTQVAVKLDFDKFWCLVIDA
LKRIGDPQASMASSRSAPRKASHAHKSHRKPKRSWNVYVGRSLKAINAQMSMSHRTDISASAEPHKAAVDVGPLSVGPQSVGPLSVGPQAV
GPLSVGPQSVGPLSVGPQAVGPLSVGPQSVGPLSVGPLSVGPQSVGPLSVGSQSVGPLSVGPQSVGPLSVGPQAVGPLSVGPQSVGPLSVG
PQAVGPLSVGPQSVGPLSVGPQSVGPLSVGSQSVGPLSVGPQSVGPLSVGPQSVGPLSVGPQSVGPLSVGPQSVGPLSVGPQSVDVSPVS
8CHA (8E mtHSP70₅₀₉₋₆₆₀ + CPB₁₅₄₋₄₄₃ + H2BN₁₋₄₆ + A2₂₃₋₂₃₆)
8E(1-459) + CPB(460-1335) + H2BN(1336-1485) + A2(1486-2127)
MW = 73,633 Daltons

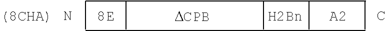

SEQ ID NO: 17 Polynucleotide encoding the 8CHA Fusion Polypeptide (underline = linker)
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGACTAGTTCGGCGGTCGGCAACATCGAGTCGCAGTGGGCCCGTGCCGGCCACGGCTTGGTGAGCCTGTCGGAGCAGCAGCTGGTGAGC
TGCGATGACAAAGACAATGGCTGCAACGGCGGGCTGATGCTGCAGGCGTTCGAGTGGCTGCTGCGACACATGTACGGGATCGTGTTCACGG
AGAAGAGCTACCCCTACACGTCCGGCAACGGTGATGTGGCCGAGTGCTTGAACAGCAGTAAACTCGTTCCCGGCGCGCAAATCGACGGCTA
CGTGATGATCCCGAGCAACGAAACGGTTATGGCTGCGTGGCTTGCGGAGAATGGCCCCATCGCGATTGCGGTCGACGCCAGCTCCTTCATG
TCTTACCAGAGCGGCGTGCTGACCAGCTGCGCTGGCGATGCACTGAACCACGGCGTGCTGCTCGTCGGGTACAACAAGACCGGTGGGGTTC
CGTACTGGGTGATCAAGAACTCGTGGGGTGAGGACTGGGGCGAGAAGGGCTACGTGCGCGTGGTCATGGGGCTGAACGCGTGCCTGCTCAG
TGAATACCCCGTGTCCGCGCATGTGCCGCGGAGTCTCACCCCTGGCCCGGGCACGGAGAGCGAGGAGCGCGCCCCTAAACGGGTGACGGTG
GAGCAGATGATGTGCACCGATATGTACTGCAGGGAGGGGTGCAAGAAGAGTCTTCTCACCGCGAACGTGTGCTACAAGAACGGGGGAGGCG
GCTCCTCTATGACGAAGTGCGGTCCGCAGAAGGTGCTGATGTGCTCGTACTCGAACCCTCATTGCTTTGGTCCTGGGCTGTGCCTCGAGAC
TCCTGATGGCAAGTGCGCGCCGTACTTCTTGGGCTCGATCATGAACACCTGCCAGTACACGGCTAGCATGGCCTCTTCTCGCTCTGCTCCC
CGCAAGGCTTCCCACGCGCACAAGTCGCACCGCAAGCCGAAGCGCTCGTGGAACGTGTACGTGGGCCGCTCGCTGAAGGCGATCAACGCCC
AGATGTCGATGTCGCACCGCACGGATATCAGCGCCTCCGCTGAGCCGCACAAGGCGGCCGTTGACGTCGGCCCGCTGAGCGTTGGCCCGCA
GAGCGTCGGCCCGCTGAGCGTTGGCCCGCAGGCGGTTGGCCCGCTGAGCGTTGGCCCGCAGAGCGTCGGCCCGCTGAGCGTTGGCCCGCAG
GCGGTTGGCCCGCTGAGCGTTGGCCCGCAGAGCGTTGGCCCGCTGAGCGTTGGCCCGCTGAGCGTTGGCCCGCAGAGCGTTGGCCCGCTGA
GCGTTGGCAGCCAGAGCGTCGGCCCGCTGAGCGTTGGTCCGCAGAGCGTCGGCCCGCTGAGCGTTGGCCCGCAGGCGGTTGGCCCGCTGAG
CGTTGGCCCGCAGAGCGTCGGCCCGCTGAGCGTTGGCCCGCAGGCGGTTGGCCCGCTGAGCGTTGGCCCGCAGAGCGTTGGCCCGCTGAGC
GTTGGCCCGCAGAGCGTTGGCCCGCTGAGCGTTGGCAGCCAGAGCGTCGGCCCGCTGAGCGTTGGTCCGCAGAGCGTCGGCCCGCTGAGCG
TTGGCCCGCAGAGCGTCGGCCCGCTGAGCGTTGGCCCGCAGAGCGTCGGCCCGCTGAGCGTTGGTCCGCAGAGCGTTGGCCCGCTGAGCGT
TGGCCCGCAGAGCGTTGACGTTAGCCCGGTGAGC
SEQ ID NO: 18 Amino Acid Sequence of the 8CHA Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENPNVA
KDDLAAATDKLQKAVMECGRTEYQQAAAANSGSTSNSGEQQQQQGQGEQQQQQNSEEKKTSSAVGNIESQWARAGHGLVSLSEQQLVSCDDKDN
GCNGGLMLQAFEWLLRHMYGIVFTEKSYPYTSGNGDVAECLNSSKLVPGAQIDGYVMIPSNETVMAAWLAENGPIAIAVDASSFMSYQSGV
LTSCAGDALNHGVLLVGYNKTGGVPYWVIKNSWGEDWGEKGYVRVVMGLNACLLSEYPVSAHVPRSLTPGPGGCKKSLLTANVCYKNGGGG
SSMTKCGPQKVLMCSYSNTESEERAPKRVTVEQMMCTDMYCREPHCFGPGLCLETPDGKCAPYFLGSIMNTCQYTASMASSRSAPRKASHA
HKSHRKPKRSWNVYVGRSLKAINAQMSMSHRTDISASAEPHKAAVDVGPLSVGPQSVGPLSVGPQAVGPLSVGPQSVGPLSVGPQAVGPLS
VGPQSVGPLSVGPLSVGPQSVGPLSVGSQSVGPLSVGPQSVGPLSVGPQAVGPLSVGPQSVGPLSVGPQAVGPLSVGPQSVGPLSVGPQSV
GPLSVGSQSVGPLSVGPQSVGPLSVGPQSVGPLSVGPQSVGPLSVGPQSVGPLSVGPQSVDVSPVS
8NCA (8E mtHSP70₅₀₉₋₆₆₀ + NH₁₋₃₁₄ + CPB₁₅₄₋₄₄₃ + A2₂₃₋₂₃₆)

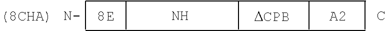

8E (1 . . . 459) + NH (460 . . . 1407) + CPB (1408 . . . 2283) + A2(2284 . . . 2931)
MW = 102,641 Daltons
SEQ ID NO: 19 Polynucleotide encoding the 8NCA Fusion Polypeptide (underline = linker)
ATGAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGACTAGTATGCCGCGCAAGATTATTCTCGATTGTGATCCCGGGATCGATGATGCCGTGGCCATCTTTCTCGCCCACGGCAACCCGGAG
GTCGAGCTGCTGGCCATTACGACGGTGGTGGGCAACCAGACCCTGGAGAAGGTGACCCGGAACGCGCGGCTGGTAGCTGACGTAGCCGGCA
TCGTTGGTGTGCCCGTCGCGGCTGGTTGCACCAAGCCCCTCGTGCGCGGTGTGCGGAATGCCTCTCAGATTCATGGCGAAACCGGCATGGG
TAACGTCTCCTACCCACCAGAGTTCAAGACAAAGTTGGACGGCCGTCATGCAGTGCAGCTGATCATCGACCTTATCATGTCGCACGAGCCG
AAGACGATCACGCTTGTGCCTACGGGTGGCCTGACGAACATTGCGATGGCTGTCCGTCTTGAGCCGCGCATCGTGGACCGTGTGAAGGAGG
TGGTTCTGATGGGTGGCGGCTACCATACTGGTAATGCGTCCCCTGTAGCGGAGTTCAACGTCTTCGTCGACCCGGAGGCGGCGCACATTGT
GTTCAACGAGAGCTGGAACGTAACGATGGTGGGGCTGGACCTAACGCACCAGGCACTCGCCACGCCGGCGGTCCAGAAGCGAGTGAAGGAG
GTGGGCACGAAGCCGGCTGCCTTCATGCTGCAGATTTTGGACTTTTACACGAAGGTGTACGAAAAGGAGCGCAACACGTACGCGACGGTGC
ACGATCCCTGCGCTGTGGCGTACGTGATTGACCCCACCGTGATGACGACGGAGCAAGTGCCAGTGGACATCGAGCTCAATGGGGCACTGAC
GACTGGGATGACGGTCGCGGACTTCCGCTACCCACGGCCAAAGCACTGCCACACGCAGGTGGCTGTGAAGCTGGACTTCGACAAGTTTTGG
TGCCTCGTGATTGACGCACTCAAGCGCATCGGCGATCCTCAAGCTAGCTCGGCGGTCGGCAACATCGAGTCGCAGTGGGCCCGTGCCGGCC
ACGGCTTGGTGAGCCTGTCGGAGCAGCAGCTGGTGAGCTGCGATGACAAAGACAATGGCTGCAACGGCGGGCTGATGCTGCAGGCGTTCGA
GTGGCTGCTGCGACACATGTACGGGATCGTGTTCACGGAGAAGAGCTACCCCTACACGTCCGGCAACGGTGATGTGGCCGAGTGCTTGAAC
AGCAGTAAACTCGTTCCCGGCGCGCAAATCGACGGCTACGTGATGATCCCGAGCAACGAAACGGTTATGGCTGCGTGGCTTGCGGAGAATG
GCCCCATCGCGATTGCGGTCGACGCCAGCTCCTTCATGTCTTACCAGAGCGGCGTGCTGACCAGCTGCGCTGGCGATGCACTGAACCACGG
CGTGCTGCTCGTCGGGTACAACAAGACCGGTGGGGTTCCGTACTGGGTGATCAAGAACTCGTGGGGTGAGGACTGGGGCGAGAAGGGCTAC
GTGCGCGTGGTCATGGGGCTGAACGCGTGCCTGCTCAGTGAATACCCCGTGTCCGCGCATGTGCCGCGGAGTCTCACCCCTGGCCCGGGCA
CGGAGAGCGAGGAGCGCGCCCCTAAACGGGTGACGGTGGAGCAGATGATGTGCACCGATATGTACTGCAGGGAGGGGTGCAAGAAGAGTCT
TCTCACCGCGAACGTGTGCTACAAGAACGGGGGAGGCGGCTCCTCTATGACGAAGTGCGGTCCGCAGAAGGTGCTGATGTGCTCGTACTCG
AACCCTCATTGCTTTGGTCCTGGGCTGTGCCTCGAGACTCCTGATGGCAAGTGCGCGCCGTACTTCTTGGGCTCGATCATGAACACCTGCC
AGTACACGGATATCAGCGCCTCCGCTGAGCCGCACAAGGCGGCCGTTGACGTCGGCCCGCTGAGCGTTGGCCCGCAGAGCGTCGGCCCGCT
GAGCGTTGGCCCGCAGGCGGTTGGCCCGCTGAGCGTTGGCCCGCAGAGCGTCGGCCCGCTGAGCGTTGGCCCGCAGGCGGTTGGCCCGCTG
AGCGTTGGCCCGCAGAGCGTTGGCCCGCTGAGCGTTGGCCCGCTGAGCGTTGGCCCGCAGAGCGTTGGCCCGCTGAGCGTTGGCAGCCAGA
GCGTCGGCCCGCTGAGCGTTGGTCCGCAGAGCGTCGGCCCGCTGAGCGTTGGCCCGCAGGCGGTTGGCCCGCTGAGCGTTGGCCCGCAGAG
CGTCGGCCCGCTGAGCGTTGGCCCGCAGGCGGTTGGCCCGCTGAGCGTTGGCCCGCAGAGCGTTGGCCCGCTGAGCGTTGGCCCGCAGAGC
GTTGGCCCGCTGAGCGTTGGCAGCCAGAGCGTCGGCCCGCTGAGCGTTGGTCCGCAGAGCGTCGGCCCGCTGAGCGTTGGCCCGCAGAGCG
TCGGCCCGCTGAGCGTTGGCCCGCAGAGCGTCGGCCCGCTGAGCGTTGGTCCGCAGAGCGTTGGCCCGCTGAGCGTTGGCCCGCAGAGCGT
TGACGTTAGCCCGGTGAGC
SEQ ID NO: 20 Amino Acid Sequence of the 8NCA Fusion Polypeptide (double underline =
linker)
MKDKATGKTQNITITANGGLSKEQIEQMIRDSEQHAEADRVKRELVEVRNNAETQLTTAERQLGEWKYVSDAEKENVKTLVAELRKAMENPNVA
KDDLAAATDKLQKAVMECGRTEYQQAAAANSGSTSNSGEQQQQQGQGEQQQQQNSEEKKTSMPRKIILDCDPGIDDAVAIFLAHGNPEVELLAI
TTVVGNQTLEKVTRNARLVADVAGIVGVPVAAGCTKPLVRGVRNASQIHGETGMGNVSYPPEFKTKLDGRHAVQLIIDLIMSHEPKTITLV
PTGGLTNIAMAVRLEPRIVDRVKEVVLMGGGYHTGNASPVAEFNVFVDPEAAHIVFNESWNVTMVGLDLTHQALATPAVQKRVKEVGTKPA
AFMLQILDFYTKVYEKERNTYATVHDPCAVAYVIDPTVMTTEQVPVDIELNGALTTGMTVADFRYPRPKHCHTQVAVKLDFDKFWCLVIDA
LKRIGDPQASSAVGNIESQWARAGHGLVSLSEQQLVSCDDKDNGCNGGLMLQAFEWLLRHMYGIVFTEKSYPYTSGNGDVAECLNSSKLVP
GAQIDGYVMIPSNETVMAAWLAENGPIAIAVDASSFMSYQSGVLTSCAGDALNHGVLLVGYNKTGGVPYWVIKNSWGEDWGEKGYVRVVMG
LNACLLSEYPVSAHVPRSLTPGPGTESEERAPKRVTVEQMMCTDMYCREGCKKSLLTANVCYKNGGGGSSMTKCGPQKVLMCSYSNPHCFG
PGLCLETPDGKCAPYFLGSIMNTCQYTDISASAEPHKAAVDVGPLSVGPQSVGPLSVGPQAVGPLSVGPQSVGPLSVGPQAVGPLSVGPQS
VGPLSVGPLSVGPQSVGPLSVGSQSVGPLSVGPQSVGPLSVGPQAVGPLSVGPQSVGPLSVGPQAVGPLSVGPQSVGPLSVGPQSVGPLSV
GSQSVGPLSVGPQSVGPLSVGPQSVGPLSVGPQSVGPLSVGPQSVGPLSVGPQSVDVSPVS
SEQ ID NO: 21 Amino Acid Sequence of the mitochondrial HSP70 polypeptide (8E) from
Leishmania infantum or donovani
KDKATGKTQNITITANGGLSKEQIEQMIRDSEQHAEADRVKRELVEVRNNAETQLTTAERQLGEWKYVSDAEKENVKTLVAELRKAMENPN
VAKDDLAAATDKLQKAVMECGRTEYQQAAAANSGSTSNSGEQQQQQGQGEQQQQQNSEEKK
SEQ ID NO: 22 Amino Acid Sequence of the mitochondrial HSP70 polypeptide (8E) from
Leishmania major
KDKATGKTQNITITANGGLSKEQIEQMIRDSEQHAEADRVKRELVEVRNNAETQLTTAERQLGEWKYVSDAEKENVKTLVAELRKAMENPN
VAKDDLAAATDKLQKAVMECGRTEYQQAAAANSGSTSNSGEQQQQQSQGEQQQQQNSEEKK
SEQ ID NO: 23 Amino Acid Sequence of the mitochondrial HSP70 polypeptide (8E) from
Leishmania mexicana
KDKATGKTQNITITANGGLSKEQIEQMIRDSEQHAEADRVKRELVEVRNNAETQLTTAERQLSEWKYVSDAEKENVRTLVAELRKAMENPN
VAKDDLSAATDKLQKAVMECGRTEYQQAAAANSGSTSNSGEQQQQQQQSQGEQQQQQQQQQQAEER
SEQ ID NO: 24 Amino Acid Sequence of the mitochondrial HSP70 polypeptide (8E) from
Leishmania braziliensis
KDKATGKTQNITITAHGGLSKEQIEQMVRDSEQHAEADRVKRELVEARNNAETQLTTAERQLGEWKYVSDAEKENVKTHVAELRKAMENPN
VAKDDLAAATDKLQKAVMECGRTEYQQAAAANSGSSSNSGEQQQQQQQQGDQQQQQSSEKN
SEQ ID NO: 25 Amino Acid Sequence of the carboxypeptidase polypeptide (CxP) from
Leishmania donovani
MQAYTQLEKLCQKVYRLAHLLSLGAWDSKTMMPSKGAAARGAALGELYGLIAEMITSPSTKALLDEAETAKAELTTVQQANLRELRRMYTS
QAALPTEFSVLKTKLSSTTPLIWVKCRSNNDFATFLPALKEMIALARREAQYRSTATGKPLYEALFNQYESGMTLETLEKILLDVKSWLPE
LLQKILAAQRDAGLEVVAPEAPFPKDKQEALSRHLMEVWGFDFESGRLDVSEHPFMGMVKEDSRITTAYDLQDFTKGLFATIHETGHSKYE
TNCGPVEMRGQPVCEARSMTIHESQSRFAEVVIGHSSAFLEFLVPLLKEYLGDQPAFSRENVRLMNQTVKPGFIRIRADEVCYPLHILLRY
EIERALIEGTMEAEDIPRVWNEKMKAYLGLETEGRDEIGCLQDIHWSMGAFGYFPTYSLGSMFAAQLMATIKNELGEDTVDKCIRTGQMEP
IFEKQREKIWSQGCLYNTEDLIVKATGEALNPKYFREYLERRYLRQED
SEQ ID NO: 26 Amino Acid Sequence of the carboxypeptidase polypeptide (CxP) from
Leishmania infantum
MQAYTQLEKLCQKVYRLAHLLSLGAWDSKTMMPSKGAAARGAALGELYGLIAEMITSPSTKALLDEAEAAKAELTTVQQANLRELRRMYTS
QAALPTEFSVLKTKLSSTTPLIWAKCRSNNDFATFLPALKEMIALARREAQYRSTATGKPLYEALFNQYESGMTLETLEKILLDVKSWLPE
LLQKILAAQRDAGLEVVAPEAPFPKDKQEALSRHLMEVWGFDFESGRLDVSEHPFMGMVKEDSRITTAYDLQDFTKGLFATIHETGHSKYE
TNCGPVEMRGQPVCEARSMTIHESQSRFAEVVIGHSSAFLEFLVPLLKEYLGDQPAFSRENVRLMNQTVKPGFIRIRADEVCYPLHILLRY
EIERALIEGTMEAEDIPRVWNEKMKAYLGLETEGRDEIGCLQDINWSMGAFGYFPTYSLGSMFAAQLMATIKNELGEDTVDKCIRTGQMEP
IFEKQREKIWSQGCLYNTEDLIVKATGEALNPKYFREYLERRYLRQED
SEQ ID NO: 27 Amino Acid Sequence of the carboxypeptidase polypeptide (CxP) from
Leishmania major
MQAYTQLEKLCHKVHRLTHLLSLGAWDAKTMMPSKGAAARGAALGELHGLITEMITSPSTKALLDEAETAKAELTTVQQANLRELRRIYAS
QAALPTELRVLKTKLSATTPLIWAKCRSNNDFATFLPALKEMIALARREAQYRSAATGKPLYEALFNQYESGMTLETLEKILLDVKSWLPE
LLQKILAAQRDAGLEVVAPEAPFPKDKQEALSRHLMEVWGFDFESGRLDVSEHPFTGMVKEDSRITTAYDLQDFAKGLFATIHETGHSKYE
TNCGPMEMRGQPVCEARSMTIHESQSRFAEVVIGHSSAFLEFLTPLLKEYFGDQPAFSLENVRLMNQTVKPGFIRIRADEVCYPLHILLRY
EIERALIEGTMEAEDIPRVWNEKMKAYLGLETEGRDEIGCLQDIHWSMGAFGYFPTYSLGSMFAAQLMVTIKNELGEDTVDKCIRTGQMEP
IFEKQREKIWSQGCLYDTEDLILKATGEALNPKHFREYLERRYLRQEG
SEQ ID NO: 28 Amino Acid Sequence of the carboxypeptidase polypeptide (CxP) from
Leishmania mexicana
MQAYSQLEKLCQKVYRLEHLLSLGAWDAKTMMPSKGAAARGAALGELYGLIAEMITSPSTKTLLDEAETAKAELTTVQQANLRELRRMYTS
QAALPTEFSVLKAKLSSTTPLIWAKCRSNNDFVTFLPALKEMIALARREAQYRSTATGKPLYEALFNQYESGMTLETLEKNLLDVKSWLPE
LLQKILAAQKDAGREAVAPEAPFPKDKQEALSRHLMKVWGFDFESGRLDVSEHPFMGMVKEDSRITTAYDLQDFTKGLFATIHETGHSKYE
TNCGPMEMRGQPVCEARSMTIHESQSRFAEVVIGHSSAFLEFLVPLLKEYLGDQPTLSLENVRLMNQTVKPGFIRIRADEVCYPLHILLRY
EIERALIEGTMEAEDIPRVWNEKMKAYLGLETEGRDEIGCLQDIHWPMGAFGYFPTYSLGSMFAVQLMATIKKELGEDTVDKCIRTGQMEP
IFQKQREKIWSQGCLYNTEDLIVKATGETLNPKHFREYLERRYLRQED
SEQ ID NO: 29 Amino Acid Sequence of the carboxypeptidase polypeptide (CxP) from
Leishmania braziliensis
MQAYKQLEQLSQKLHNLSHFLYLGKWDSETMMPSKGSAARGAAIGELHGLIAELMTAPSTKTLLDEAEGVKTELTKTQQANLREFRRMYSA
QAALPNDFSMLKARLSTTVPLIWAECRRNNDFATFVPTLKEVITVARKEAQYRSAATGKPLYEALFNQYECGMTLETVDSIFSDVKSWLPE
LLQKILTLQKAEGLEARAPEAPFPKDKQDALGRHLMKVWGFDFESGRLDVSAHPFTGMVKEDSRITTNYDLEDFTKALFATIHETGHSKYE
TNCGPMDMRGQPVCNARSLMIHESQSRFAEVVIGRSSAFPEFLAPLLKEHLGEQPAFSLENVRLMSQRVRPGFIRIFADEVCYPLHVLLRY
EIERALIEGTMEVEDIPRVWNEKMKAYLGLETEGRDDIGCLQDTHWAMGAFGYFPTYTLGTMFAVQLMYTIKKELGESTVDKCIRTGQMEP
IFAKQKEKIWDQGCLYETEELMIKATGETLNPKYFREYLERRYLRHED
SEQ ID NO: 30 Amino Acid Sequence of a carboxy terminus fragment of cysteine
polypeptidease B polypeptide (CPB) from Leishmania infantum
SAVGNIESQWARAGHGLVSLSEQQLVSCDDKDNGCNGGLMLQAFEWLLRHMYGIVFTEKSYPYTSGNGDVAECLNSSKLVPGAQIDGYVMI
PSNETVMAAWLAENGPIAIAVDASSFMSYQSGVLTSCAGDALNHGVLLVGYNKTGGVPYWVIKNSWGEDWGEKGYVRVVMGLNACLLSEYP
VSAHVPRSLTPGPGTESEERAPKRVTVEQMMCTDMYCREGCKKSLLTANVCYKNGGGGSSMTKCGPQKVLMCSYSNPHCFGPGLCLETPDG
KCAPYFLGSIMNTCQYT
SEQ ID NO: 31 Amino Acid Sequence of an amino terminus fragment of histone H2BN
polypeptide (H2BN) from Leishmania infantum
MASSRSAPRKASHAHKSHRKPKRSWNVYVGRSLKAINAQMSMSHRT
SEQ ID NO: 32 Amino Acid Sequence of a mature A2 polypeptide (A) from Leishmania
donovani
SASAEPHKAAVDVGPLSVGPQSVGPLSVGPQAVGPLSVGPQSVGPLSVGPQAVGPLSVGPQSVGPLSVGPLSVGPQSVGPLSVGSQSVGPL
SVGPQSVGPLSVGPQAVGPLSVGPQSVGPLSVGPQAVGPLSVGPQSVGPLSVGPQSVGPLSVGSQSVGPLSVGPQSVGPLSVGPQSVGPLS
VGPQSVGPLSVGPQSVGPLSVGPQSVDVSPVS
SEQ ID NO: 33 Amino Acid Sequence of a full length p21 antigen polypeptide (p21) from
Leishmania infantum
SIIKEDDAVGCYMTVTLVDDTKVEGTIFTYNPKEGIIVLLSLRDDQTNMKLIRTPYIKEFSISHAEEGTHLPPALDSFNELPSMHAGRDKS
IFKHASTQLKNAEANREKHFNSVTTDTPIATLDAYLKLLRLYPFIEWNSDEGVIQVSDTVIVVGDPDWRTPKAMLVDGAPEKDRPLVDRLQ
VALGNGKK
SEQ ID NO: 34 Amino Acid Sequence of a full length thiol specific antioxidant polypeptide
(TSA) from Leishmania major
MQAYTQLEKLCQKVYRLAHLLSLGAWDSKTMMPSKGAAARGAALGELYGLIAEMITSPSTKALLDEAETAKAELTTVQQANLRELRRMYTS
QAALPTEFSVLKTKLSSTTPLIWVKCRSNNDFATFLPALKEMIALARREAQYRSTATGKPLYEALFNQYESGMTLETLEKILLDVKSWLPE
LLQKIL
SEQ ID NO: 35 Amino Acid Sequence of a putative eukaryotic initiation factor 4a polypeptide
(Leif) from Leishmania major
MAQNDKIAPQDQDSFLDDQPGVRPIPSFDDMPLHQNLLRGIYSYGFEKPSSIQQRAIAPFTRGGDIIAQAQSGTGKTGAFSIGLLQRLDFR
HNLIQGLVLSPTRELALQTAEVISRIGEFLSNSSKFCETFVGGTRVQDDLRKLQAGVIVAVGTPGRVSDVIKRGALRTESLRVLVLDEADE
MLSQGFADQIYEIFRFLPKDIQVALFSATMPEEVLELTKKFMRD
SEQ ID NO: 36 Amino Acid Sequence of a full length nonspecific nucleoside hydrolase
polypeptide (NH) from Leishmania infantum or donovani
MPRKIILDCDPGIDDAVAIFLAHGNPEVELLAITTVVGNQTLEKVTRNARLVADVAGIVGVPVAAGCTKPLVRGVRNASQIHGETGMGNVS
YPPEFKTKLDGRHAVQLIIDLIMSHEPKTITLVPTGGLTNIAMAVRLEPRIVDRVKEVVLMGGGYHTGNASPVAEFNVFVDPEAAHIVFNE
SWNVTMVGLDLTHQALATPAVQKRVKEVGTKPAAFMLQILDFYTKVYEKERNTYATVHDPCAVAYVIDPTVMTTEQVPVDIELNGALTTGM
TVADFRYPRPKHCHTQVAVKLDFDKFWCLVIDALKRIGDPQ
SEQ ID NO: 37 Amino Acid Sequence of a full length A2 polypeptide (Afl) from Leishmania
donovani
MKIRSVRPLVVLLVSVAAVLALSASAEPHKAAVDVGPLSVGPQSVGPLSVGPQAVGPLSVGPQSVGPLSVGPQAVGPLSVGPQSVGPLSVG
PLSVGPQSVGPLSVGSQSVGPLSVGPQSVGPLSVGPQAVGPLSVGPQSVGPLSVGPQAVGPLSVGPQSVGPLSVGPQSVGPLSVGSQSVGP
LSVGPQSVGPLSVGPQSVGPLSVGPQSVGPLSVGPQSVGPLSVGPQSVDVSPVS
SEQ ID NO: 38Amino Acid Sequence of Alpha Tubulin (aT) from Leishmania infantum
MRDAHTRTPTEKKTRSSSLSFFEQTPLNRLLTPLSSFSAMREAICIHIGQAGCQVGNACWELFCLEHGIQPDGSMPSDKCIGVEDDAFNTF
FSETGAGKHVPRCIFLDLEPTVVDEVRTGTYRQLFNPEQLVSGKEDAANNYARGHYTIGKEIVDLALDRIRKLADNCTGLQGFMVFHAVGG
GTGSGLGALLLERLSVDYGKKSKLGYTVYPSPQVSTAVVEPYNCVLSTHSLLEHTDVATMLDNEAIYDLTRRSLDIERPSYTNVNRLIGQV
VSSLTASLRFDGALNVDLTEFQTNLVPYPRIHFVLTSYAPVVSAEKAYHEQLSVADITNSVFEPAGMLTKCDPRHGKYMSCCLMYRGDVVP
KDVNAAIATIKTKRTIQFVDWCPTGFKCGINYQPPTVVPGGDLAKVQRAVCMIANSTAIAEVFARIDHKFDLMYSKRAFVHWYVGEGMEEG
EFSEAREDLAALEKDYEEVGAESADDMGEEDVEEY
SEQ ID NO: 39 Amino Acid Sequence of Malate dehydrogenase (MDH) from Leishmania
infantum
MVNVCVVGAAGGIGQSLSLLLVRQLPYGSTLSLFDVVGAAGVAADLSHVDNAGVQVKFAAGKIGQKRDPALAELAKGVDVFVMVAGVPRKP
GMTRDDLFKINAGIILDLVLTCASSSPKAVFCIVTNPVNSTVVIAAEALKSLGVYDRNRLLGVSLLDGLRATCFINEARKPLVVTQVPVVG
GHSDATIVPLFHQLLGPLPEQATLDKIVKRVQVAGTEVVKAKAGRGSATLSMAEAGARFTLKVVEGLTGTGKPLVYAYVDTDGQHETPFLA
IPVVLGVNGIEKRLPIGPLHSTEETLLKAALPVIKKNIVKGSEFARSHL
8NC: mtHSP70₅₀₉₋₆₆₀ + NH₁₋₃₁₄ + CPB₁₅₄₋₄₄₃
8E (1 . . . 459) + NH (460 . . . 1401) + CPB (1402 . . . 2271)
MW = 82330 Daltons

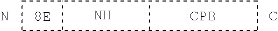

SEQ ID NO: 40 Polynucleotide encoding the 8NC Fusion Polypeptide
atgAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGCCGCGCAAGATTATTCTCGATTGTGATCCCGGGATCGATGATGCCGTGGCCATCTTTCTCGCCCACGGCAACCCGGAGGTCGAG
CTGCTGGCCATTACGACGGTGGTGGGCAACCAGACCCTGGAGAAGGTGACCCGGAACGCGCGGCTGGTAGCTGACGTAGCCGGCATCGTTG
GTGTGCCCGTCGCGGCTGGTTGCACCAAGCCCCTCGTGCGCGGTGTGCGGAATGCCTCTCAGATTCATGGCGAAACCGGCATGGGTAACGT
CTCCTACCCACCAGAGTTCAAGACAAAGTTGGACGGCCGTCATGCAGTGCAGCTGATCATCGACCTTATCATGTCGCACGAGCCGAAGACG
ATCACGCTTGTGCCTACGGGTGGCCTGACGAACATTGCGATGGCTGTCCGTCTTGAGCCGCGCATCGTGGACCGTGTGAAGGAGGTGGTTC
TGATGGGTGGCGGCTACCATACTGGTAATGCGTCCCCTGTAGCGGAGTTCAACGTCTTCGTCGACCCGGAGGCGGCGCACATTGTGTTCAA
CGAGAGCTGGAACGTAACGATGGTGGGGCTGGACCTAACGCACCAGGCACTCGCCACGCCGGCGGTCCAGAAGCGAGTGAAGGAGGTGGGC
ACGAAGCCGGCTGCCTTCATGCTGCAGATTTTGGACTTTTACACGAAGGTGTACGAAAAGGAGCGCAACACGTACGCGACGGTGCACGATC
CCTGCGCTGTGGCGTACGTGATTGACCCCACCGTGATGACGACGGAGCAAGTGCCAGTGGACATCGAGCTCAATGGGGCACTGACGACTGG
GATGACGGTCGCGGACTTCCGCTACCCACGGCCAAAGCACTGCCACACGCAGGTGGCTGTGAAGCTGGACTTCGACAAGTTTTGGTGCCTC
GTGATTGACGCACTCAAGCGCATCGGCGATCCTCAATCGGCGGTCGGCAACATCGAGTCGCAGTGGGCCCGTGCCGGCCACGGCTTGGTGA
GCCTGTCGGAGCAGCAGCTGGTGAGCTGCGATGACAAAGACAATGGCTGCAACGGCGGGCTGATGCTGCAGGCGTTCGAGTGGCTGCTGCG
ACACATGTACGGGATCGTGTTCACGGAGAAGAGCTACCCCTACACGTCCGGCAACGGTGATGTGGCCGAGTGCTTGAACAGCAGTAAACTC
GTTCCCGGCGCGCAAATCGACGGCTACGTGATGATCCCGAGCAACGAAACGGTTATGGCTGCGTGGCTTGCGGAGAATGGCCCCATCGCGA
TTGCGGTCGACGCCAGCTCCTTCATGTCTTACCAGAGCGGCGTGCTGACCAGCTGCGCTGGCGATGCACTGAACCACGGCGTGCTGCTCGT
CGGGTACAACAAGACCGGTGGGGTTCCGTACTGGGTGATCAAGAACTCGTGGGGTGAGGACTGGGGCGAGAAGGGCTACGTGCGCGTGGTC
ATGGGGCTGAACGCGTGCCTGCTCAGTGAATACCCCGTGTCCGCGCATGTGCCGCGGAGTCTCACCCCTGGCCCGGGCACGGAGAGCGAGG
AGCGCGCCCCTAAACGGGTGACGGTGGAGCAGATGATGTGCACCGATATGTACTGCAGGGAGGGGTGCAAGAAGAGTCTTCTCACCGCGAA
CGTGTGCTACAAGAACGGGGGAGGCGGCTCCTCTATGACGAAGTGCGGTCCGCAGAAGGTGCTGATGTGCTCGTACTCGAACCCTCATTGC
TTTGGTCCTGGGCTGTGCCTCGAGACTCCTGATGGCAAGTGCGCGCCGTACTTCTTGGGCTCGATCATGAACACCTGCCAGTACACG
SEQ ID NO: 41 Amino Acid Sequence of the 8NC Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMPRKIILDCDPGIDDAVAIFLAHGNPEVE
LLAITTVVGNQTLEKVTRNARLVADVAGIVGVPVAAGCTKPLVRGVRNASQIHGETGMGNVSYPPEFKTKLDGRHAVQLIIDLIMSHEPKT
ITLVPTGGLTNIAMAVRLEPRIVDRVKEVVLMGGGYHTGNASPVAEFNVFVDPEAAHIVFNESWNVTMVGLDLTHQALATPAVQKRVKEVG
TKPAAFMLQILDFYTKVYEKERNTYATVHDPCAVAYVIDPTVMTTEQVPVDIELNGALTTGMTVADFRYPRPKHCHTQVAVKLDFDKFWCL
VIDALKRIGDPQSAVGNIESQWARAGHGLVSLSEQQLVSCDDKDNGCNGGLMLQAFEWLLRHMYGIVFTEKSYPYTSGNGDVAECLNSSKL
VPGAQIDGYVMIPSNETVMAAWLAENGPIAIAVDASSFMSYQSGVLTSCAGDALNHGVLLVGYNKTGGVPYWVIKNSWGEDWGEKGYVRVV
MGLNACLLSEYPVSAHVPRSLTPGPGTESEERAPKRVTVEQMMCTDMYCREGCKKSLLTANVCYKNGGGGSSMTKCGPQKVLMCSYSNPHC
FGPGLCLETPDGKCAPYFLGSIMNTCQYT
8NCH: mtHSP70₅₀₉₋₆₆₀ + NH₁₋₃₁₄ + CPB₁₅₄₋₄₄₃ + H2B₁₋₁₁₁
8E (1 . . .459) + NH (460 . . . 1401) + CPB (1402 . . . 2271) + H2Bn (2272 . . . 2604)
MW = 94471 Daltons

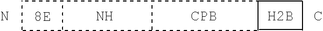

SEQ ID NO: 42 Polynucleotide encoding the 8NCH Fusion Polypeptide
atgAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGCCGCGCAAGATTATTCTCGATTGTGATCCCGGGATCGATGATGCCGTGGCCATCTTTCTCGCCCACGGCAACCCGGAGGTCGAG
CTGCTGGCCATTACGACGGTGGTGGGCAACCAGACCCTGGAGAAGGTGACCCGGAACGCGCGGCTGGTAGCTGACGTAGCCGGCATCGTTG
GTGTGCCCGTCGCGGCTGGTTGCACCAAGCCCCTCGTGCGCGGTGTGCGGAATGCCTCTCAGATTCATGGCGAAACCGGCATGGGTAACGT
CTCCTACCCACCAGAGTTCAAGACAAAGTTGGACGGCCGTCATGCAGTGCAGCTGATCATCGACCTTATCATGTCGCACGAGCCGAAGACG
ATCACGCTTGTGCCTACGGGTGGCCTGACGAACATTGCGATGGCTGTCCGTCTTGAGCCGCGCATCGTGGACCGTGTGAAGGAGGTGGTTC
TGATGGGTGGCGGCTACCATACTGGTAATGCGTCCCCTGTAGCGGAGTTCAACGTCTTCGTCGACCCGGAGGCGGCGCACATTGTGTTCAA
CGAGAGCTGGAACGTAACGATGGTGGGGCTGGACCTAACGCACCAGGCACTCGCCACGCCGGCGGTCCAGAAGCGAGTGAAGGAGGTGGGC
ACGAAGCCGGCTGCCTTCATGCTGCAGATTTTGGACTTTTACACGAAGGTGTACGAAAAGGAGCGCAACACGTACGCGACGGTGCACGATC
CCTGCGCTGTGGCGTACGTGATTGACCCCACCGTGATGACGACGGAGCAAGTGCCAGTGGACATCGAGCTCAATGGGGCACTGACGACTGG
GATGACGGTCGCGGACTTCCGCTACCCACGGCCAAAGCACTGCCACACGCAGGTGGCTGTGAAGCTGGACTTCGACAAGTTTTGGTGCCTC
GTGATTGACGCACTCAAGCGCATCGGCGATCCTCAATCGGCGGTCGGCAACATCGAGTCGCAGTGGGCCCGTGCCGGCCACGGCTTGGTGA
GCCTGTCGGAGCAGCAGCTGGTGAGCTGCGATGACAAAGACAATGGCTGCAACGGCGGGCTGATGCTGCAGGCGTTCGAGTGGCTGCTGCG
ACACATGTACGGGATCGTGTTCACGGAGAAGAGCTACCCCTACACGTCCGGCAACGGTGATGTGGCCGAGTGCTTGAACAGCAGTAAACTC
GTTCCCGGCGCGCAAATCGACGGCTACGTGATGATCCCGAGCAACGAAACGGTTATGGCTGCGTGGCTTGCGGAGAATGGCCCCATCGCGA
TTGCGGTCGACGCCAGCTCCTTCATGTCTTACCAGAGCGGCGTGCTGACCAGCTGCGCTGGCGATGCACTGAACCACGGCGTGCTGCTCGT
CGGGTACAACAAGACCGGTGGGGTTCCGTACTGGGTGATCAAGAACTCGTGGGGTGAGGACTGGGGCGAGAAGGGCTACGTGCGCGTGGTC
ATGGGGCTGAACGCGTGCCTGCTCAGTGAATACCCCGTGTCCGCGCATGTGCCGCGGAGTCTCACCCCTGGCCCGGGCACGGAGAGCGAGG
AGCGCGCCCCTAAACGGGTGACGGTGGAGCAGATGATGTGCACCGATATGTACTGCAGGGAGGGGTGCAAGAAGAGTCTTCTCACCGCGAA
CGTGTGCTACAAGAACGGGGGAGGCGGCTCCTCTATGACGAAGTGCGGTCCGCAGAAGGTGCTGATGTGCTCGTACTCGAACCCTCATTGC
TTTGGTCCTGGGCTGTGCCTCGAGACTCCTGATGGCAAGTGCGCGCCGTACTTCTTGGGCTCGATCATGAACACCTGCCAGTACACGATGG
CCTCTTCTCGCTCTGCTCCCCGCAAGGCTTCCCACGCGCACAAGTCGCACCGCAAGCCGAAGCGCTCGTGGAACGTGTACGTGGGCCGCTC
GCTGAAGGCGATCAACGCCCAGATGTCGATGTCGCACCGCACGATGAGCATCGTGAACTCGTACGTGAACGACGTGATGGAGCGCATCTGC
ATGGAGGCCGCGTCGATCGTTCGCGCGAACAAGAAGCGCACGTTGGGTGCGCGCGAGGTGCAGACGGCGGTGCGCATTGTGCTGCCGGCGG
AGCTCGCGAAGCACGCCATGGCTGAGGGCACGAAGGCCGTGTCGAGCGCGTCGGCT
SEQ ID NO: 43 Amino Acid Sequence of the 8NCH Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMPRKIILDCDPGIDDAVAIFLAHGNPEVE
LLAITTVVGNQTLEKVTRNARLVADVAGIVGVPVAAGCTKPLVRGVRNASQIHGETGMGNVSYPPEFKTKLDGRHAVQLIIDLIMSHEPKT
ITLVPTGGLTNIAMAVRLEPRIVDRVKEVVLMGGGYHTGNASPVAEFNVFVDPEAAHIVFNESWNVTMVGLDLTHQALATPAVQKRVKEVG
TKPAAFMLQILDFYTKVYEKERNTYATVHDPCAVAYVIDPTVMTTEQVPVDIELNGALTTGMTVADFRYPRPKHCHTQVAVKLDFDKFWCL
VIDALKRIGDPQSAVGNIESQWARAGHGLVSLSEQQLVSCDDKDNGCNGGLMLQAFEWLLRHMYGIVFTEKSYPYTSGNGDVAECLNSSKL
VPGAQIDGYVMIPSNETVMAAWLAENGPIAIAVDASSFMSYQSGVLTSCAGDALNHGVLLVGYNKTGGVPYWVIKNSWGEDWGEKGYVRVV
MGLNACLLSEYPVSAHVPRSLTPGPGTESEERAPKRVTVEQMMCTDMYCREGCKKSLLTANVCYKNGGGGSSMTKCGPQKVLMCSYSNPHC
FGPGLCLETPDGKCAPYFLGSIMNTCQYTMASSRSAPRKASHAHKSHRKPKRSWNVYVGRSLKAINAQMSMSHRTMSIVNSYVNDVMERIC
MEAASIVRANKKRTLGAREVQTAVRIVLPAELAKHAMAEGTKAVSSASA
8MCH: mtHSP70₅₀₉₋₆₆₀ + MDH₁₋₃₂₂ + CPB₁₅₄₋₄₄₃ + H2B₁₋₁₁₁
8E (1 . . . 459) + MDH (460 . . . 1425) + CPB (1426 . . . 2295) + H2Bn (2296 . . . 2629)
MW = 93806 Daltons

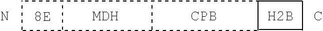

SEQ ID NO: 44 Polynucleotide encoding the 8MCH Fusion Polypeptide
atgAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGGTAAACGTGTGCGTTGTTGGGGCTGCCGGCGGCATCGGGCAGTCGCTGTCGCTTCTGCTGGTGCGCCAGCTGCCGTACGGGAGC
ACGTTGTCGTTGTTCGACGTTGTGGGCGCTGCCGGCGTTGCAGCGGACCTGTCGCACGTGGACAACGCCGGTGTGCAGGTGAAGTTCGCGG
CGGGCAAGATAGGCCAGAAGCGCGACCCTGCGCTAGCGGAGCTTGCGAAGGGCGTGGATGTGTTTGTGATGGTGGCTGGCGTGCCACGCAA
GCCGGGCATGACGCGCGACGACCTTTTCAAAATCAACGCCGGAATCATCCTGGACCTTGTGCTGACGTGCGCATCGTCGAGCCCAAAGGCG
GTGTTCTGCATTGTGACGAACCCTGTGAACAGCACGGTCGTGATCGCGGCAGAGGCGCTGAAGAGCCTCGGCGTATACGACAGAAACCGGC
TGCTTGGCGTGTCGCTGCTAGACGGGCTGCGCGCGACGTGCTTCATCAACGAGGCGCGCAAGCCTTTGGTCGTGACGCAGGTGCCAGTTGT
TGGCGGGCACAGCGACGCAACGATTGTTCCGTTGTTCCACCAGCTGCTGGGGCCGTTGCCGGAGCAGGCGACGCTGGACAAGATCGTGAAG
CGCGTGCAGGTTGCAGGCACAGAGGTGGTGAAGGCGAAGGCCGGGCGCGGGTCTGCGACGCTGTCGATGGCGGAGGCTGGCGCGCGGTTCA
CGCTGAAGGTTGTGGAGGGCCTGACCGGCACGGGTAAACCGCTGGTGTACGCATACGTGGACACAGACGGGCAGCACGAGACGCCGTTCCT
CGCGATCCCCGTGGTGCTTGGCGTGAATGGAATCGAGAAGCGCCTGCCAATCGGTCCGCTGCACTCGACAGAGGAAACGCTGCTGAAGGCG
GCACTGCCGGTGATCAAGAAGAATATCGTGAAGGGCAGCGAGTTCGCGCGCTCACACCTGTCGGCGGTCGGCAACATCGAGTCGCAGTGGG
CCCGTGCCGGCCACGGCTTGGTGAGCCTGTCGGAGCAGCAGCTGGTGAGCTGCGATGACAAAGACAATGGCTGCAACGGCGGGCTGATGCT
GCAGGCGTTCGAGTGGCTGCTGCGACACATGTACGGGATCGTGTTCACGGAGAAGAGCTACCCCTACACGTCCGGCAACGGTGATGTGGCC
GAGTGCTTGAACAGCAGTAAACTCGTTCCCGGCGCGCAAATCGACGGCTACGTGATGATCCCGAGCAACGAAACGGTTATGGCTGCGTGGC
TTGCGGAGAATGGCCCCATCGCGATTGCGGTCGACGCCAGCTCCTTCATGTCTTACCAGAGCGGCGTGCTGACCAGCTGCGCTGGCGATGC
ACTGAACCACGGCGTGCTGCTCGTCGGGTACAACAAGACCGGTGGGGTTCCGTACTGGGTGATCAAGAACTCGTGGGGTGAGGACTGGGGC
GAGAAGGGCTACGTGCGCGTGGTCATGGGGCTGAACGCGTGCCTGCTCAGTGAATACCCCGTGTCCGCGCATGTGCCGCGGAGTCTCACCC
CTGGCCCGGGCACGGAGAGCGAGGAGCGCGCCCCTAAACGGGTGACGGTGGAGCAGATGATGTGCACCGATATGTACTGCAGGGAGGGGTG
CAAGAAGAGTCTTCTCACCGCGAACGTGTGCTACAAGAACGGGGGAGGCGGCTCCTCTATGACGAAGTGCGGTCCGCAGAAGGTGCTGATG
TGCTCGTACTCGAACCCTCATTGCTTTGGTCCTGGGCTGTGCCTCGAGACTCCTGATGGCAAGTGCGCGCCGTACTTCTTGGGCTCGATCA
TGAACACCTGCCAGTACACGATGGCCTCTTCTCGCTCTGCTCCCCGCAAGGCTTCCCACGCGCACAAGTCGCACCGCAAGCCGAAGCGCTC
GTGGAACGTGTACGTGGGCCGCTCGCTGAAGGCGATCAACGCCCAGATGTCGATGTCGCACCGCACGATGAGCATCGTGAACTCGTACGTG
AACGACGTGATGGAGCGCATCTGCATGGAGGCCGCGTCGATCGTTCGCGCGAACAAGAAGCGCACGTTGGGTGCGCGCGAGGTGCAGACGG
CGGTGCGCATTGTGCTGCCGGCGGAGCTCGCGAAGCACGCCATGGCTGAGGGCACGAAGGCCGTGTCGAGCGCGTCGGCTT
SEQ ID NO: 45 Amino Acid Sequence of the 8MCH Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMVNVCVVGAAGGIGQSLSLLLVRQLPYGS
TLSLFDVVGAAGVAADLSHVDNAGVQVKFAAGKIGQKRDPALAELAKGVDVFVMVAGVPRKPGMTRDDLFKINAGIILDLVLTCASSSPKA
VFCIVTNPVNSTVVIAAEALKSLGVYDRNRLLGVSLLDGLRATCFINEARKPLVVTQVPVVGGHSDATIVPLFHQLLGPLPEQATLDKIVK
RVQVAGTEVVKAKAGRGSATLSMAEAGARFTLKVVEGLTGTGKPLVYAYVDTDGQHETPFLAIPVVLGVNGIEKRLPIGPLHSTEETLLKA
ALPVIKKNIVKGSEFARSHLSAVGNIESQWARAGHGLVSLSEQQLVSCDDKDNGCNGGLMLQAFEWLLRHMYGIVFTEKSYPYTSGNGDVA
ECLNSSKLVPGAQIDGYVMIPSNETVMAAWLAENGPIAIAVDASSFMSYQSGVLTSCAGDALNHGVLLVGYNKTGGVPYWVIKNSWGEDWG
EKGYVRVVMGLNACLLSEYPVSAHVPRSLTPGPGTESEERAPKRVTVEQMMCTDMYCREGCKKSLLTANVCYKNGGGGSSMTKCGPQKVLM
CSYSNPHCFGPGLCLETPDGKCAPYFLGSIMNTCQYTMASSRSAPRKASHAHKSHRKPKRSWNVYVGRSLKAINAQMSMSHRTMSIVNSYV
NDVMERICMEAASIVRANKKRTLGAREVQTAVRIVLPAELAKHAMAEGTKAVSSASA
8MTH: mtHSP70₅₀₉₋₆₆₀ + MDH₁₋₃₂₂ + aT ₁₋₄₉₀ + H2B₁₋₁₁₁
8E (1 . . . 459) + MDH (460 . . . 1425) + aT (1426 . . . 2894) + H2Bn (2295 . . . 3228)
MW = 116,856 Daltons

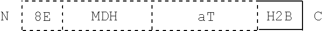

SEQ ID NO: 46 Polynucleotide encoding the 8MTH Fusion Polypeptide
atgAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGGTAAACGTGTGCGTTGTTGGGGCTGCCGGCGGCATCGGGCAGTCGCTGTCGCTTCTGCTGGTGCGCCAGCTGCCGTACGGGAGC
ACGTTGTCGTTGTTCGACGTTGTGGGCGCTGCCGGCGTTGCAGCGGACCTGTCGCACGTGGACAACGCCGGTGTGCAGGTGAAGTTCGCGG
CGGGCAAGATAGGCCAGAAGCGCGACCCTGCGCTAGCGGAGCTTGCGAAGGGCGTGGATGTGTTTGTGATGGTGGCTGGCGTGCCACGCAA
GCCGGGCATGACGCGCGACGACCTTTTCAAAATCAACGCCGGAATCATCCTGGACCTTGTGCTGACGTGCGCATCGTCGAGCCCAAAGGCG
GTGTTCTGCATTGTGACGAACCCTGTGAACAGCACGGTCGTGATCGCGGCAGAGGCGCTGAAGAGCCTCGGCGTATACGACAGAAACCGGC
TGCTTGGCGTGTCGCTGCTAGACGGGCTGCGCGCGACGTGCTTCATCAACGAGGCGCGCAAGCCTTTGGTCGTGACGCAGGTGCCAGTTGT
TGGCGGGCACAGCGACGCAACGATTGTTCCGTTGTTCCACCAGCTGCTGGGGCCGTTGCCGGAGCAGGCGACGCTGGACAAGATCGTGAAG
CGCGTGCAGGTTGCAGGCACAGAGGTGGTGAAGGCGAAGGCCGGGCGCGGGTCTGCGACGCTGTCGATGGCGGAGGCTGGCGCGCGGTTCA
CGCTGAAGGTTGTGGAGGGCCTGACCGGCACGGGTAAACCGCTGGTGTACGCATACGTGGACACAGACGGGCAGCACGAGACGCCGTTCCT
CGCGATCCCCGTGGTGCTTGGCGTGAATGGAATCGAGAAGCGCCTGCCAATCGGTCCGCTGCACTCGACAGAGGAAACGCTGCTGAAGGCG
GCACTGCCGGTGATCAAGAAGAATATCGTGAAGGGCAGCGAGTTCGCGCGCTCACACCTGATGCGCGATGCACACACGCGCACGCCCACCG
AAAAAAAAACGCGCAGCTCTTCGCTCTCGTTCTTCGAACAAACACCTTTAAACCGCCTTCTAACCCCTCTTTCTTCTTTTTCAGCCATGCG
TGAGGCTATCTGCATCCACATCGGCCAGGCCGGCTGCCAGGTCGGTAACGCGTGCTGGGAGCTGTTCTGCCTTGAGCACGGCATCCAGCCT
GATGGCTCCATGCCCTCTGACAAGTGCATCGGTGTTGAGGATGACGCGTTCAACACGTTCTTCTCGGAGACTGGTGCTGGCAAGCACGTTC
CTCGCTGCATCTTCCTGGACCTCGAGCCTACGGTCGTGGATGAGGTGCGCACCGGCACGTACCGCCAGCTGTTCAACCCCGAGCAGCTGGT
GTCCGGCAAGGAGGATGCGGCGAACAACTACGCTCGTGGCCACTACACCATCGGCAAGGAGATCGTCGACCTTGCGCTGGACCGCATTCGC
AAGCTGGCGGACAACTGCACGGGTCTCCAGGGCTTTATGGTGTTCCACGCTGTGGGTGGCGGCACCGGCTCTGGCCTCGGTGCGCTGCTGC
TGGAGCGCCTGTCTGTGGACTACGGCAAGAAGTCCAAGCTTGGCTACACCGTGTACCCGAGCCCGCAGGTGTCGACTGCCGTCGTGGAGCC
GTACAACTGCGTGCTGTCGACGCACTCGCTGCTCGAGCACACCGATGTTGCGACGATGCTCGACAATGAGGCCATCTACGACCTCACTCGT
CGTTCTCTCGACATTGAGCGCCCGTCGTACACGAACGTGAACCGCCTGATCGGCCAGGTGGTGTCGTCTCTGACGGCGTCGCTGCGCTTCG
ATGGTGCGCTGAACGTGGACCTGACGGAGTTCCAGACGAACCTTGTGCCGTACCCGCGCATCCACTTCGTGCTGACGAGCTATGCTCCGGT
GGTGTCTGCCGAGAAGGCGTACCACGAGCAGCTGTCCGTCGCGGACATCACGAACTCGGTGTTTGAGCCTGCTGGCATGCTGACGAAGTGC
GATCCTCGCCACGGCAAGTACATGTCGTGCTGCCTCATGTACCGCGGTGATGTCGTGCCGAAGGATGTCAACGCCGCGATTGCGACGATCA
CAAGACGAAGCGGACAATTCAGTTCGTGGACTGGTGTCCGACCGGCTTCAAGTGCGGCATCAACTACCAGCCGCCGACCGTTGTGCCCGGC
GGTGACCTCGCGAAGGTGCAGCGCGCCGTGTGCATGATTGCCAACTCGACCGCGATCGCTGAGGTGTTTGCCCGCATCGACCACAAGTTCG
ACCTGATGTACAGCAAGCGCGCGTTCGTGCACTGGTACGTGGGTGAGGGCATGGAGGAGGGCGAGTTCTCCGAGGCGCGCGAGGATCTCGC
TGCGCTGGAGAAGGACTACGAGGAGGTTGGCGCTGAGTCCGCCGACGACATGGGTGAGGAGGACGTCGAGGAGTACATGGCCTCTTCTCGC
TCTGCTCCCCGCAAGGCTTCCCACGCGCACAAGTCGCACCGCAAGCCGAAGCGCTCGTGGAACGTGTACGTGGGCCGCTCGCTGAAGGCGA
TCAACGCCCAGATGTCGATGTCGCACCGCACGATGAGCATCGTGAACTCGTACGTGAACGACGTGATGGAGCGCATCTGCATGGAGGCCGC
GTCGATCGTTCGCGCGAACAAGAAGCGCACGTTGGGTGCGCGCGAGGTGCAGACGGCGGTGCGCATTGTGCTGCCGGCGGAGCTCGCGAAG
CACGCCATGGCTGAGGGCACGAAGGCCGTGTCGAGCGCGTCGGCT
SEQ ID NO: 47 Amino Acid Sequence of the 8MTH Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMVNVCVVGAAGGIGQSLSLLLVRQLPYGS
TLSLFDVVGAAGVAADLSHVDNAGVQVKFAAGKIGQKRDPALAELAKGVDVFVMVAGVPRKPGMTRDDLFKINAGIILDLVLTCASSSPKA
VFCIVTNPVNSTVVIAAEALKSLGVYDRNRLLGVSLLDGLRATCFINEARKPLVVTQVPVVGGHSDATIVPLFHQLLGPLPEQATLDKIVK
RVQVAGTEVVKAKAGRGSATLSMAEAGARFTLKVVEGLTGTGKPLVYAYVDTDGQHETPFLAIPVVLGVNGIEKRLPIGPLHSTEETLLKA
ALPVIKKNIVKGSEFARSHLMRDAHTRTPTEKKTRSSSLSFFEQTPLNRLLTPLSSFSAMREAICIHIGQAGCQVGNACWELFCLEHGIQP
DGSMPSDKCIGVEDDAFNTFFSETGAGKHVPRCIFLDLEPTVVDEVRTGTYRQLFNPEQLVSGKEDAANNYARGHYTIGKEIVDLALDRIR
KLADNCTGLQGFMVFHAVGGGTGSGLGALLLERLSVDYGKKSKLGYTVYPSPQVSTAVVEPYNCVLSTHSLLEHTDVATMLDNEAIYDLTR
RSLDIERPSYTNVNRLIGQVVSSLTASLRFDGALNVDLTEFQTNLVPYPRIHFVLTSYAPVVSAEKAYHEQLSVADITNSVFEPAGMLTKC
DPRHGKYMSCCLMYRGDVVPKDVNAAIATIKTKRTIQFVDWCPTGFKCGINYQPPTVVPGGDLAKVQRAVCMIANSTAIAEVFARIDHKFD
LMYSKRAFVHWYVGEGMEEGEFSEAREDLAALEKDYEEVGAESADDMGEEDVEEYMASSRSAPRKASHAHKSHRKPKRSWNVYVGRSLKAI
NAQMSMSHRTMSIVNSYVNDVMERICMEAASIVRANKKRTLGAREVQTAVRIVLPAELAKHAMAEGTKAVSSASA
8TCH: mtHSP70₅₀₉₋₆₆₀ + aT ₁₋₄₉₀ + CPB₁₅₄₋₄₄₃ + H2B₁₋₁₁₁
8E (1 . . . 459) + aT (460 . . . 1929) + CPB (1930 . . . 2799) + H2Bn (2800 . . . 3132)
MW = 114,413 Daltons

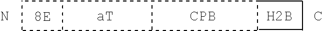

SEQ ID NO: 48 Polynucleotide encoding the 8TCH Fusion Polypeptide
atgAAGGACAAGGCGACGGGCAAGACGCAGAACATCACGATCACGGCGAACGGCGGGCTGTCGAAGGAGCAGATCGAGCAGATGATCCGCG
ACTCGGAGCAGCACGCGGAGGCCGACCGCGTGAAGCGCGAGCTTGTGGAGGTGCGCAACAACGCGGAGACGCAGCTGACAACGGCGGAGAG
GCAGCTCGGCGAGTGGAAGTACGTGAGCGATGCGGAGAAGGAGAACGTGAAGACGCTGGTGGCGGAGCTGCGCAAGGCGATGGAGAACCCG
AACGTCGCGAAGGATGACCTTGCGGCTGCGACGGACAAGCTGCAGAAGGCTGTGATGGAGTGCGGCCGCACAGAGTACCAGCAGGCTGCCG
CGGCCAACTCCGGCAGCACCAGCAACTCCGGTGAGCAGCAGCAGCAGCAGGGCCAAGGTGAGCAGCAGCAGCAGCAGAACAGCGAAGAGAA
GAAGATGCGCGATGCACACACGCGCACGCCCACCGAAAAAAAAACGCGCAGCTCTTCGCTCTCGTTCTTCGAACAAACACCTTTAAACCGC
CTTCTAACCCCTCTTTCTTCTTTTTCAGCCATGCGTGAGGCTATCTGCATCCACATCGGCCAGGCCGGCTGCCAGGTCGGTAACGCGTGCT
GGGAGCTGTTCTGCCTTGAGCACGGCATCCAGCCTGATGGCTCCATGCCCTCTGACAAGTGCATCGGTGTTGAGGATGACGCGTTCAACAC
GTTCTTCTCGGAGACTGGTGCTGGCAAGCACGTTCCTCGCTGCATCTTCCTGGACCTCGAGCCTACGGTCGTGGATGAGGTGCGCACCGGC
ACGTACCGCCAGCTGTTCAACCCCGAGCAGCTGGTGTCCGGCAAGGAGGATGCGGCGAACAACTACGCTCGTGGCCACTACACCATCGGCA
AGGAGATCGTCGACCTTGCGCTGGACCGCATTCGCAAGCTGGCGGACAACTGCACGGGTCTCCAGGGCTTTATGGTGTTCCACGCTGTGGG
TGGCGGCACCGGCTCTGGCCTCGGTGCGCTGCTGCTGGAGCGCCTGTCTGTGGACTACGGCAAGAAGTCCAAGCTTGGCTACACCGTGTAC
CCGAGCCCGCAGGTGTCGACTGCCGTCGTGGAGCCGTACAACTGCGTGCTGTCGACGCACTCGCTGCTCGAGCACACCGATGTTGCGACGA
TGCTCGACAATGAGGCCATCTACGACCTCACTCGTCGTTCTCTCGACATTGAGCGCCCGTCGTACACGAACGTGAACCGCCTGATCGGCCA
GGTGGTGTCGTCTCTGACGGCGTCGCTGCGCTTCGATGGTGCGCTGAACGTGGACCTGACGGAGTTCCAGACGAACCTTGTGCCGTACCCG
CGCATCCACTTCGTGCTGACGAGCTATGCTCCGGTGGTGTCTGCCGAGAAGGCGTACCACGAGCAGCTGTCCGTCGCGGACATCACGAACT
CGGTGTTTGAGCCTGCTGGCATGCTGACGAAGTGCGATCCTCGCCACGGCAAGTACATGTCGTGCTGCCTCATGTACCGCGGTGATGTCGT
GCCGAAGGATGTCAACGCCGCGATTGCGACGATCAAGACGAAGCGGACAATTCAGTTCGTGGACTGGTGTCCGACCGGCTTCAAGTGCGGC
ATCAACTACCAGCCGCCGACCGTTGTGCCCGGCGGTGACCTCGCGAAGGTGCAGCGCGCCGTGTGCATGATTGCCAACTCGACCGCGATCG
CTGAGGTGTTTGCCCGCATCGACCACAAGTTCGACCTGATGTACAGCAAGCGCGCGTTCGTGCACTGGTACGTGGGTGAGGGCATGGAGGA
GGGCGAGTTCTCCGAGGCGCGCGAGGATCTCGCTGCGCTGGAGAAGGACTACGAGGAGGTTGGCGCTGAGTCCGCCGACGACATGGGTGAG
GAGGACGTCGAGGAGTACTCGGCGGTCGGCAACATCGAGTCGCAGTGGGCCCGTGCCGGCCACGGCTTGGTGAGCCTGTCGGAGCAGCAGC
TGGTGAGCTGCGATGACAAAGACAATGGCTGCAACGGCGGGCTGATGCTGCAGGCGTTCGAGTGGCTGCTGCGACACATGTACGGGTTCAC
GGAGAAGAGCTACCCCTACACGTCCGGCAACGGTGATGTGGCCGAGTGCTTGAACAGCAGTAAACTCGTTCCCGGCGCGCAAATCGACGGC
TACGTGATGATCCCGAGCAACGAAACGGTTATGGCTGCGTGGCTTGCGGAGAATGGCCCCATCGCGATTGCGGTCGACGCCAGCTCCTTCA
TGTCTTACCAGAGCGGCGTGCTGACCAGCTGCGCTGGCGATGCACTGAACCACGGCGTGCTGCTCGTCGGGTACAACAAGACCGGTGGGGT
TCCGTACTGGGTGATCAAGAACTCGTGGGGTGAGGACTGGGGCGAGAAGGGCTACGTGCGCGTGGTCATGGGGCTGAACGCGTGCCTGCTC
AGTGAATACCCCGTGTCCGCGCATGTGCCGCGGAGTCTCACCCCTGGCCCGGGCACGGAGAGCGAGGAGCGCGCCCCTAAACGGGTGACGG
TGGAGCAGATGATGTGCACCGATATGTACTGCAGGGAGGGGTGCAAGAAGAGTCTTCTCACCGCGAACGTGTGCTACAAGAACGGGGGAGG
CGGCTCCTCTATGACGAAGTGCGGTCCGCAGAAGGTGCTGATGTGCTCGTACTCGAACCCTCATTGCTTTGGTCCTGGGCTGTGCCTCGAG
ACTCCTGATGGCAAGTGCGCGCCGTACTTCTTGGGCTCGATCATGAACACCTGCCAGTACACGATGGCCTCTTCTCGCTCTGCTCCCCGCA
AGGCTTCCCACGCGCACAAGTCGCACCGCAAGCCGAAGCGCTCGTGGAACGTGTACGTGGGCCGCTCGCTGAAGGCGATCAACGCCCAGAT
GTCGATGTCGCACCGCACGATGAGCATCGTGAACTCGTACGTGAACGACGTGATGGAGCGCATCTGCATGGAGGCCGCGTCGATCGTTCGC
GCGAACAAGAAGCGCACGTTGGGTGCGCGCGAGGTGCAGACGGCGGTGCGCATTGTGCTGCCGGCGGAGCTCGCGAAGCACGCCATGGCTG
AGGGCACGAAGGCCGTGTCGAGCGCGTCGGCT
SEQ ID NO: 49 Amino Acid Sequence of the 8TCH Fusion Polypeptide
MKDKATGKTQNITITANGGLSKEQIEQMIRDSEQHAEADRVKRELVEVRNNAETQLTTAERQLGEWKYVSDAEKENVKTLVAELRKAMENP
NVAKDDLAAATDKLQKAVMECGRTEYQQAAAANSGSTSNSGEQQQQQGQGEQQQQQNSEEKKMRDAHTRTPTEKKTRSSSLSFFEQTPLNR
LLTPLSSFSAMREAICIHIGQAGCQVGNACWELFCLEHGIQPDGSMPSDKCIGVEDDAFNTFFSETGAGKHVPRCIFLDLEPTVVDEVRTG
TYRQLFNPEQLVSGKEDAANNYARGHYTIGKEIVDLALDRIRKLADNCTGLQGFMVFHAVGGGTGSGLGALLLERLSVDYGKKSKLGYTVY
PSPQVSTAVVEPYNCVLSTHSLLEHTDVATMLDNEAIYDLTRRSLDIERPSYTNVNRLIGQVVSSLTASLRFDGALNVDLTEFQTNLVPYP
RIHFVLTSYAPVVSAEKAYHEQLSVADITNSVFEPAGMLTKCDPRHGKYMSCCLMYRGDVVPKDVNAAIATIKTKRTIQFVDWCPTGFKCG
INYQPPTVVPGGDLAKVQRAVCMIANSTAIAEVFARIDHKFDLMYSKRAFVHWYVGEGMEEGEFSEAREDLAALEKDYEEVGAESADDMGE
EDVEEYSAVGNIESQWARAGHGLVSLSEQQLVSCDDKDNGCNGGLMLQAFEWLLRHMYGIVFTEKSYPYTSGNGDVAECLNSSKLVPGAQI
DGYVMIPSNETVMAAWLAENGPIAIAVDASSFMSYQSGVLTSCAGDALNHGVLLVGYNKTGGVPYWVIKNSWGEDWGEKGYVRVVMGLNAC
LLSEYPVSAHVPRSLTPGPGTESEERAPKRVTVEQMMCTDMYCREGCKKSLLTANVCYKNGGGGSSMTKCGPQKVLMCSYSNPHCFGPGLC
LETPDGKCAPYFLGSIMNTCQYTMASSRSAPRKASHAHKSHRKPKRSWNVYVGRSLKAINAQMSMSHRTMSIVNSYVNDVMERICMEAASI
VRANKKRTLGAREVQTAVRIVLPAELAKHAMAEGTKAVSSASA

1: An isolated polypeptide comprising an immunogenic portion of a Leishmania putative mitochondrial heat shock protein 70 (mtHSP70), wherein the immunogenic portion comprises a carboxy terminal region sequence of the mtHSP70.

2.-3. (canceled)

4: The polypeptide of claim 1 wherein the polypeptide is a fusion polypeptide.

5: The polypeptide of claim 4, wherein the polypeptide further comprises a Leishmania carboxypeptidase (CxP) polypeptide.

6.-7. (canceled)

8: The polypeptide of claim 4, wherein the fusion polypeptide further comprises one or more polypeptides selected from the group consisting of a Leishmania cysteine proteinase B (CpB) polypeptide, a Leishmania histone of H2BN (H2BN) polypeptide, a Leishmania A2 polypeptide, a Leishmania p21 antigen (p21) polypeptide, a Leishmania non-specific nucleoside hydrolase (NH) polypeptide, a Leishmania aT polypeptide, and a Leishmania MDH polypeptide.

9: The polypeptide of claim 8, wherein the fusion polypeptide comprises:
(a) a Leishmania mtHSP70 polypeptide, a Leishmania CxP polypeptide, and a Leishmania p21 polypeptide;
(b) a Leishmania mtHSP70 polypeptide, a Leishmania CxP polypeptide, a Leishmania H2BN, and a Leishmania p21 polypeptide;
(c) a Leishmania mtHSP70 polypeptide, a Leishmania CxP polypeptide, a Leishmania A2 polypeptide, and a Leishmania p21 polypeptide;
(d) a Leishmania mtHSP70 polypeptide, a Leishmania A2 polypeptide, a Leishmania p21 polypeptide, and a Leishmania NH polypeptide;
(e) a Leishmania mtHSP70 polypeptide, a Leishmania CxP polypeptide, a Leishmania H2BN polypeptide, and a Leishmania A2 polypeptide;
(f) a Leishmania mtHSP70 polypeptide, a Leishmania H2BN polypeptide, a Leishmania A2 polypeptide, and a Leishmania NH polypeptide;
(g) a Leishmania mtHSP70 polypeptide, a Leishmania CpB polypeptide, a Leishmania H2BN, and a Leishmania A2 polypeptide;
(h) a Leishmania mtHSP70 polypeptide, a Leishmania CpB polypeptide, a Leishmania A2 polypeptide, and a Leishmania NH polypeptide;
(i) a Leishmania mtHSP70 polypeptide, a Leishmania NH polypeptide, and a Leishmania CpB polypeptide;
(j) a Leishmania mtHSP70 polypeptide, a Leishmania NH polypeptide, a Leishmania CpB polypeptide, and a Leishmania H2BN;
(k) a Leishmania mtHSP70 polypeptide, a Leishmania MDH polypeptide, a Leishmania CpB polypeptide, and a Leishmania H2BN polypeptide;
(l) a Leishmania mtHSP70 polypeptide, a Leishmania MDH polypeptide, a Leishmania aT polypeptide, and a Leishmania H2BN polypeptide; or
(m) a Leishmania mtHSP70 polypeptide, a Leishmania aT polypeptide, a Leishmania CpB polypeptide, and a Leishmania H2BN polypeptide.

10.-24. (canceled)

25: The polypeptide of claim 1, wherein the polypeptide comprises the amino acid sequence of SEQ ID NO:2, 4, 6, 8, 14, 16, 18, 20, 41, 43, 45, 47, or 49, or an amino acid sequence having at least 90% identity to SEQ ID NO: 2, 4, 6, 8, 14, 16, 18, 20, 41, 43, 45, 47, or 49.

26: A fusion polypeptide comprising a Leishmania CxP polypeptide and a second Leishmania polypeptide.

27.-28. (canceled)

29: The fusion polypeptide of claim 26, wherein the second polypeptide comprises a sequence of a Leishmania H2BN polypeptide and a Leishmania NH polypeptide.

30.-34. (canceled)

35: The fusion polypeptide of claim 26, wherein the second polypeptide comprises a sequence of a Leishmania TSA polypeptide and a Leishmania Leif polypeptide.

36.-40. (canceled)

41: An isolated polynucleotide encoding the polypeptide of claim 1.

42. (canceled)

43: A composition comprising the polypeptide of claim 1 and an immunostimulant.

44: A composition comprising a Leishmania CxP polypeptide.

45. (canceled)

46: The composition of claim 44, wherein the CxP polypeptide comprises the amino acid sequence of SEQ ID NO:25, 26, 27, 28, or 29, or a sequence having at least 90% identity to SEQ ID NO: 25, 26, 27, 28, or 29.

47: The composition of claim 43, wherein the immunostimulant is selected from the group consisting of a CpG-containing oligonucleotide, synthetic lipid A, MPLTM, 3D-MPLTM, saponins, saponin mimetics, AGPs, Toll-like receptor agonists, or a combination thereof.

48.-50. (canceled)

51: A method for stimulating an immune response against Leishmania in a mammal comprising administering to a mammal in need thereof a composition according to claim 43.

52: A method for detecting Leishmania infection in a biological sample, comprising: (a) contacting a biological sample with the polypeptide of claim 1; and (b) detecting in the biological sample the presence of antibodies that bind to the polypeptide, thereby detecting Leishmania infection in a biological sample.

53: A method for detecting Leishmania infection in a biological sample, comprising: (a) contacting a biological sample with a Leishmania CxP polypeptide; and (b) detecting in the biological sample the presence of antibodies that bind to the polypeptide, thereby detecting Leishmania infection in a biological sample.

54.-55. (canceled)

56: A diagnostic reagent comprising a polypeptide of claim 1 and/or a Leishmania CxP polypeptide, wherein the polypeptide is immobilized on a solid support.

57: A diagnostic kit for detecting Leishmania infection in a biological sample comprising (i) a polypeptide of claim 1 and/or a Leishmania CxP polypeptide; and (ii) a detection reagent.

58. (canceled)

59: A point of care diagnostic kit for detecting Leishmania infection in a biological sample comprising a polypeptide of claim 1 and/or a Leishmania CxP polypeptide, wherein the polypeptide is immobilized on a solid support in a lateral flow test strip format.


SEQUENCE LISTING (50 sequences)

SEQ ID NO: 1 - DNA, 2541 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgagcattatcaaggaggacgacgccgtgggctgctacatgacggtgaccctcgtggacgacaccaaggtggagggtaccatcttcacctacaatcccaaggaaggcatcatagtacttctgtccctccgcgacgatcagacgaacatgaagctgatccgcactccatacatcaaagagttcagtatttcacacgctgaggagggaacgcacctgcctccggcactggactccttcaacgagcttccgtccatgcatgccggccgcgacaagtccatcttcaagcacgccagcacgcagctcaagaacgccgaggcgaaccgcgaaaagcacttcaactctgtcacgaccgacacaccgattgccacactcgatgcgtacctcaagctcctgcggctataccccttcattgagtggaacagcgacgagggtgtcatccaggtctcggataccgtcattgtcgtaggggaccccgactggcggacgcccaaggcgatgctggtagacggcgcccctgagaaggacagaccgctcgtagaccgcctgcaggttgcgctcggaaacggcaagaagatgcaggcctacacacaactggagaagctctgccagaaggtgtacagattggcgcaccttctgtctctcggcgcttgggattccaagactatgatgccctcaaagggcgcagctgcccgcggtgccgccctcggcgagctctacggactcatcgctgagatgatcaccagcccgagcacgaaggcgctgctggacgaagcagagacggccaaggccgagctcactactgtccagcaggcgaacttgcgcgagctccgccgcatgtacacctctcaagcagcgctaccgaccgagttcagtgtgctcaagaccaagctttcgtcaactactccgcttatctgggttaagtgccgcagcaacaacgactttgcgactttcctgccggcgctgaaggagatgattgcgcttgcgcgcagggaggcgcagtatcgctctactgcgacgggcaagcctctgtacgaggccctgttcaaccagtacgagagcggcatgacgctggagacgctggaaaaaatcttgctcgatgtgaagtcgtggctgccggagctgctgcagaagatcctggctgcacagagggacgcggggctggaggtggttgcgcctgaggcgccctttcccaaggacaagcaggaggctcttagccgccacctcatggaggtgtggggcttcgacttcgagtcaggtcggctggacgtctctgagcacccgtttatgggcatggtaaaggaagactcgcgcatcactaccgcctacgacctgcaggacttcaccaaggggctcttcgcgacgatccacgagacgggccactccaagtacgagacgaactgcggcccggtggagatgcgcggccagccggtgtgcgaggcacgctcgatgacgatccacgagagccagtcgcgctttgccgaggttgtgattggccactccagcgccttcttggagttcctcgttccactgctgaaggaatacctcggtgatcagcccgcattctctcgggagaacgtgcggctgatgaaccagacggtgaagcctggcttcatccggatccgggcggatgaggtgtgctacccgctgcacatcttgctgcgctacgagatagagcgtgcactcatcgagggcacgatggaggcagaagacatccctcgcgtgtggaacgagaagatgaaggcatacctgggcctggagacggagggccgcgacgagattggctgcctgcaggacattcactggtcgatgggcgcctttggctacttcccgacgtactcgcttggctccatgttcgcggcgcagctgatggcgacgatcaagaatgagctcggtgaggatacagtggacaagtgcatccgcactggccagatggagccgatctttgagaagcagagggagaagatctggagccagggatgcctctacaacacggaagacctgattgtcaaggcgaccggcgaagcgctgaaccccaagtactttcgcgagtacctggaacgccgctacctgcgccaggaggac

SEQ ID NO: 2 - protein, 847 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMSIIKEDDAVGCYMTVTLVDDTKVEGTIFTYNPKEGIIVLLSLRDDQTNMKLIRTPYIKEFSISHAEEGTHLPPALDSFNELPSMHAGRDKSIFKHASTQLKNAEANREKHFNSVTTDTPIATLDAYLKLLRLYPFIEWNSDEGVIQVSDTVIVVGDPDWRTPKAMLVDGAPEKDRPLVDRLQVALGNGKKMQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQED

SEQ ID NO: 3 - DNA, 2679 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgagcattatcaaggaggacgacgccgtgggctgctacatgacggtgaccctcgtggacgacaccaaggtggagggtaccatcttcacctacaatcccaaggaaggcatcatagtacttctgtccctccgcgacgatcagacgaacatgaagctgatccgcactccatacatcaaagagttcagtatttcacacgctgaggagggaacgcacctgcctccggcactggactccttcaacgagcttccgtccatgcatgccggccgcgacaagtccatcttcaagcacgccagcacgcagctcaagaacgccgaggcgaaccgcgaaaagcacttcaactctgtcacgaccgacacaccgattgccacactcgatgcgtacctcaagctcctgcggctataccccttcattgagtggaacagcgacgagggtgtcatccaggtctcggataccgtcattgtcgtaggggaccccgactggcggacgcccaaggcgatgctggtagacggcgcccctgagaaggacagaccgctcgtagaccgcctgcaggttgcgctcggaaacggcaagaagatgcaggcctacacacaactggagaagctctgccagaaggtgtacagattggcgcaccttctgtctctcggcgcttgggattccaagactatgatgccctcaaagggcgcagctgcccgcggtgccgccctcggcgagctctacggactcatcgctgagatgatcaccagcccgagcacgaaggcgctgctggacgaagcagagacggccaaggccgagctcactactgtccagcaggcgaacttgcgcgagctccgccgcatgtacacctctcaagcagcgctaccgaccgagttcagtgtgctcaagaccaagctttcgtcaactactccgcttatctgggttaagtgccgcagcaacaacgactttgcgactttcctgccggcgctgaaggagatgattgcgcttgcgcgcagggaggcgcagtatcgctctactgcgacgggcaagcctctgtacgaggccctgttcaaccagtacgagagcggcatgacgctggagacgctggaaaaaatcttgctcgatgtgaagtcgtggctgccggagctgctgcagaagatcctggctgcacagagggacgcggggctggaggtggttgcgcctgaggcgccctttcccaaggacaagcaggaggctcttagccgccacctcatggaggtgtggggcttcgacttcgagtcaggtcggctggacgtctctgagcacccgtttatgggcatggtaaaggaagactcgcgcatcactaccgcctacgacctgcaggacttcaccaaggggctcttcgcgacgatccacgagacgggccactccaagtacgagacgaactgcggcccggtggagatgcgcggccagccggtgtgcgaggcacgctcgatgacgatccacgagagccagtcgcgctttgccgaggttgtgattggccactccagcgccttcttggagttcctcgttccactgctgaaggaatacctcggtgatcagcccgcattctctcgggagaacgtgcggctgatgaaccagacggtgaagcctggcttcatccggatccgggcggatgaggtgtgctacccgctgcacatcttgctgcgctacgagatagagcgtgcactcatcgagggcacgatggaggcagaagacatccctcgcgtgtggaacgagaagatgaaggcatacctgggcctggagacggagggccgcgacgagattggctgcctgcaggacattcactggtcgatgggcgcctttggctacttcccgacgtactcgcttggctccatgttcgcggcgcagctgatggcgacgatcaagaatgagctcggtgaggatacagtggacaagtgcatccgcactggccagatggagccgatctttgagaagcagagggagaagatctggagccagggatgcctctacaacacggaagacctgattgtcaaggcgaccggcgaagcgctgaaccccaagtactttcgcgagtacctggaacgccgctacctgcgccaggaggacatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacg

SEQ ID NO: 4 - protein, 893 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMSIIKEDDAVGCYMTVTLVDDTKVEGTIFTYNPKEGIIVLLSLRDDQTNMKLIRTPYIKEFSISHAEEGTHLPPALDSFNELPSMHAGRDKSIFKHASTQLKNAEANREKHFNSVTTDTPIATLDAYLKLLRLYPFIEWNSDEGVIQVSDTVIVVGDPDWRTPKAMLVDGAPEKDRPLVDRLQVALGNGKKMQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQEDMASSRSAPRKASHAHKSHRKPKRSWNVYVGRSLKAINAQMSMSHRT

SEQ ID NO: 5 - DNA, 3183 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgagcattatcaaggaggacgacgccgtgggctgctacatgacggtgaccctcgtggacgacaccaaggtggagggtaccatcttcacctacaatcccaaggaaggcatcatagtacttctgtccctccgcgacgatcagacgaacatgaagctgatccgcactccatacatcaaagagttcagtatttcacacgctgaggagggaacgcacctgcctccggcactggactccttcaacgagcttccgtccatgcatgccggccgcgacaagtccatcttcaagcacgccagcacgcagctcaagaacgccgaggcgaaccgcgaaaagcacttcaactctgtcacgaccgacacaccgattgccacactcgatgcgtacctcaagctcctgcggctataccccttcattgagtggaacagcgacgagggtgtcatccaggtctcggataccgtcattgtcgtaggggaccccgactggcggacgcccaaggcgatgctggtagacggcgcccctgagaaggacagaccgctcgtagaccgcctgcaggttgcgctcggaaacggcaagaagatgcaggcctacacacaactggagaagctctgccagaaggtgtacagattggcgcaccttctgtctctcggcgcttgggattccaagactatgatgccctcaaagggcgcagctgcccgcggtgccgccctcggcgagctctacggactcatcgctgagatgatcaccagcccgagcacgaaggcgctgctggacgaagcagagacggccaaggccgagctcactactgtccagcaggcgaacttgcgcgagctccgccgcatgtacacctctcaagcagcgctaccgaccgagttcagtgtgctcaagaccaagctttcgtcaactactccgcttatctgggttaagtgccgcagcaacaacgactttgcgactttcctgccggcgctgaaggagatgattgcgcttgcgcgcagggaggcgcagtatcgctctactgcgacgggcaagcctctgtacgaggccctgttcaaccagtacgagagcggcatgacgctggagacgctggaaaaaatcttgctcgatgtgaagtcgtggctgccggagctgctgcagaagatcctggctgcacagagggacgcggggctggaggtggttgcgcctgaggcgccctttcccaaggacaagcaggaggctcttagccgccacctcatggaggtgtggggcttcgacttcgagtcaggtcggctggacgtctctgagcacccgtttatgggcatggtaaaggaagactcgcgcatcactaccgcctacgacctgcaggacttcaccaaggggctcttcgcgacgatccacgagacgggccactccaagtacgagacgaactgcggcccggtggagatgcgcggccagccggtgtgcgaggcacgctcgatgacgatccacgagagccagtcgcgctttgccgaggttgtgattggccactccagcgccttcttggagttcctcgttccactgctgaaggaatacctcggtgatcagcccgcattctctcgggagaacgtgcggctgatgaaccagacggtgaagcctggcttcatccggatccgggcggatgaggtgtgctacccgctgcacatcttgctgcgctacgagatagagcgtgcactcatcgagggcacgatggaggcagaagacatccctcgcgtgtggaacgagaagatgaaggcatacctgggcctggagacggagggccgcgacgagattggctgcctgcaggacattcactggtcgatgggcgcctttggctacttcccgacgtactcgcttggctccatgttcgcggcgcagctgatggcgacgatcaagaatgagctcggtgaggatacagtggacaagtgcatccgcactggccagatggagccgatctttgagaagcagagggagaagatctggagccagggatgcctctacaacacggaagacctgattgtcaaggcgaccggcgaagcgctgaaccccaagtactttcgcgagtacctggaacgccgctacctgcgccaggaggacagcgcctccgctgagccgcacaaggcggccgttgacgtcggcccgctgagcgttggcccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgctgagcgttggcccgcagagcgttggcccgctgagcgttggcagccagagcgtcggcccgctgagcgttggtccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgcagagcgttggcccgctgagcgttggcagccagagcgtcggcccgctgagcgttggtccgcagagcgtcggcccgctgagcgttggcccgcagagcgtcggcccgctgagcgttggcccgcagagcgtcggcccgctgagcgttggtccgcagagcgttggcccgctgagcgttggcccgcagagcgttgacgttagcccggtgagc

SEQ ID NO: 6 - protein, 1061 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMSIIKEDDAVGCYMTVTLVDDTKVEGTIFTYNPKEGIIVLLSLRDDQTNMKLIRTPYIKEFSISHAEEGTHLPPALDSFNELPSMHAGRDKSIFKHASTQLKNAEANREKHFNSVTTDTPIATLDAYLKLLRLYPFIEWNSDEGVIQVSDTVIVVGDPDWRTPKAMLVDGAPEKDRPLVDRLQVALGNGKKMQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQEDSASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 7 - DNA, 2616 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgagcattatcaaggaggacgacgccgtgggctgctacatgacggtgaccctcgtggacgacaccaaggtggagggtaccatcttcacctacaatcccaaggaaggcatcatagtacttctgtccctccgcgacgatcagacgaacatgaagctgatccgcactccatacatcaaagagttcagtatttcacacgctgaggagggaacgcacctgcctccggcactggactccttcaacgagcttccgtccatgcatgccggccgcgacaagtccatcttcaagcacgccagcacgcagctcaagaacgccgaggcgaaccgcgaaaagcacttcaactctgtcacgaccgacacaccgattgccacactcgatgcgtacctcaagctcctgcggctataccccttcattgagtggaacagcgacgagggtgtcatccaggtctcggataccgtcattgtcgtaggggaccccgactggcggacgcccaaggcgatgctggtagacggcgcccctgagaaggacagaccgctcgtagaccgcctgcaggttgcgctcggaaacggcaagaagatgccgcgcaagattattctcgattgtgatcccgggatcgatgatgccgtggccatctttctcgcccacggcaacccggaggtcgagctgctggccattacgacggtggtgggcaaccagaccctggagaaggtgacccggaacgcgcggctggtagctgacgtagccggcatcgttggtgtgcccgtcgcggctggttgcaccaagcccctcgtgcgcggtgtgcggaatgcctctcagattcatggcgaaaccggcatgggtaacgtctcctacccaccagagttcaagacaaagttggacggccgtcatgcagtgcagctgatcatcgaccttatcatgtcgcacgagccgaagacgatcacgcttgtgcctacgggtggcctgacgaacattgcgatggctgtccgtcttgagccgcgcatcgtggaccgtgtgaaggaggtggttctgatgggtggcggctaccatactggtaatgcgtcccctgtagcggagttcaacgtcttcgtcgacccggaggcggcgcacattgtgttcaacgagagctggaacgtaacgatggtggggctggacctaacgcaccaggcactcgccacgccggcggtccagaagcgagtgaaggaggtgggcacgaagccggctgccttcatgctgcagattttggacttttacacgaaggtgtacgaaaaggagcgcaacacgtacgcgacggtgcacgatccctgcgctgtggcgtacgtgattgaccccaccgtgatgacgacggagcaagtgccagtggacatcgagctcaatggggcactgacgactgggatgacggtcgcggacttccgctacccacggccaaagcactgccacacgcaggtggctgtgaagctggacttcgacaagttttggtgcctcgtgattgacgcactcaagcgcatcggcgatcctcaaagcgcctccgctgagccgcacaaggcggccgttgacgtcggcccgctgagcgttggcccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgctgagcgttggcccgcagagcgttggcccgctgagcgttggcagccagagcgtcggcccgctgagcgttggtccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgcagagcgttggcccgctgagcgttggcagccagagcgtcggcccgctgagcgttggtccgcagagcgtcggcccgctgagcgttggcccgcagagcgtcggcccgctgagcgttggcccgcagagcgtcggcccgctgagcgttggtccgcagagcgttggcccgctgagcgttggcccgcagagcgttgacgttagcccggtgagc

SEQ ID NO: 8 - protein, 872 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMSIIKEDDAVGCYMTVTLVDDTKVEGTIFTYNPKEGIIVLLSLRDDQTNMKLIRTPYIKEFSISHAEEGTHLPPALDSFNELPSMHAGRDKSIFKHASTQLKNAEANREKHFNSVTTDTPIATLDAYLKLLRLYPFIEWNSDEGVIQVSDTVIVVGDPDWRTPKAMLVDGAPEKDRPLVDRLQVALGNGKKMPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQSASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 9 - DNA, 2589 bp, Artificial Sequence
  Synthetic Construct
atgccgcgcaagattattctcgattgtgatcccgggatcgatgatgccgtggccatctttctcgcccacggcaacccggaggtcgagctgctggccattacgacggtggtgggcaaccagaccctggagaaggtgacccggaacgcgcggctggtagctgacgtagccggcatcgttggtgtgcccgtcgcggctggttgcaccaagcccctcgtgcgcggtgtgcggaatgcctctcagattcatggcgaaaccggcatgggtaacgtctcctacccaccagagttcaagacaaagttggacggccgtcatgcagtgcagctgatcatcgaccttatcatgtcgcacgagccgaagacgatcacgcttgtgcctacgggtggcctgacgaacattgcgatggctgtccgtcttgagccgcgcatcgtggaccgtgtgaaggaggtggttctgatgggtggcggctaccatactggtaatgcgtcccctgtagcggagttcaacgtcttcgtcgacccggaggcggcgcacattgtgttcaacgagagctggaacgtaacgatggtggggctggacctaacgcaccaggcactcgccacgccggcggtccagaagcgagtgaaggaggtgggcacgaagccggctgccttcatgctgcagattttggacttttacacgaaggtgtacgaaaaggagcgcaacacgtacgcgacggtgcacgatccctgcgctgtggcgtacgtgattgaccccaccgtgatgacgacggagcaagtgccagtggacatcgagctcaatggggcactgacgactgggatgacggtcgcggacttccgctacccacggccaaagcactgccacacgcaggtggctgtgaagctggacttcgacaagttttggtgcctcgtgattgacgcactcaagcgcatcggcgatcctcaaatgcaggcctacacacaactggagaagctctgccagaaggtgtacagattggcgcaccttctgtctctcggcgcttgggattccaagactatgatgccctcaaagggcgcagctgcccgcggtgccgccctcggcgagctctacggactcatcgctgagatgatcaccagcccgagcacgaaggcgctgctggacgaagcagagacggccaaggccgagctcactactgtccagcaggcgaacttgcgcgagctccgccgcatgtacacctctcaagcagcgctaccgaccgagttcagtgtgctcaagaccaagctttcgtcaactactccgcttatctgggttaagtgccgcagcaacaacgactttgcgactttcctgccggcgctgaaggagatgattgcgcttgcgcgcagggaggcgcagtatcgctctactgcgacgggcaagcctctgtacgaggccctgttcaaccagtacgagagcggcatgacgctggagacgctggaaaaaatcttgctcgatgtgaagtcgtggctgccggagctgctgcagaagatcctggctgcacagagggacgcggggctggaggtggttgcgcctgaggcgccctttcccaaggacaagcaggaggctcttagccgccacctcatggaggtgtggggcttcgacttcgagtcaggtcggctggacgtctctgagcacccgtttatgggcatggtaaaggaagactcgcgcatcactaccgcctacgacctgcaggacttcaccaaggggctcttcgcgacgatccacgagacgggccactccaagtacgagacgaactgcggcccggtggagatgcgcggccagccggtgtgcgaggcacgctcgatgacgatccacgagagccagtcgcgctttgccgaggttgtgattggccactccagcgccttcttggagttcctcgttccactgctgaaggaatacctcggtgatcagcccgcattctctcgggagaacgtgcggctgatgaaccagacggtgaagcctggcttcatccggatccgggcggatgaggtgtgctacccgctgcacatcttgctgcgctacgagatagagcgtgcactcatcgagggcacgatggaggcagaagacatccctcgcgtgtggaacgagaagatgaaggcatacctgggcctggagacggagggccgcgacgagattggctgcctgcaggacattcactggtcgatgggcgcctttggctacttcccgacgtactcgcttggctccatgttcgcggcgcagctgatggcgacgatcaagaatgagctcggtgaggatacagtggacaagtgcatccgcactggccagatggagccgatctttgagaagcagagggagaagatctggagccagggatgcctctacaacacggaagacctgattgtcaaggcgaccggcgaagcgctgaaccccaagtactttcgcgagtacctggaacgccgctacctgcgccaggaggacatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacg

SEQ ID NO: 10 - protein, 863 aa, Artificial Sequence
  Synthetic Construct
MPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQMQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQEDMASSRSAPRKASHAHKSHRKPKRSWNVYVGRSLKAINAQMSMSHRT

SEQ ID NO: 11 - DNA, 2796 bp, Artificial Sequence
  Synthetic Construct
atgtcctgcggtaacgccaagatcaactctcccgcgccgtccttcgaggaggtggcgctcatgcccaacggcagcttcaagaagatcagcctctcctcctacaagggcaagtgggtcgtgctcttcttctacccgctcgacttcaccttcgtgtgcccgacagaggtcatcgcgttctccgacagcgtgagtcgcttcaacgagctcaactgcgaggtcctcgcgtgctcgatagacagcgagtacgcgcacctgcagtggacgctgcaggaccgcaagaagggcggcctcgggaccatggcgatcccaatgctagccgacaagaccaagagcatcgctcgttcctacggcgtgctggaggagagccagggcgtggcctaccgcggtctcttcatcatcgacccccatggcatgctgcgtcagatcaccgtcaatgacatgccggtgggccgcagcgtggaggaggttctacgcctgctggaggcttttcagttcgtggagaagcacggcgaggtgtgccccgcgaactggaagaagggcgcccccacgatgaagccggaaccgaatgcgtctgtcgagggatacttcagcaagcagggatccatgcaggcctacacacaactggagaagctctgccagaaggtgtacagattggcgcaccttctgtctctcggcgcttgggattccaagactatgatgccctcaaagggcgcagctgcccgcggtgccgccctcggcgagctctacggactcatcgctgagatgatcaccagcccgagcacgaaggcgctgctggacgaagcagagacggccaaggccgagctcactactgtccagcaggcgaacttgcgcgagctccgccgcatgtacacctctcaagcagcgctaccgaccgagttcagtgtgctcaagaccaagctttcgtcaactactccgcttatctgggttaagtgccgcagcaacaacgactttgcgactttcctgccggcgctgaaggagatgattgcgcttgcgcgcagggaggcgcagtatcgctctactgcgacgggcaagcctctgtacgaggccctgttcaaccagtacgagagcggcatgacgctggagacgctggaaaaaatcttgctcgatgtgaagtcgtggctgccggagctgctgcagaagatcctggctgcacagagggacgcggggctggaggtggttgcgcctgaggcgccctttcccaaggacaagcaggaggctcttagccgccacctcatggaggtgtggggcttcgacttcgagtcaggtcggctggacgtctctgagcacccgtttatgggcatggtaaaggaagactcgcgcatcactaccgcctacgacctgcaggacttcaccaaggggctcttcgcgacgatccacgagacgggccactccaagtacgagacgaactgcggcccggtggagatgcgcggccagccggtgtgcgaggcacgctcgatgacgatccacgagagccagtcgcgctttgccgaggttgtgattggccactccagcgccttcttggagttcctcgttccactgctgaaggaatacctcggtgatcagcccgcattctctcgggagaacgtgcggctgatgaaccagacggtgaagcctggcttcatccggatccgggcggatgaggtgtgctacccgctgcacatcttgctgcgctacgagatagagcgtgcactcatcgagggcacgatggaggcagaagacatccctcgcgtgtggaacgagaagatgaaggcatacctgggcctggagacggagggccgcgacgagattggctgcctgcaggacattcactggtcgatgggcgcctttggctacttcccgacgtactcgcttggctccatgttcgcggcgcagctgatggcgacgatcaagaatgagctcggtgaggatacagtggacaagtgcatccgcactggccagatggagccgatctttgagaagcagagggagaagatctggagccagggatgcctctacaacacggaagacctgattgtcaaggcgaccggcgaagcgctgaaccccaagtactttcgcgagtacctggaacgccgctacctgcgccaggaggacgaattcatggcgcagaatgataagatcgccccccaggaccaggactccttcctcgatgaccagcccggcgttcgcccgatcccgtccttcgacgacatgccgctgcaccagaacctgctgcgtggcatctactcgtacgggttcgagaagccgtccagcatccagcagcgcgcgatagcccccttcacgcgcggcggcgacatcatcgcgcaggcccagtccggtaccggcaagacgggtgccttctccatcggtctgctgcagcgcctggacttccgccacaacctgatccagggcctcgtgctctcccccactcgcgagctggccctgcagacggcggaggtgatcagccgcatcggtgagttcctgtcgaacagctccaagttctgcgagacctttgtcggcggcacgcgcgtgcaggatgacctgcgcaagctgcaggccggcgtcatcgttgccgtgggcacgccgggccgcgtgtccgacgtgatcaagcgtggcgcgctgcgcacagagtcgctgcgcgtgctggtgctcgacgaggctgatgagatgctgtctcagggcttcgcggaccagatttacgagatcttccgcttcctgccgaaggacatccaggtcgcgctcttctccgccacgatgccggaggaggtactggagctgacgaagaagttcatgcgcgac

SEQ ID NO: 12 - protein, 932 aa, Artificial Sequence
  Synthetic Construct
MSCGNAKINSPAPSFEEVALMPNGSFKKISLSSYKGKWVVLFFYPLDFTFVCPTEVIAFSDSVSRFNELNCEVLACSIDSEYAHLQWTLQDRKKGGLGTMAIPMLADKTKSIARSYGVLEESQGVAYRGLFIIDPHGMLRQITVNDMPVGRSVEEVLRLLEAFQFVEKHGEVCPANWKKGAPTMKPEPNASVEGYFSKQGSMQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQEDEFMAQNDKIAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRD

SEQ ID NO: 13 - DNA, 2766 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagactagtatgcaggcctacacacaactggagaagctctgccagaaggtgtacagattggcgcaccttctgtctctcggcgcttgggattccaagactatgatgccctcaaagggcgcagctgcccgcggtgccgccctcggcgagctctacggactcatcgctgagatgatcaccagcccgagcacgaaggcgctgctggacgaagcagagacggccaaggccgagctcactactgtccagcaggcgaacttgcgcgagctccgccgcatgtacacctctcaagcagcgctaccgaccgagttcagtgtgctcaagaccaagctttcgtcaactactccgcttatctgggttaagtgccgcagcaacaacgactttgcgactttcctgccggcgctgaaggagatgattgcgcttgcgcgcagggaggcgcagtatcgctctactgcgacgggcaagcctctgtacgaggccctgttcaaccagtacgagagcggcatgacgctggagacgctggaaaaaatcttgctcgatgtgaagtcgtggctgccggagctgctgcagaagatcctggctgcacagagggacgcggggctggaggtggttgcgcctgaggcgccctttcccaaggacaagcaggaggctcttagccgccacctcatggaggtgtggggcttcgacttcgagtcaggtcggctggacgtctctgagcacccgtttatgggcatggtaaaggaagactcgcgcatcactaccgcctacgacctgcaggacttcaccaaggggctcttcgcgacgatccacgagacgggccactccaagtacgagacgaactgcggcccggtggagatgcgcggccagccggtgtgcgaggcacgctcgatgacgatccacgagagccagtcgcgctttgccgaggttgtgattggccactccagcgccttcttggagttcctcgttccactgctgaaggaatacctcggtgatcagcccgcattctctcgggagaacgtgcggctgatgaaccagacggtgaagcctggcttcatccggatccgggcggatgaggtgtgctacccgctgcacatcttgctgcgctacgagatagagcgtgcactcatcgagggcacgatggaggcagaagacatccctcgcgtgtggaacgagaagatgaaggcatacctgggcctggagacggagggccgcgacgagattggctgcctgcaggacattcactggtcgatgggcgcctttggctacttcccgacgtactcgcttggctccatgttcgcggcgcagctgatggcgacgatcaagaatgagctcggtgaggatacagtggacaagtgcatccgcactggccagatggagccgatctttgagaagcagagggagaagatctggagccagggatgcctctacaacacggaagacctgattgtcaaggcgaccggcgaagcgctgaaccccaagtactttcgcgagtacctggaacgccgctacctgcgccaggaggacgctagcatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacggatatcagcgcctccgctgagccgcacaaggcggccgttgacgtcggcccgctgagcgttggcccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgctgagcgttggcccgcagagcgttggcccgctgagcgttggcagccagagcgtcggcccgctgagcgttggtccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgcagagcgttggcccgctgagcgttggcagccagagcgtcggcccgctgagcgttggtccgcagagcgtcggcccgctgagcgttggcccgcagagcgtcggcccgctgagcgttggcccgcagagcgtcggcccgctgagcgttggtccgcagagcgttggcccgctgagcgttggcccgcagagcgttgacgttagcccggtgagc

SEQ ID NO: 14 - protein, 922 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKTSMQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQEDASMASSRSAPRKASHAHKSHRKPKRSWNVYVGRSLKAINAQMSMSHRTDISASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 15 - DNA, 2199 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagactagtatgccgcgcaagattattctcgattgtgatcccgggatcgatgatgccgtggccatctttctcgcccacggcaacccggaggtcgagctgctggccattacgacggtggtgggcaaccagaccctggagaaggtgacccggaacgcgcggctggtagctgacgtagccggcatcgttggtgtgcccgtcgcggctggttgcaccaagcccctcgtgcgcggtgtgcggaatgcctctcagattcatggcgaaaccggcatgggtaacgtctcctacccaccagagttcaagacaaagttggacggccgtcatgcagtgcagctgatcatcgaccttatcatgtcgcacgagccgaagacgatcacgcttgtgcctacgggtggcctgacgaacattgcgatggctgtccgtcttgagccgcgcatcgtggaccgtgtgaaggaggtggttctgatgggtggcggctaccatactggtaatgcgtcccctgtagcggagttcaacgtcttcgtcgacccggaggcggcgcacattgtgttcaacgagagctggaacgtaacgatggtggggctggacctaacgcaccaggcactcgccacgccggcggtccagaagcgagtgaaggaggtgggcacgaagccggctgccttcatgctgcagattttggacttttacacgaaggtgtacgaaaaggagcgcaacacgtacgcgacggtgcacgatccctgcgctgtggcgtacgtgattgaccccaccgtgatgacgacggagcaagtgccagtggacatcgagctcaatggggcactgacgactgggatgacggtcgcggacttccgctacccacggccaaagcactgccacacgcaggtggctgtgaagctggacttcgacaagttttggtgcctcgtgattgacgcactcaagcgcatcggcgatcctcaagctagcatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacggatatcagcgcctccgctgagccgcacaaggcggccgttgacgtcggcccgctgagcgttggcccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgctgagcgttggcccgcagagcgttggcccgctgagcgttggcagccagagcgtcggcccgctgagcgttggtccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgcagagcgttggcccgctgagcgttggcagccagagcgtcggcccgctgagcgttggtccgcagagcgtcggcccgctgagcgttggcccgcagagcgtcggcccgctgagcgttggcccgcagagcgtcggcccgctgagcgttggtccgcagagcgttggcccgctgagcgttggcccgcagagcgttgacgttagcccggtgagc

SEQ ID NO: 16 - protein, 733 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKTSMPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQASMASSRSAPRKASHAHKSHRKPKRSWNVYVGRSLKAINAQMSMSHRTDISASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 17 - DNA, 2127 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagactagttcggcggtcggcaacatcgagtcgcagtgggcccgtgccggccacggcttggtgagcctgtcggagcagcagctggtgagctgcgatgacaaagacaatggctgcaacggcgggctgatgctgcaggcgttcgagtggctgctgcgacacatgtacgggatcgtgttcacggagaagagctacccctacacgtccggcaacggtgatgtggccgagtgcttgaacagcagtaaactcgttcccggcgcgcaaatcgacggctacgtgatgatcccgagcaacgaaacggttatggctgcgtggcttgcggagaatggccccatcgcgattgcggtcgacgccagctccttcatgtcttaccagagcggcgtgctgaccagctgcgctggcgatgcactgaaccacggcgtgctgctcgtcgggtacaacaagaccggtggggttccgtactgggtgatcaagaactcgtggggtgaggactggggcgagaagggctacgtgcgcgtggtcatggggctgaacgcgtgcctgctcagtgaataccccgtgtccgcgcatgtgccgcggagtctcacccctggcccgggcacggagagcgaggagcgcgcccctaaacgggtgacggtggagcagatgatgtgcaccgatatgtactgcagggaggggtgcaagaagagtcttctcaccgcgaacgtgtgctacaagaacgggggaggcggctcctctatgacgaagtgcggtccgcagaaggtgctgatgtgctcgtactcgaaccctcattgctttggtcctgggctgtgcctcgagactcctgatggcaagtgcgcgccgtacttcttgggctcgatcatgaacacctgccagtacacggctagcatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacggatatcagcgcctccgctgagccgcacaaggcggccgttgacgtcggcccgctgagcgttggcccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgctgagcgttggcccgcagagcgttggcccgctgagcgttggcagccagagcgtcggcccgctgagcgttggtccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgcagagcgttggcccgctgagcgttggcagccagagcgtcggcccgctgagcgttggtccgcagagcgtcggcccgctgagcgttggcccgcagagcgtcggcccgctgagcgttggcccgcagagcgtcggcccgctgagcgttggtccgcagagcgttggcccgctgagcgttggcccgcagagcgttgacgttagcccggtgagc

SEQ ID NO: 18 - protein, 709 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKTSSAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYTASMASSRSAPRKASHAHKSHRKPKRSWNVYVGRSLKAINAQMSMSHRTDISASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 19 - DNA, 2931 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagactagtatgccgcgcaagattattctcgattgtgatcccgggatcgatgatgccgtggccatctttctcgcccacggcaacccggaggtcgagctgctggccattacgacggtggtgggcaaccagaccctggagaaggtgacccggaacgcgcggctggtagctgacgtagccggcatcgttggtgtgcccgtcgcggctggttgcaccaagcccctcgtgcgcggtgtgcggaatgcctctcagattcatggcgaaaccggcatgggtaacgtctcctacccaccagagttcaagacaaagttggacggccgtcatgcagtgcagctgatcatcgaccttatcatgtcgcacgagccgaagacgatcacgcttgtgcctacgggtggcctgacgaacattgcgatggctgtccgtcttgagccgcgcatcgtggaccgtgtgaaggaggtggttctgatgggtggcggctaccatactggtaatgcgtcccctgtagcggagttcaacgtcttcgtcgacccggaggcggcgcacattgtgttcaacgagagctggaacgtaacgatggtggggctggacctaacgcaccaggcactcgccacgccggcggtccagaagcgagtgaaggaggtgggcacgaagccggctgccttcatgctgcagattttggacttttacacgaaggtgtacgaaaaggagcgcaacacgtacgcgacggtgcacgatccctgcgctgtggcgtacgtgattgaccccaccgtgatgacgacggagcaagtgccagtggacatcgagctcaatggggcactgacgactgggatgacggtcgcggacttccgctacccacggccaaagcactgccacacgcaggtggctgtgaagctggacttcgacaagttttggtgcctcgtgattgacgcactcaagcgcatcggcgatcctcaagctagctcggcggtcggcaacatcgagtcgcagtgggcccgtgccggccacggcttggtgagcctgtcggagcagcagctggtgagctgcgatgacaaagacaatggctgcaacggcgggctgatgctgcaggcgttcgagtggctgctgcgacacatgtacgggatcgtgttcacggagaagagctacccctacacgtccggcaacggtgatgtggccgagtgcttgaacagcagtaaactcgttcccggcgcgcaaatcgacggctacgtgatgatcccgagcaacgaaacggttatggctgcgtggcttgcggagaatggccccatcgcgattgcggtcgacgccagctccttcatgtcttaccagagcggcgtgctgaccagctgcgctggcgatgcactgaaccacggcgtgctgctcgtcgggtacaacaagaccggtggggttccgtactgggtgatcaagaactcgtggggtgaggactggggcgagaagggctacgtgcgcgtggtcatggggctgaacgcgtgcctgctcagtgaataccccgtgtccgcgcatgtgccgcggagtctcacccctggcccgggcacggagagcgaggagcgcgcccctaaacgggtgacggtggagcagatgatgtgcaccgatatgtactgcagggaggggtgcaagaagagtcttctcaccgcgaacgtgtgctacaagaacgggggaggcggctcctctatgacgaagtgcggtccgcagaaggtgctgatgtgctcgtactcgaaccctcattgctttggtcctgggctgtgcctcgagactcctgatggcaagtgcgcgccgtacttcttgggctcgatcatgaacacctgccagtacacggatatcagcgcctccgctgagccgcacaaggcggccgttgacgtcggcccgctgagcgttggcccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgctgagcgttggcccgcagagcgttggcccgctgagcgttggcagccagagcgtcggcccgctgagcgttggtccgcagagcgtcggcccgctgagcgttggcccgcaggcggttggcccgctgagcgttggcccgcagagcgtcggcccgctgagcgttggcccgcaggcggttggcccgctgagcgttggcccgcagagcgttggcccgctgagcgttggcccgcagagcgttggcccgctgagcgttggcagccagagcgtcggcccgctgagcgttggtccgcagagcgtcggcccgctgagcgttggcccgcagagcgtcggcccgctgagcgttggcccgcagagcgtcggcccgctgagcgttggtccgcagagcgttggcccgctgagcgttggcccgcagagcgttgacgttagcccggtgagc

SEQ ID NO: 20 - protein, 977 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKTSMPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQASSAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYTDISASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 21 - protein, 152 aa, Unknown
  From Leishmania infantum or Leishmania donovani
KDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKK

SEQ ID NO: 22 - protein, 152 aa, Leishmania major
KDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQSQGEQQQQQNSEEKK

SEQ ID NO: 23 - protein, 157 aa, Leishmania mexicana
KDKATGKTQNITITANGGLSKEQIEQMIRDSEQHAEADRVKRELVEVRNNAETQLTTAERQLSEWKYVSDAEKENVRTLVAELRKAMENPNVAKDDLSAATDKLQKAVMECGRTEYQQAAAANSGSTSNSGEQQQQQQQSQGEQQQQQQQQQQAEER

SEQ ID NO: 24 - protein, 152 aa, Leishmania braziliensis
KDKATGKTQNITITAHGGLSKEQIEQMVRDSEQHAEADRVKRELVEARNNAETQLTTAERQLGEWKYVSDAEKENVKTHVAELRKAMENPNVAKDDLAAATDKLQKAVMECGRTEYQQAAAANSGSSSNSGEQQQQQQQQGDQQQQQSSEKN

SEQ ID NO: 25 - protein, 503 aa, Leishmania donovani
MQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQED

SEQ ID NO: 26 - protein, 503 aa, Leishmania infantum
MQAYTQLEKLCQKVYRLAHLLSLGAWDSKTMMPSKGAAARGAALGELYGLIAEMITSPSTKALLDEAEAAKAELTTVQQANLRELRRMYTSQAALPTEFSVLKTKLSSTTPLIWAKCRSNNDFATFLPALKEMIALARREAQYRSTATGKPLYEALFNQYESGMTLETLEKILLDVKSWLPELLQKILAAQRDAGLEVVAPEAPFPKDKQEALSRHLMEVWGFDFESGRLDVSEHPFMGMVKEDSRITTAYDLQDFTKGLFATIHETGHSKYETNCGPVEMRGQPVCEARSMTIHESQSRFAEVVIGHSSAFLEFLVPLLKEYLGDQPAFSRENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMATIKNELGEDTVDKCIRTGQMEPIFEKQREKIWSQGCLYNTEDLIVKATGEALNPKYFREYLERRYLRQED

SEQ ID NO: 27 - protein, 503 aa, Leishmania major
MQAYTQLEKLCHKVHRLTHLLSLGAWDAKTMMPSKGAAARGAALGELHGLITEMITSPSTKALLDEAETAKAELTTVQQANLRELRRIYASQAALPTELRVLKTKLSATTPLIWAKCRSNNDFATFLPALKEMIALARREAQYRSAATGKPLYEALFNQYESGMTLETLEKILLDVKSWLPELLQKILAAQRDAGLEVVAPEAPFPKDKQEALSRHLMEVWGFDFESGRLDVSEHPFTGMVKEDSRITTAYDLQDFAKGLFATIHETGHSKYETNCGPMEMRGQPVCEARSMTIHESQSRFAEVVIGHSSAFLEFLTPLLKEYFGDQPAFSLENVRLMNQTVKPGFIRIRADEVCYPLHILLRYEIERALIEGTMEAEDIPRVWNEKMKAYLGLETEGRDEIGCLQDIHWSMGAFGYFPTYSLGSMFAAQLMVTIKNELGEDTVDKCIRTGQMEPIFEKQREKIWSQGCLYDTEDLILKATGEALNPKHFREYLERRYLRQEG

SEQ ID NO: 28 - protein, 503 aa, Leishmania mexicana
MQAYSQLEKLCQKVYRLEHLLSLGAWDAKTMMPSKGAAARGAALGELYGLIAEMITSPSTKTLLDEAETAKAELTTVQQANLRELRRMYTSQAALPTEFSVLKAKLSSTTPLIWAKCRSNNDFVTFLPALKEMIALARREAQYRSTATGKPLYEALFNQYESGMTLETLEKNLLDVKSWLPELLQKILAAQKDAGREAVAPEAPFPKDKQEALSRHLMKVWGFDFESGRLDVSEHPFMGMVKEDSRITTAYDLQDFTKGLFATIHETGHSKYETNCGPMEMRGQPVCEARSMTIHESQSRFAEVVIGHSSAFLEFLVPLLKEYLGDQPTLSLENVRLMNQTVKPGFIRIRADEVCYPLHILLRYEIERALIEGTMEAEDIPRVWNEKMKAYLGLETEGRDEIGCLQDIHWPMGAFGYFPTYSLGSMFAVQLMATIKKELGEDTVDKCIRTGQMEPIFQKQREKIWSQGCLYNTEDLIVKATGETLNPKHFREYLERRYLRQED

SEQ ID NO: 29 - protein, 503 aa, Leishmania braziliensis
MQAYKQLEQLSQKLHNLSHFLYLGKWDSETMMPSKGSAARGAAIGELHGLIAELMTAPSTKTLLDEAEGVKTELTKTQQANLREFRRMYSAQAALPNDFSMLKARLSTTVPLIWAECRRNNDFATFVPTLKEVITVARKEAQYRSAATGKPLYEALFNQYECGMTLETVDSIFSDVKSWLPELLQKILTLQKAEGLEARAPEAPFPKDKQDALGRHLMKVWGFDFESGRLDVSAHPFTGMVKEDSRITTNYDLEDFTKALFATIHETGHSKYETNCGPMDMRGQPVCNARSLMIHESQSRFAEVVIGRSSAFPEFLAPLLKEHLGEQPAFSLENVRLMSQRVRPGFIRIFADEVCYPLHVLLRYEIERALIEGTMEVEDIPRVWNEKMKAYLGLETEGRDDIGCLQDTHWAMGAFGYFPTYTLGTMFAVQLMYTIKKELGESTVDKCIRTGQMEPIFAKQKEKIWDQGCLYETEELMIKATGETLNPKYFREYLERRYLRHED

SEQ ID NO: 30 - protein, 290 aa, Leishmania infantum
SAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYT

SEQ ID NO: 31 - protein, 46 aa, Leishmania infantum
MASSRSAPRKASHAHKSHRKPKRSWNVYVGRSLKAINAQMSMSHRT

SEQ ID NO: 32 - protein, 214 aa, Leishmania donovani
SASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 33 - protein, 190 aa, Leishmania infantum
SIIKEDDAVGCYMTVTLVDDTKVEGTIFTYNPKEGIIVLLSLRDDQTNMKLIRTPYIKEFSISHAEEGTHLPPALDSFNELPSMHAGRDKSIFKHASTQLKNAEANREKHFNSVTTDTPIATLDAYLKLLRLYPFIEWNSDEGVIQVSDTVIVVGDPDWRTPKAMLVDGAPEKDRPLVDRLQVALGNGKK

SEQ ID NO: 34 - protein, 188 aa, Leishmania major
MQAYTQLEKLCQKVYRLAHLLSLGAWDSKTMMPSKGAAARGAALGELYGLIAEMITSPSTKALLDEAETAKAELTTVQQANLRELRRMYTSQAALPTEFSVLKTKLSSTTPLIWVKCRSNNDFATFLPALKEMIALARREAQYRSTATGKPLYEALFNQYESGMTLETLEKILLDVKSWLPELLQKIL

SEQ ID NO: 35 - protein, 226 aa, Leishmania major
MAQNDKIAPQDQDSFLDDQPGVRPIPSFDDMPLHQNLLRGIYSYGFEKPSSIQQRAIAPFTRGGDIIAQAQSGTGKTGAFSIGLLQRLDFRHNLIQGLVLSPTRELALQTAEVISRIGEFLSNSSKFCETFVGGTRVQDDLRKLQAGVIVAVGTPGRVSDVIKRGALRTESLRVLVLDEADEMLSQGFADQIYEIFRFLPKDIQVALFSATMPEEVLELTKKFMRD

SEQ ID NO: 36 - protein, 314 aa, Unknown
  From Leishmania infantum or Leishmania donovani
MPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQ

SEQ ID NO: 37 - protein, 236 aa, Leishmania donovani
MKIRSVRPLVVLLVSVAAVLALSASAEPHKAAVDVGPLSVGPQSVGPLSVGPQAVGPLSVGPQSVGPLSVGPQAVGPLSVGPQSVGPLSVGPLSVGPQSVGPLSVGSQSVGPLSVGPQSVGPLSVGPQAVGPLSVGPQSVGPLSVGPQAVGPLSVGPQSVGPLSVGPQSVGPLSVGSQSVGPLSVGPQSVGPLSVGPQSVGPLSVGPQSVGPLSVGPQSVGPLSVGPQSVDVSPVS

SEQ ID NO: 38 - protein, 490 aa, Leishmania infantum
MRDAHTRTPTEKKTRSSSLSFFEQTPLNRLLTPLSSFSAMREAICIHIGQAGCQVGNACWELFCLEHGIQPDGSMPSDKCIGVEDDAFNTFFSETGAGKHVPRCIFLDLEPTVVDEVRTGTYRQLFNPEQLVSGKEDAANNYARGHYTIGKEIVDLALDRIRKLADNCTGLQGFMVFHAVGGGTGSGLGALLLERLSVDYGKKSKLGYTVYPSPQVSTAVVEPYNCVLSTHSLLEHTDVATMLDNEAIYDLTRRSLDIERPSYTNVNRLIGQVVSSLTASLRFDGALNVDLTEFQTNLVPYPRIHFVLTSYAPVVSAEKAYHEQLSVADITNSVFEPAGMLTKCDPRHGKYMSCCLMYRGDVVPKDVNAAIATIKTKRTIQFVDWCPTGFKCGINYQPPTVVPGGDLAKVQRAVCMIANSTAIAEVFARIDHKFDLMYSKRAFVHWYVGEGMEEGEFSEAREDLAALEKDYEEVGAESADDMGEEDVEEY

SEQ ID NO: 39 - protein, 322 aa, Leishmania infantum
MVNVCVVGAAGGIGQSLSLLLVRQLPYGSTLSLFDVVGAAGVAADLSHVDNAGVQVKFAAGKIGQKRDPALAELAKGVDVFVMVAGVPRKPGMTRDDLFKINAGIILDLVLTCASSSPKAVFCIVTNPVNSTVVIAAEALKSLGVYDRNRLLGVSLLDGLRATCFINEARKPLVVTQVPVVGGHSDATIVPLFHQLLGPLPEQATLDKIVKRVQVAGTEVVKAKAGRGSATLSMAEAGARFTLKVVEGLTGTGKPLVYAYVDTDGQHETPFLAIPVVLGVNGIEKRLPIGPLHSTEETLLKAALPVIKKNIVKGSEFARSHL

SEQ ID NO: 40 - DNA, 2271 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgccgcgcaagattattctcgattgtgatcccgggatcgatgatgccgtggccatctttctcgcccacggcaacccggaggtcgagctgctggccattacgacggtggtgggcaaccagaccctggagaaggtgacccggaacgcgcggctggtagctgacgtagccggcatcgttggtgtgcccgtcgcggctggttgcaccaagcccctcgtgcgcggtgtgcggaatgcctctcagattcatggcgaaaccggcatgggtaacgtctcctacccaccagagttcaagacaaagttggacggccgtcatgcagtgcagctgatcatcgaccttatcatgtcgcacgagccgaagacgatcacgcttgtgcctacgggtggcctgacgaacattgcgatggctgtccgtcttgagccgcgcatcgtggaccgtgtgaaggaggtggttctgatgggtggcggctaccatactggtaatgcgtcccctgtagcggagttcaacgtcttcgtcgacccggaggcggcgcacattgtgttcaacgagagctggaacgtaacgatggtggggctggacctaacgcaccaggcactcgccacgccggcggtccagaagcgagtgaaggaggtgggcacgaagccggctgccttcatgctgcagattttggacttttacacgaaggtgtacgaaaaggagcgcaacacgtacgcgacggtgcacgatccctgcgctgtggcgtacgtgattgaccccaccgtgatgacgacggagcaagtgccagtggacatcgagctcaatggggcactgacgactgggatgacggtcgcggacttccgctacccacggccaaagcactgccacacgcaggtggctgtgaagctggacttcgacaagttttggtgcctcgtgattgacgcactcaagcgcatcggcgatcctcaatcggcggtcggcaacatcgagtcgcagtgggcccgtgccggccacggcttggtgagcctgtcggagcagcagctggtgagctgcgatgacaaagacaatggctgcaacggcgggctgatgctgcaggcgttcgagtggctgctgcgacacatgtacgggatcgtgttcacggagaagagctacccctacacgtccggcaacggtgatgtggccgagtgcttgaacagcagtaaactcgttcccggcgcgcaaatcgacggctacgtgatgatcccgagcaacgaaacggttatggctgcgtggcttgcggagaatggccccatcgcgattgcggtcgacgccagctccttcatgtcttaccagagcggcgtgctgaccagctgcgctggcgatgcactgaaccacggcgtgctgctcgtcgggtacaacaagaccggtggggttccgtactgggtgatcaagaactcgtggggtgaggactggggcgagaagggctacgtgcgcgtggtcatggggctgaacgcgtgcctgctcagtgaataccccgtgtccgcgcatgtgccgcggagtctcacccctggcccgggcacggagagcgaggagcgcgcccctaaacgggtgacggtggagcagatgatgtgcaccgatatgtactgcagggaggggtgcaagaagagtcttctcaccgcgaacgtgtgctacaagaacgggggaggcggctcctctatgacgaagtgcggtccgcagaaggtgctgatgtgctcgtactcgaaccctcattgctttggtcctgggctgtgcctcgagactcctgatggcaagtgcgcgccgtacttcttgggctcgatcatgaacacctgccagtacacg

SEQ ID NO: 41 - protein, 757 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQSAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYT

SEQ ID NO: 42 - DNA, 2604 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgccgcgcaagattattctcgattgtgatcccgggatcgatgatgccgtggccatctttctcgcccacggcaacccggaggtcgagctgctggccattacgacggtggtgggcaaccagaccctggagaaggtgacccggaacgcgcggctggtagctgacgtagccggcatcgttggtgtgcccgtcgcggctggttgcaccaagcccctcgtgcgcggtgtgcggaatgcctctcagattcatggcgaaaccggcatgggtaacgtctcctacccaccagagttcaagacaaagttggacggccgtcatgcagtgcagctgatcatcgaccttatcatgtcgcacgagccgaagacgatcacgcttgtgcctacgggtggcctgacgaacattgcgatggctgtccgtcttgagccgcgcatcgtggaccgtgtgaaggaggtggttctgatgggtggcggctaccatactggtaatgcgtcccctgtagcggagttcaacgtcttcgtcgacccggaggcggcgcacattgtgttcaacgagagctggaacgtaacgatggtggggctggacctaacgcaccaggcactcgccacgccggcggtccagaagcgagtgaaggaggtgggcacgaagccggctgccttcatgctgcagattttggacttttacacgaaggtgtacgaaaaggagcgcaacacgtacgcgacggtgcacgatccctgcgctgtggcgtacgtgattgaccccaccgtgatgacgacggagcaagtgccagtggacatcgagctcaatggggcactgacgactgggatgacggtcgcggacttccgctacccacggccaaagcactgccacacgcaggtggctgtgaagctggacttcgacaagttttggtgcctcgtgattgacgcactcaagcgcatcggcgatcctcaatcggcggtcggcaacatcgagtcgcagtgggcccgtgccggccacggcttggtgagcctgtcggagcagcagctggtgagctgcgatgacaaagacaatggctgcaacggcgggctgatgctgcaggcgttcgagtggctgctgcgacacatgtacgggatcgtgttcacggagaagagctacccctacacgtccggcaacggtgatgtggccgagtgcttgaacagcagtaaactcgttcccggcgcgcaaatcgacggctacgtgatgatcccgagcaacgaaacggttatggctgcgtggcttgcggagaatggccccatcgcgattgcggtcgacgccagctccttcatgtcttaccagagcggcgtgctgaccagctgcgctggcgatgcactgaaccacggcgtgctgctcgtcgggtacaacaagaccggtggggttccgtactgggtgatcaagaactcgtggggtgaggactggggcgagaagggctacgtgcgcgtggtcatggggctgaacgcgtgcctgctcagtgaataccccgtgtccgcgcatgtgccgcggagtctcacccctggcccgggcacggagagcgaggagcgcgcccctaaacgggtgacggtggagcagatgatgtgcaccgatatgtactgcagggaggggtgcaagaagagtcttctcaccgcgaacgtgtgctacaagaacgggggaggcggctcctctatgacgaagtgcggtccgcagaaggtgctgatgtgctcgtactcgaaccctcattgctttggtcctgggctgtgcctcgagactcctgatggcaagtgcgcgccgtacttcttgggctcgatcatgaacacctgccagtacacgatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacgatgagcatcgtgaactcgtacgtgaacgacgtgatggagcgcatctgcatggaggccgcgtcgatcgttcgcgcgaacaagaagcgcacgttgggtgcgcgcgaggtgcagacggcggtgcgcattgtgctgccggcggagctcgcgaagcacgccatggctgagggcacgaaggccgtgtcgagcgcgtcggct

SEQ ID NO: 43 - protein, 868 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMPRKIILDCDPGIDDAVAIFLAHGNPEVELLAITTVVGNQTLEKVTRNARLVADVAGIVGVPVAAGCTKPLVRGVRNASQIHGETGMGNVSYPPEFKTKLDGRHAVQLIIDLIMSHEPKTITLVPTGGLTNIAMAVRLEPRIVDRVKEVVLMGGGYHTGNASPVAEFNVFVDPEAAHIVFNESWNVTMVGLDLTHQALATPAVQKRVKEVGTKPAAFMLQILDFYTKVYEKERNTYATVHDPCAVAYVIDPTVMTTEQVPVDIELNGALTTGMTVADFRYPRPKHCHTQVAVKLDFDKFWCLVIDALKRIGDPQSAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYTMASSRSAPRKASHAHKSHRKPKRSWNVYVGRSLKAINAQMSMSHRTMSIVNSYVNDVMERICMEAASIVRANKKRTLGAREVQTAVRIVLPAELAKHAMAEGTKAVSSASA

SEQ ID NO: 44 - DNA, 2629 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatggtaaacgtgtgcgttgttggggctgccggcggcatcgggcagtcgctgtcgcttctgctggtgcgccagctgccgtacgggagcacgttgtcgttgttcgacgttgtgggcgctgccggcgttgcagcggacctgtcgcacgtggacaacgccggtgtgcaggtgaagttcgcggcgggcaagataggccagaagcgcgaccctgcgctagcggagcttgcgaagggcgtggatgtgtttgtgatggtggctggcgtgccacgcaagccgggcatgacgcgcgacgaccttttcaaaatcaacgccggaatcatcctggaccttgtgctgacgtgcgcatcgtcgagcccaaaggcggtgttctgcattgtgacgaaccctgtgaacagcacggtcgtgatcgcggcagaggcgctgaagagcctcggcgtatacgacagaaaccggctgcttggcgtgtcgctgctagacgggctgcgcgcgacgtgcttcatcaacgaggcgcgcaagcctttggtcgtgacgcaggtgccagttgttggcgggcacagcgacgcaacgattgttccgttgttccaccagctgctggggccgttgccggagcaggcgacgctggacaagatcgtgaagcgcgtgcaggttgcaggcacagaggtggtgaaggcgaaggccgggcgcgggtctgcgacgctgtcgatggcggaggctggcgcgcggttcacgctgaaggttgtggagggcctgaccggcacgggtaaaccgctggtgtacgcatacgtggacacagacgggcagcacgagacgccgttcctcgcgatccccgtggtgcttggcgtgaatggaatcgagaagcgcctgccaatcggtccgctgcactcgacagaggaaacgctgctgaaggcggcactgccggtgatcaagaagaatatcgtgaagggcagcgagttcgcgcgctcacacctgtcggcggtcggcaacatcgagtcgcagtgggcccgtgccggccacggcttggtgagcctgtcggagcagcagctggtgagctgcgatgacaaagacaatggctgcaacggcgggctgatgctgcaggcgttcgagtggctgctgcgacacatgtacgggatcgtgttcacggagaagagctacccctacacgtccggcaacggtgatgtggccgagtgcttgaacagcagtaaactcgttcccggcgcgcaaatcgacggctacgtgatgatcccgagcaacgaaacggttatggctgcgtggcttgcggagaatggccccatcgcgattgcggtcgacgccagctccttcatgtcttaccagagcggcgtgctgaccagctgcgctggcgatgcactgaaccacggcgtgctgctcgtcgggtacaacaagaccggtggggttccgtactgggtgatcaagaactcgtggggtgaggactggggcgagaagggctacgtgcgcgtggtcatggggctgaacgcgtgcctgctcagtgaataccccgtgtccgcgcatgtgccgcggagtctcacccctggcccgggcacggagagcgaggagcgcgcccctaaacgggtgacggtggagcagatgatgtgcaccgatatgtactgcagggaggggtgcaagaagagtcttctcaccgcgaacgtgtgctacaagaacgggggaggcggctcctctatgacgaagtgcggtccgcagaaggtgctgatgtgctcgtactcgaaccctcattgctttggtcctgggctgtgcctcgagactcctgatggcaagtgcgcgccgtacttcttgggctcgatcatgaacacctgccagtacacgatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacgatgagcatcgtgaactcgtacgtgaacgacgtgatggagcgcatctgcatggaggccgcgtcgatcgttcgcgcgaacaagaagcgcacgttgggtgcgcgcgaggtgcagacggcggtgcgcattgtgctgccggcggagctcgcgaagcacgccatggctgagggcacgaaggccgtgtcgagcgcgtcggctt

SEQ ID NO: 45 - protein, 876 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMVNVCVVGAAGGIGQSLSLLLVRQLPYGSTLSLFDVVGAAGVAADLSHVDNAGVQVKFAAGKIGQKRDPALAELAKGVDVFVMVAGVPRKPGMTRDDLFKINAGIILDLVLTCASSSPKAVFCIVTNPVNSTVVIAAEALKSLGVYDRNRLLGVSLLDGLRATCFINEARKPLVVTQVPVVGGHSDATIVPLFHQLLGPLPEQATLDKIVKRVQVAGTEVVKAKAGRGSATLSMAEAGARFTLKVVEGLTGTGKPLVYAYVDTDGQHETPFLAIPVVLGVNGIEKRLPIGPLHSTEETLLKAALPVIKKNIVKGSEFARSHLSAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYTMASSRSAPRKASHAHKSHRKPKRSWNVYVGRSLKAINAQMSMSHRTMSIVNSYVNDVMERICMEAASIVRANKKRTLGAREVQTAVRIVLPAELAKHAMAEGTKAVSSASA

SEQ ID NO: 46 - DNA, 3228 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatggtaaacgtgtgcgttgttggggctgccggcggcatcgggcagtcgctgtcgcttctgctggtgcgccagctgccgtacgggagcacgttgtcgttgttcgacgttgtgggcgctgccggcgttgcagcggacctgtcgcacgtggacaacgccggtgtgcaggtgaagttcgcggcgggcaagataggccagaagcgcgaccctgcgctagcggagcttgcgaagggcgtggatgtgtttgtgatggtggctggcgtgccacgcaagccgggcatgacgcgcgacgaccttttcaaaatcaacgccggaatcatcctggaccttgtgctgacgtgcgcatcgtcgagcccaaaggcggtgttctgcattgtgacgaaccctgtgaacagcacggtcgtgatcgcggcagaggcgctgaagagcctcggcgtatacgacagaaaccggctgcttggcgtgtcgctgctagacgggctgcgcgcgacgtgcttcatcaacgaggcgcgcaagcctttggtcgtgacgcaggtgccagttgttggcgggcacagcgacgcaacgattgttccgttgttccaccagctgctggggccgttgccggagcaggcgacgctggacaagatcgtgaagcgcgtgcaggttgcaggcacagaggtggtgaaggcgaaggccgggcgcgggtctgcgacgctgtcgatggcggaggctggcgcgcggttcacgctgaaggttgtggagggcctgaccggcacgggtaaaccgctggtgtacgcatacgtggacacagacgggcagcacgagacgccgttcctcgcgatccccgtggtgcttggcgtgaatggaatcgagaagcgcctgccaatcggtccgctgcactcgacagaggaaacgctgctgaaggcggcactgccggtgatcaagaagaatatcgtgaagggcagcgagttcgcgcgctcacacctgatgcgcgatgcacacacgcgcacgcccaccgaaaaaaaaacgcgcagctcttcgctctcgttcttcgaacaaacacctttaaaccgccttctaacccctctttcttctttttcagccatgcgtgaggctatctgcatccacatcggccaggccggctgccaggtcggtaacgcgtgctgggagctgttctgccttgagcacggcatccagcctgatggctccatgccctctgacaagtgcatcggtgttgaggatgacgcgttcaacacgttcttctcggagactggtgctggcaagcacgttcctcgctgcatcttcctggacctcgagcctacggtcgtggatgaggtgcgcaccggcacgtaccgccagctgttcaaccccgagcagctggtgtccggcaaggaggatgcggcgaacaactacgctcgtggccactacaccatcggcaaggagatcgtcgaccttgcgctggaccgcattcgcaagctggcggacaactgcacgggtctccagggctttatggtgttccacgctgtgggtggcggcaccggctctggcctcggtgcgctgctgctggagcgcctgtctgtggactacggcaagaagtccaagcttggctacaccgtgtacccgagcccgcaggtgtcgactgccgtcgtggagccgtacaactgcgtgctgtcgacgcactcgctgctcgagcacaccgatgttgcgacgatgctcgacaatgaggccatctacgacctcactcgtcgttctctcgacattgagcgcccgtcgtacacgaacgtgaaccgcctgatcggccaggtggtgtcgtctctgacggcgtcgctgcgcttcgatggtgcgctgaacgtggacctgacggagttccagacgaaccttgtgccgtacccgcgcatccacttcgtgctgacgagctatgctccggtggtgtctgccgagaaggcgtaccacgagcagctgtccgtcgcggacatcacgaactcggtgtttgagcctgctggcatgctgacgaagtgcgatcctcgccacggcaagtacatgtcgtgctgcctcatgtaccgcggtgatgtcgtgccgaaggatgtcaacgccgcgattgcgacgatcaagacgaagcggacaattcagttcgtggactggtgtccgaccggcttcaagtgcggcatcaactaccagccgccgaccgttgtgcccggcggtgacctcgcgaaggtgcagcgcgccgtgtgcatgattgccaactcgaccgcgatcgctgaggtgtttgcccgcatcgaccacaagttcgacctgatgtacagcaagcgcgcgttcgtgcactggtacgtgggtgagggcatggaggagggcgagttctccgaggcgcgcgaggatctcgctgcgctggagaaggactacgaggaggttggcgctgagtccgccgacgacatgggtgaggaggacgtcgaggagtacatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacgatgagcatcgtgaactcgtacgtgaacgacgtgatggagcgcatctgcatggaggccgcgtcgatcgttcgcgcgaacaagaagcgcacgttgggtgcgcgcgaggtgcagacggcggtgcgcattgtgctgccggcggagctcgcgaagcacgccatggctgagggcacgaaggccgtgtcgagcgcgtcggct

SEQ ID NO: 47 - protein, 1076 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMVNVCVVGAAGGIGQSLSLLLVRQLPYGSTLSLFDVVGAAGVAADLSHVDNAGVQVKFAAGKIGQKRDPALAELAKGVDVFVMVAGVPRKPGMTRDDLFKINAGIILDLVLTCASSSPKAVFCIVTNPVNSTVVIAAEALKSLGVYDRNRLLGVSLLDGLRATCFINEARKPLVVTQVPVVGGHSDATIVPLFHQLLGPLPEQATLDKIVKRVQVAGTEVVKAKAGRGSATLSMAEAGARFTLKVVEGLTGTGKPLVYAYVDTDGQHETPFLAIPVVLGVNGIEKRLPIGPLHSTEETLLKAALPVIKKNIVKGSEFARSHLMRDAHTRTPTEKKTRSSSLSFFEQTPLNRLLTPLSSFSAMREAICIHIGQAGCQVGNACWELFCLEHGIQPDGSMPSDKCIGVEDDAFNTFFSETGAGKHVPRCIFLDLEPTVVDEVRTGTYRQLFNPEQLVSGKEDAANNYARGHYTIGKEIVDLALDRIRKLADNCTGLQGFMVFHAVGGGTGSGLGALLLERLSVDYGKKSKLGYTVYPSPQVSTAVVEPYNCVLSTHSLLEHTDVATMLDNEAIYDLTRRSLDIERPSYTNVNRLIGQVVSSLTASLRFDGALNVDLTEFQTNLVPYPRIHFVLTSYAPVVSAEKAYHEQLSVADITNSVFEPAGMLTKCDPRHGKYMSCCLMYRGDVVPKDVNAAIATIKTKRTIQFVDWCPTGFKCGINYQPPTVVPGGDLAKVQRAVCMIANSTAIAEVFARIDHKFDLMYSKRAFVHWYVGEGMEEGEFSEAREDLAALEKDYEEVGAESADDMGEEDVEEYMASSRSAPRKASHAHKSHRKPKRSWNVYVGRSLKAINAQMSMSHRTMSIVNSYVNDVMERICMEAASIVRANKKRTLGAREVQTAVRIVLPAELAKHAMAEGTKAVSSASA

SEQ ID NO: 48 - DNA, 3132 bp, Artificial Sequence
  Synthetic Construct
atgaaggacaaggcgacgggcaagacgcagaacatcacgatcacggcgaacggcgggctgtcgaaggagcagatcgagcagatgatccgcgactcggagcagcacgcggaggccgaccgcgtgaagcgcgagcttgtggaggtgcgcaacaacgcggagacgcagctgacaacggcggagaggcagctcggcgagtggaagtacgtgagcgatgcggagaaggagaacgtgaagacgctggtggcggagctgcgcaaggcgatggagaacccgaacgtcgcgaaggatgaccttgcggctgcgacggacaagctgcagaaggctgtgatggagtgcggccgcacagagtaccagcaggctgccgcggccaactccggcagcaccagcaactccggtgagcagcagcagcagcagggccaaggtgagcagcagcagcagcagaacagcgaagagaagaagatgcgcgatgcacacacgcgcacgcccaccgaaaaaaaaacgcgcagctcttcgctctcgttcttcgaacaaacacctttaaaccgccttctaacccctctttcttctttttcagccatgcgtgaggctatctgcatccacatcggccaggccggctgccaggtcggtaacgcgtgctgggagctgttctgccttgagcacggcatccagcctgatggctccatgccctctgacaagtgcatcggtgttgaggatgacgcgttcaacacgttcttctcggagactggtgctggcaagcacgttcctcgctgcatcttcctggacctcgagcctacggtcgtggatgaggtgcgcaccggcacgtaccgccagctgttcaaccccgagcagctggtgtccggcaaggaggatgcggcgaacaactacgctcgtggccactacaccatcggcaaggagatcgtcgaccttgcgctggaccgcattcgcaagctggcggacaactgcacgggtctccagggctttatggtgttccacgctgtgggtggcggcaccggctctggcctcggtgcgctgctgctggagcgcctgtctgtggactacggcaagaagtccaagcttggctacaccgtgtacccgagcccgcaggtgtcgactgccgtcgtggagccgtacaactgcgtgctgtcgacgcactcgctgctcgagcacaccgatgttgcgacgatgctcgacaatgaggccatctacgacctcactcgtcgttctctcgacattgagcgcccgtcgtacacgaacgtgaaccgcctgatcggccaggtggtgtcgtctctgacggcgtcgctgcgcttcgatggtgcgctgaacgtggacctgacggagttccagacgaaccttgtgccgtacccgcgcatccacttcgtgctgacgagctatgctccggtggtgtctgccgagaaggcgtaccacgagcagctgtccgtcgcggacatcacgaactcggtgtttgagcctgctggcatgctgacgaagtgcgatcctcgccacggcaagtacatgtcgtgctgcctcatgtaccgcggtgatgtcgtgccgaaggatgtcaacgccgcgattgcgacgatcaagacgaagcggacaattcagttcgtggactggtgtccgaccggcttcaagtgcggcatcaactaccagccgccgaccgttgtgcccggcggtgacctcgcgaaggtgcagcgcgccgtgtgcatgattgccaactcgaccgcgatcgctgaggtgtttgcccgcatcgaccacaagttcgacctgatgtacagcaagcgcgcgttcgtgcactggtacgtgggtgagggcatggaggagggcgagttctccgaggcgcgcgaggatctcgctgcgctggagaaggactacgaggaggttggcgctgagtccgccgacgacatgggtgaggaggacgtcgaggagtactcggcggtcggcaacatcgagtcgcagtgggcccgtgccggccacggcttggtgagcctgtcggagcagcagctggtgagctgcgatgacaaagacaatggctgcaacggcgggctgatgctgcaggcgttcgagtggctgctgcgacacatgtacgggatcgtgttcacggagaagagctacccctacacgtccggcaacggtgatgtggccgagtgcttgaacagcagtaaactcgttcccggcgcgcaaatcgacggctacgtgatgatcccgagcaacgaaacggttatggctgcgtggcttgcggagaatggccccatcgcgattgcggtcgacgccagctccttcatgtcttaccagagcggcgtgctgaccagctgcgctggcgatgcactgaaccacggcgtgctgctcgtcgggtacaacaagaccggtggggttccgtactgggtgatcaagaactcgtggggtgaggactggggcgagaagggctacgtgcgcgtggtcatggggctgaacgcgtgcctgctcagtgaataccccgtgtccgcgcatgtgccgcggagtctcacccctggcccgggcacggagagcgaggagcgcgcccctaaacgggtgacggtggagcagatgatgtgcaccgatatgtactgcagggaggggtgcaagaagagtcttctcaccgcgaacgtgtgctacaagaacgggggaggcggctcctctatgacgaagtgcggtccgcagaaggtgctgatgtgctcgtactcgaaccctcattgctttggtcctgggctgtgcctcgagactcctgatggcaagtgcgcgccgtacttcttgggctcgatcatgaacacctgccagtacacgatggcctcttctcgctctgctccccgcaaggcttcccacgcgcacaagtcgcaccgcaagccgaagcgctcgtggaacgtgtacgtgggccgctcgctgaaggcgatcaacgcccagatgtcgatgtcgcaccgcacgatgagcatcgtgaactcgtacgtgaacgacgtgatggagcgcatctgcatggaggccgcgtcgatcgttcgcgcgaacaagaagcgcacgttgggtgcgcgcgaggtgcagacggcggtgcgcattgtgctgccggcggagctcgcgaagcacgccatggctgagggcacgaaggccgtgtcgagcgcgtcggct

SEQ ID NO: 49 - protein, 1044 aa, Artificial Sequence
  Synthetic Construct
MKDKATGKTQNITITANGGLSKEQIEQMIRDSEQHAEADRVKRELVEVRNNAETQLTTAERQLGEWKYVSDAEKENVKTLVAELRKAMENPNVAKDDLAAATDKLQKAVMECGRTEYQQAAAANSGSTSNSGEQQQQQGQGEQQQQQNSEEKKMRDAHTRTPTEKKTRSSSLSFFEQTPLNRLLTPLSSFSAMREAICIHIGQAGCQVGNACWELFCLEHGIQPDGSMPSDKCIGVEDDAFNTFFSETGAGKHVPRCIFLDLEPTVVDEVRTGTYRQLFNPEQLVSGKEDAANNYARGHYTIGKEIVDLALDRIRKLADNCTGLQGFMVFHAVGGGTGSGLGALLLERLSVDYGKKSKLGYTVYPSPQVSTAVVEPYNCVLSTHSLLEHTDVATMLDNEAIYDLTRRSLDIERPSYTNVNRLIGQVVSSLTASLRFDGALNVDLTEFQTNLVPYPRIHFVLTSYAPVVSAEKAYHEQLSVADITNSVFEPAGMLTKCDPRHGKYMSCCLMYRGDVVPKDVNAAIATIKTKRTIQFVDWCPTGFKCGINYQPPTVVPGGDLAKVQRAVCMIANSTAIAEVFARIDHKFDLMYSKRAFVHWYVGEGMEEGEFSEAREDLAALEKDYEEVGAESADDMGEEDVEEYSAVGNIESQWARAGHGLVSLSEQQLVSCDDKDNGCNGGLMLQAFEWLLRHMYGIVFTEKSYPYTSGNGDVAECLNSSKLVPGAQIDGYVMIPSNETVMAAWLAENGPIAIAVDASSFMSYQSGVLTSCAGDALNHGVLLVGYNKTGGVPYWVIKNSWGEDWGEKGYVRVVMGLNACLLSEYPVSAHVPRSLTPGPGTESEERAPKRVTVEQMMCTDMYCREGCKKSLLTANVCYKNGGGGSSMTKCGPQKVLMCSYSNPHCFGPGLCLETPDGKCAPYFLGSIMNTCQYTMASSRSAPRKASHAHKSHRKPKRSWNVYVGRSLKAINAQMSMSHRTMSIVNSYVNDVMERICMEAASIVRANKKRTLGAREVQTAVRIVLPAELAKHAMAEGTKAVSSASA

SEQ ID NO: 50 - DNA, 21 bp, Artificial Sequence
  Synthetic Construct
atgcatcaccatcaccatcac